(19) United States
(12) Patent Application Publication
(10) Pub. No.: US 2024/0225894 A1
(43) Pub. Date: Jul. 11, 2024

(54) OCULAR DELIVERY SYSTEMS AND METHODS

(71) Applicant: Sight Sciences, Inc., Menlo Park, CA (US)

(72) Inventors: David Samuel NEEDLEMAN, San Carlos, CA (US); Frederick James SERRA-HSU, Fremont, CA (US); Reay Hugh BROWN, Atlanta, GA (US)

(21) Appl. No.: 18/454,585

(22) Filed: Aug. 23, 2023

Related U.S. Application Data
(60) Provisional application No. 63/400,267, filed on Aug. 23, 2022.

(51) Int. Cl. A61F 9/00; A61F 9/007

(52) U.S. Cl. CPC A61F 9/0017; A61F 9/00781; A61F 2250/0068

(57) ABSTRACT

Disclosed herein are devices for delivering fluid to an eye. The device may include a handle comprising a housing at least partially containing a fluid reservoir therein, a cannula coupled to a distal end of the housing, and an elongate member slidably positioned within the cannula. The device may include a connector releasably coupled to the fluid reservoir in the handle, where the connector is configured to receive an external fluid device to transfer fluid into the fluid reservoir, and to be released from the handle with the external fluid device coupled to the connector. The cannula may include a curved proximal portion having a first radius of curvature, a curved distal portion having a second radius of curvature, and a distal tip, with the first radius of curvature greater than the second radius of curvature, and where the first and second radii of curvature are in opposing directions.

### INCORPORATION BY REFERENCE

This application claims priority to U.S. Provisional Application Ser. No. 63/400,267 filed on Aug. 23, 2022, which is hereby incorporated by reference in its entirety.

### TECHNICAL FIELD

This invention relates generally to fluid delivery systems for treating conditions of the eye, and associated methods for treating such conditions of the eye.

### BACKGROUND

Glaucoma is a potentially blinding disease that affects over 60 million people worldwide, or about 1-2% of the population. Typically, glaucoma is characterized by elevated intraocular pressure. Increased pressure in the eye can cause irreversible damage to the optic nerve which can lead to loss of vision and even progress to blindness if left untreated. Consistent reduction of intraocular pressure can slow down or stop progressive loss of vision associated with glaucoma.

Increased intraocular pressure is generally caused by sub-optimal efflux or drainage of fluid (aqueous humor) from the eye. Aqueous humor or fluid is a clear, colorless fluid that is continuously replenished in the eye. Aqueous humor is produced by the ciliary body, and then ultimately exits the eye primarily through the trabecular meshwork. The trabecular meshwork extends circumferentially around the eye at the anterior chamber angle, or drainage angle, which is formed at the intersection between the peripheral iris or iris root, the anterior sclera or scleral spur and the peripheral cornea. The trabecular meshwork feeds outwardly into Schlemm's canal, a narrow circumferential passageway generally surrounding the exterior border of the trabecular meshwork. Positioned around and radially extending from Schlemm's canal are aqueous veins or collector channels that receive drained fluid. The net drainage or efflux of aqueous humor can be reduced as a result of decreased facility of outflow, decreased outflow through the trabecular meshwork and canal of Schlemm drainage apparatus, increased episcleral venous pressure, or possibly, increased production of aqueous humor. Flow out of the eye can also be restricted by blockages or constriction in the trabecular meshwork and/or Schlemm's canal and its collector channels.

Glaucoma, pre-glaucoma, and ocular hypertension currently can be treated by reducing intraocular pressure using one or more modalities, including medication, incisional surgery, laser surgery, cryosurgery, and other forms of surgery. In general, medications or medical therapy are the first lines of therapy. If medical therapy is not sufficiently effective, more invasive surgical treatments may be used. For example, a standard incisional surgical procedure to reduce intraocular pressure is trabeculectomy, or filtration surgery. This procedure involves creating a new drainage site for aqueous humor. Instead of naturally draining through the trabecular meshwork, a new drainage pathway is created by removing a portion of sclera and trabecular meshwork at the drainage angle. This creates an opening or passage between the anterior chamber and the subconjunctival space that is drained by conjunctival blood vessels and lymphatics. The new opening may be covered with sclera and/or conjunctiva to create a new reservoir called a bleb into which aqueous humor can drain. However, traditional trabeculectomy procedures carry both short- and long-term risks. These risks include blockage of the surgically created opening through scarring or other mechanisms, hypotony or abnormally low intraocular pressure, expulsive hemorrhage, hyphema, intraocular infection or endophthalmitis, shallow anterior chamber angle, macular hypotony, choroidal exudation, suprachoroidal hemorrhage, and others.

One alternative is to implant a device in Schlemm's canal that maintains the patency of the canal or aids flow of aqueous humor from the anterior chamber into the canal. Various stents, shunts, catheters, and procedures have been devised for this purpose and employ an ab-externo (from the outside of the eye) approach to deliver the implant or catheter into Schlemm's canal. This method of placement is invasive and typically prolonged, requiring the creation of tissue flaps and deep dissections to access the canal. Additionally, it is very difficult for many surgeons to find and access Schlemm's canal from this external incisional approach because Schlemm's canal has a small diameter, e.g., approximately 50 to 250 microns in cross-sectional diameter, and it may be even smaller when collapsed. One related non-implant procedure, ab-externo canaloplasty, involves making a deep scleral incision and flap, finding and unroofing Schlemm's canal, circumnavigating all 360 degrees of the canal with a catheter from the outside of the eye, and either employing viscoelastic, a circumferential tensioning suture, or both to help maintain patency of the canal. The procedure is quite challenging and can take anywhere from forty-five minutes to two hours. The long-term safety and efficacy of canaloplasty is very promising, but the procedure remains surgically challenging and invasive.

Another alternative is viscocanalostomy, which involves the injection of a viscoelastic solution into Schlemm's canal to dilate the canal and associated collector channels. Dilation of the canal and collector channels in this manner generally facilitates drainage of aqueous humor from the anterior chamber through the trabecular meshwork and Schlemm's canal, and out through the natural trabeculocanalicular outflow pathway. Viscocanalostomy is similar to canaloplasty (both are invasive and ab-externo), except that viscocanalostomy does not involve a suture and does not restore all 360 degrees of outflow facility. Some advantages of viscocanalostomy are that sudden drops in intraocular pressure, hyphema, hypotony, and flat anterior chambers may be avoided. The risk of cataract formation and infection may also be minimized because of reduced intraocular manipulation and the absence of full eye wall penetration, anterior chamber opening and shallowing, and iridectomy. A further advantage of viscocanalostomy is that the procedure restores the physiologic outflow pathway, thus avoiding the need for external filtration, and its associated short and long term risks, in the majority of eyes. This makes the success of the procedure partly independent of conjunctival or episcleral scarring, which is a leading cause of failure in traditional trabeculectomy procedures. Moreover, the absence of an elevated filtering bleb avoids related ocular discomfort and potentially devastating ocular infections, and the procedure can be carried out in any quadrant of the outflow pathway.

However, ab externo viscocanalostomy and canaloplasty techniques are still very invasive because access to Schlemm's canal must be created by making a deep incision into the sclera, creating a scleral flap, and un-roofing Schlemm's canal. “Ab-externo” generally means “from the outside” and it is inherently more invasive given the location of Schlemm's canal and the amount of tissue disruption required to access it from the outside. On the other hand, “ab-interno” means “from the inside” and is a less invasive approach because of the reduced amount of tissue disruption required to access it from the inside. Consequently, an ab-interno approach to Schlemm's canal offers the surgeon easier access to the canal, but also reduces risk to the patient's eye and reduces patient morbidity. All of these lead to improved patient recovery and rehabilitation. The ab-externo viscocanalostomy and canaloplasty procedures also remain challenging to surgeons, because as previously stated, it is difficult to find and access Schlemm's canal from the outside using a deep incisional approach due to the small diameter of Schlemm's canal. A further drawback still is that at most, viscocanalostomy typically dilates up to 60 degrees of Schlemm's canal, which is a 360-degree ring-shaped outflow vessel-like structure. The more of the canal that can be dilated, the more total aqueous outflow can be restored.

Accordingly, it would be beneficial to have systems that easily and atraumatically provide access to Schlemm's canal using an ab-interno approach for the delivery of tools and fluid compositions. It would also be useful to have systems that deliver tools and compositions into Schlemm's canal expeditiously to decrease procedure time and the risk of infection without compromising safety and precision of the delivery procedure. It would also be useful to have systems that deliver tools and fluid compositions into Schlemm's canal using an ab-interno approach so that cataract surgery and glaucoma surgery can both be accomplished during the same surgical sitting using the very same corneal or scleral incision. Such incisions are smaller and allow for less invasive surgery and more rapid patient recovery. This approach allows for accessing Schlemm's canal through the trabecular meshwork from the inside of the eye, and thus it is called “ab-interno.” Methods of delivering tools and compositions that effectively disrupt the juxtacanalicular meshwork and adjacent wall of Schlemm's canal, also known as the inner wall of Schlemm's canal, maintain the patency of Schlemm's canal, increase outflow, decrease resistance to outflow, or effectively dilate the canal and/or its collector channels using the systems in a minimally invasive, ab-interno manner would also be desirable.

### SUMMARY

Disclosed herein is a device for delivering fluid to an eye. The device may include a handle comprising a housing containing a fluid reservoir, a cannula coupled to a distal end of the handle, an elongate member slidably positioned within the cannula. The device may further include a connector releasably coupled to the fluid reservoir in the handle, where the connector may be configured to receive an external fluid device to transfer fluid into the fluid reservoir, and to be released from the handle with the external fluid device coupled to the connector.

In some variations, the handle may include a proximal portion comprising a proximal cavity having a proximal opening, the proximal cavity having a coupling hub within the proximal cavity, the coupling hub including a coupling portion. In some variations, the connector may engage the coupling portion of the coupling hub, and the coupling portion may include threads configured to engage a distal luminal wall of the connector.

In some variations, the handle may comprise a sealing member distal the coupling hub, the sealing member configured to seal the fluid reservoir.

In some variations, the connector may comprise a connector body including one or more extensions configured to engage one or more abutments within the proximal cavity of the handle when the connector is coupled to the coupling hub. The coupling hub may include a plug having a plug lumen in fluid communication with the fluid reservoir.

In some variations, the housing may comprise a distal portion of the handle, the distal portion may include a grip portion proximal a distal end of the handle.

In some variations, the grip portion may include a textured surface having raised elements, indented elements, or a combination. The textured surface may include raised elements or indented elements having the same shape. The textured surface may include raised elements or indented elements having different shapes. The textured surface may include raised elements or indented elements having the same cross-sectional area or the same diameter or different cross-sectional areas or different diameters.

In some variations, the grip portion may include a top surface and a bottom surface, where the top surface or the bottoms surface may include one or more actuators.

The grip portion may be symmetrical across a YZ plane, across a XZ plane, or symmetrical across each of the YZ plane and the XZ plane.

In some variations, the grip portion may include a maximum height at the one or more actuators and a minimum height at the distal end of the handle.

In some variations, the grip portion may comprise two or more cross-sectional shapes along a longitudinal axis of the handle.

In some variations, a center section of the grip portion may comprise an oval cross-sectional shape with a major and minor axis, the center section of the grip portion including the top surface, the bottom surface, and the one or more actuators. The grip portion may comprise a circular cross-sectional shape with a first diameter proximal the center section and a circular cross-sectional shape with a second diameter distal the center section, wherein the first diameter is greater than the second diameter.

Also disclosed herein is a method of delivering fluid to treating conditions of the eye. The method may include coupling an external fluid device to a connector of a handle of a delivery device, where the delivery device may include the handle comprising housing, a cannula coupled to the handle, a fluid reservoir, and an elongate member, in fluid communication with the fluid reservoir, slidably positioned within the cannula. The method includes delivering fluid from an external fluid device through a connector releasably coupled to the fluid reservoir contained within the housing of the delivery device and removing the connector with the external fluid device coupled to the connector from the delivery device. The method may further include advancing the cannula of the delivery device to the eye, advancing the elongate member around Schlemm's canal, and delivering fluid to the eye through the elongate member.

In some variations, removing the connector and the external fluid device coupled to the connector from the delivery device may include rotating the connector to remove the connector and the external fluid device from the delivery device. Removing the connector and the external fluid device coupled to the connector from the delivery device may include removing the connector from a proximal cavity within the handle. Removing the connector and the external fluid device coupled to the connector from the delivery device includes rotating the connector to disengage the connector from a coupling hub within the proximal cavity of the handle. Removing the connector and the external fluid device coupled to the connector from the delivery device includes rotating the connector using one or more tabs extending from a connector body in a second direction opposite a first direction.

Also disclosed herein is a device for delivering fluid to an eye. The device may include a handle containing a fluid reservoir, a cannula coupled to a distal end of the handle, the cannula having a curved proximal portion, a curved distal portion, and a distal tip, where the curved proximal portion has a first radius of curvature and the curved distal portion has a second radius of curvature, where the first radius of curvature is greater than the second radius of curvature, and where the first and second radii of curvature are in opposing directions. The device may further include an elongate member slidably positioned in the cannula and configured to deliver fluid to Schlemm's canal.

The device further includes the distal tip further comprising a distal edge having a straight portion, a proximal edge having a curved portion, and a lumen opening of the cannula. In some variations, the distal tip may be located on an inner radius of the cannula or on an outer radius of the cannula.

In some variations, the straight portion of the distal edge and the outer radius may form an angle between about 14 degrees to about 20 degrees. The distal edge or the proximal edge may be chamfered, or the distal edge and the proximal edge may be chamfered.

The distal edge may include an outer distal edge and an inner distal edge defining a tongue. The proximal edge may further comprise an inner proximal edge and an outer proximal edge defining a base. The distal edge may be rounded or straight.

In some variations, a diameter of the distal tip may taper from the proximal edge to the distal edge.

In some variations, the straight portion of the distal tip may comprise one or more straight segments. The straight portion of the distal tip may comprise a plurality of straight segments where each segment comprises a different slope.

In some variations, the curved portion of the distal tip may comprise one or more curved segments. The one or more curved segments may include a plurality of curved segments where each segment comprises a different radius of curvature. In some variations, the distal tip may have a length along a longitudinal axis from the distal edge to the proximal edge.

In some variations, the length of the straight portion may comprise a greater percentage of the length of the distal tip than a length of the curved portion. In some variations, a length of the curved portion comprises a greater percentage of the length of the distal tip than a length of the straight portion.

In some variations, the cannula may further comprise an outer wall and an inner wall defining a wall thickness. In some variations, the wall thickness tapers along the length of the distal tip. The wall thickness may taper along the length of the straight portion. In some variations, the wall thickness tapers along a length of a tongue of the straight portion. The wall thickness may be constant along the length of the distal tip. The wall thickness may be constant along the length of the straight portion. The wall thickness may be constant along a length of a tongue of the straight portion.

In some variations, the distal tip of the cannula may be aligned with a central longitudinal axis of the handle or may be bisected by the central longitudinal axis of the handle. In some variations, the curved proximal portion and the curved distal portion may be offset from the central longitudinal axis of the handle.

Also disclosed herein is a device for delivering fluid to the eye. The device may include a handle containing a fluid reservoir at least partially within the handle, the handle may comprise a housing having a proximal portion, and a distal portion comprising a grip portion. The grip portion may comprise a first curved side, a second curved side opposite the first curved side, and a tapered region distal to the first and second curved sides, where the tapered region is configured to receive fingers of the user. The device may further comprise a cannula coupled to a distal end of the handle, and an elongate member slidably positioned in the cannula and configured to deliver fluid to Schlemm's canal.

In some variations, the first and second curved sides are convex. The first and second curved sides may be symmetric across an XZ plane parallel to a longitudinal midpoint.

In some variations, the grip portion may comprise an actuator configured to move the elongate member and/or deliver fluid. The grip portion may further comprise an actuator border around the actuator.

In some variations, the grip portion may further comprise a planar surface proximal the actuator. The planar surface may include a first planar surface and the grip portion may further comprise a second planar surface, where the first planar surface is on a top of the handle and the second planar surface is on a bottom of the handle. In some variations, the grip portion may comprise a neck proximal of the actuator.

In some variations, the proximal portion may have a first height at a distal end, the neck may have a second height, and the distal portion may have a maximum height aligned with at least a portion of the actuator, and the first height and the maximum height may be greater than the second height. In some variations, the grip portion may comprise a non-slip material on the tapered portion. The grip portion may comprise an actuator configured to move the elongate member and/or deliver fluid, and the non-slip material extends beyond the actuator and terminates adjacent to the distal end of the handle. The non-slip material may surround the tapered portion.

### BRIEF DESCRIPTION OF THE DRAWINGS

FIG. 1 shows a stylized, cross-sectional view of the eye and some of the structures involved in the flow of aqueous humor out of the eye.

FIG. 2 depicts a perspective view of an exemplary delivery device.

FIG. 3 illustrates an exploded view of a variation of an exemplary delivery device.

FIG. 4A depicts a perspective view of the connector of the delivery system and FIG. 4B depicts a front view of the connector of FIG. 4A.

FIG. 5A depicts a perspective view of an exemplary delivery device having a handle including a housing comprising a grip portion. FIG. 5B depicts a front view of the delivery device of FIG. 5A. FIG. 5C depicts a side view of the exemplary delivery device of FIG. 5A.

FIG. 6A depicts a perspective view of a proximal end of a handle of the delivery device including a connector of the delivery system. FIG. 6B depicts the proximal end including the proximal opening of the handle of FIG. 6A. FIG. 6C depicts a rear view of the proximal end of the handle of FIG. 6A.

FIG. 7 depicts a side view of an exemplary cannula of the delivery device.

FIGS. 8A-8E depict side views of exemplary variations of the cannula.

FIG. 9A illustrates a variation of a distal tip of the cannula. FIG. 9B illustrates a side view of the distal tip of FIG. 9A and FIG. 9C depicts a side view of a variation of the distal tip of FIG. 9A.

FIG. 10 depicts a side view of the distal tip of the cannula.

FIGS. 11A-11L depict further variations of a distal tip of the cannula.

FIGS. 12A-12C depict an exemplary delivery device showing a method of delivering fluid from an external fluid device to the delivery device, in accordance with some variations.

FIG. 13 is a flow chart of an exemplary method for delivering a fluid to Schlemm's canal using a variation of the delivery system described herein.

FIGS. 14A-14E depict side views of a distal end of the elongate member, in accordance with some variations.

### DETAILED DESCRIPTION

Described here are systems and methods for accessing Schlemm's canal and for delivering a fluid composition therein and/or for tearing the trabecular meshwork to reduce intraocular pressure and thereby treat conditions of the eye. The fluids and certain components of the system, e.g., the slidable elongate member (e.g., conduit), may be used to provide a force for disrupting trabeculocanalicular tissues, which include the trabecular meshwork, juxtacanalicular tissue, Schlemm's canal, and the collector channels. As used herein, the term “disrupting” refers to the delivery of a volume of fluid or a system component that alters the tissue in a manner that improves flow through the trabeculocanalicular outflow pathway. Examples of tissue disruption include, but are not limited to, dilation of Schlemm's canal, dilation of collector channels, increasing the porosity of the trabecular meshwork, stretching the trabecular meshwork, forming microtears or perforations in juxtacanalicular tissue, removing septae from Schlemm's canal, cutting, tearing, or removal of trabeculocanalicular tissues, or a combination thereof.

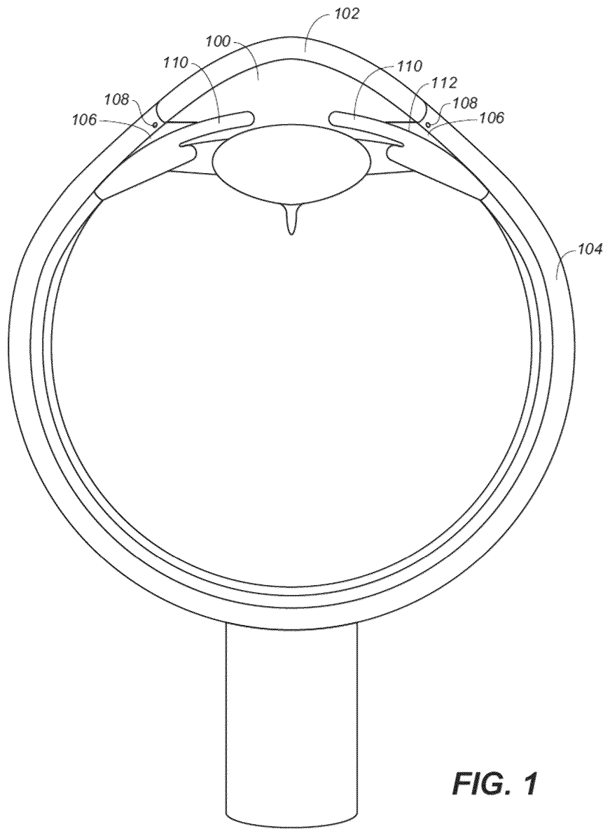

To better understand the systems and methods described here, it may be useful to explain some of the basic eye anatomy. FIG. 1 is a stylized depiction of a normal human eye. The anterior chamber (100) is shown as bounded on its anterior surface by the cornea (102). The cornea (102) is connected on its periphery to the sclera (104), which is a tough fibrous tissue forming the protective white shell of the eye. Trabecular meshwork (106) is located on the outer periphery of the anterior chamber (100). The trabecular meshwork (106) extends 360 degrees circumferentially around the anterior chamber (100). Located on the outer peripheral surface of the trabecular meshwork (106) is Schlemm's canal (108). Schlemm's canal (108) extends 360 degrees circumferentially around the meshwork (106). At the apex formed between the iris (110), meshwork (106), and sclera (104), is the anterior chamber angle (112).

The delivery systems are generally configured for single-handed manipulation and for control by a single operator and include one or more features useful for easily accessing Schlemm's canal with minimal trauma. Once access to the canal has been obtained, the delivery system may deliver a fluid composition and/or tear the trabecular meshwork. For example, the lumen of the elongate member (e.g., conduit) may be configured to deliver a fluid composition to the canal, and the body of the elongate member may be configured to cut or tear through the trabecular meshwork if the system is removed from the eye while the elongate member is extended from the cannula and within Schlemm's canal.

It should be appreciated that in some instances the delivery systems described herein may be used only to deliver a fluid composition to Schlemm's canal (and not to separately tear the trabecular meshwork using, for example, the body of the elongate member) or may be used only to tear the trabecular meshwork (using, for example, the body of the elongate member) without delivering a fluid composition. Moreover, as noted above, in some instances, the delivery systems described herein may be used to both deliver a fluid composition to Schlemm's canal and to tear the trabecular meshwork.

In some variations the methods described herein may comprise implanting a device completely or partially into Schlemm's canal in conjunction with delivering a fluid composition into the canal and/or tearing the trabecular meshwork. When a device is implanted into the canal, it will generally be configured to maintain the patency of Schlemm's canal without substantially interfering with transmural fluid flow across the canal. This may restore, enable, or enhance normal physiologic efflux of aqueous humor through the trabeculocanalicular tissues. Ocular implants such as those disclosed in U.S. Pat. No. 7,909,789, and such as those disclosed in U.S. Pat. No. 8,529,622, each of which is hereby incorporated by reference in its entirety, may be delivered. In some variations, the implants in U.S. Pat. Nos. 7,909,789 and 8,529,622 include a support having a least one fenestration that completely traverses a central core of Schlemm's canal without substantially interfering with transmural fluid flow or longitudinal fluid flow across or along the canal. The ocular device may also disrupt the juxtacanalicular trabecular meshwork or adjacent inner wall of Schlemm's canal. The ocular devices may also be coated with a drug useful for treating ocular hypertension, glaucoma, or pre-glaucoma, infection, or scarring, neovascularization, fibrosis, or inflammation postoperatively. The ocular device may also be formed to be solid, semi-solid, or bioabsorbable.

### Delivery Devices and Systems

The systems described herein may generally include single-handed, single-operator controlled devices and one or more external fluid delivery devices configured to deliver fluid to the delivery devices and releasably couple to the delivery devices. The delivery devices generally comprise a handle having a grip portion and a housing that has an interior and a distal end, and a connector configured to be detachably couple to both the housing of the handle and the one or more external fluid devices. The delivery devices may include a cannula coupled to and extending from the distal end of the housing. The cannula may include a proximal end, a straight portion, and a distal portion, where the curved portion has a proximal end and a distal end, and one or more radii of curvature. In other variations, the entire cannula may be straight, and may therefore not comprise a distal curved portion. The cannula may also include a lumen extending from the proximal end through the distal tip, and the distal tip may include an opening to the lumen.

The devices may also generally include a drive assembly partially contained within the housing. In some variations, the drive assembly may comprise one or more gears that translate rotational movement into linear movement. The delivery devices may also include a slidable elongate member coaxially disposed within the cannula lumen. In some variations, the slidable elongate member may comprise a lumen therethrough. The delivery devices may also include a fluid assembly housed at least partially in the handle. Fluid compositions such as saline, viscoelastic fluids, including viscoelastic solutions, air, and gas may be delivered using the delivery device of the system. Suitable markings, colorings, or indicators may be included on any portion of the delivery device of the system to help identify the location or position of the distal end of the cannula and/or the slidable elongate member.

In some instances, the systems described herein may be used to perform ab-interno trabeculotomy, ab-interno transluminal trabeculotomy, clear corneal trabeculotomy, clear corneal transluminal trabeculotomy, ab-interno canaloplasty, and/or clear corneal canaloplasty, and/or may be used to deliver a fluid composition into the anterior or posterior segment of the eye.

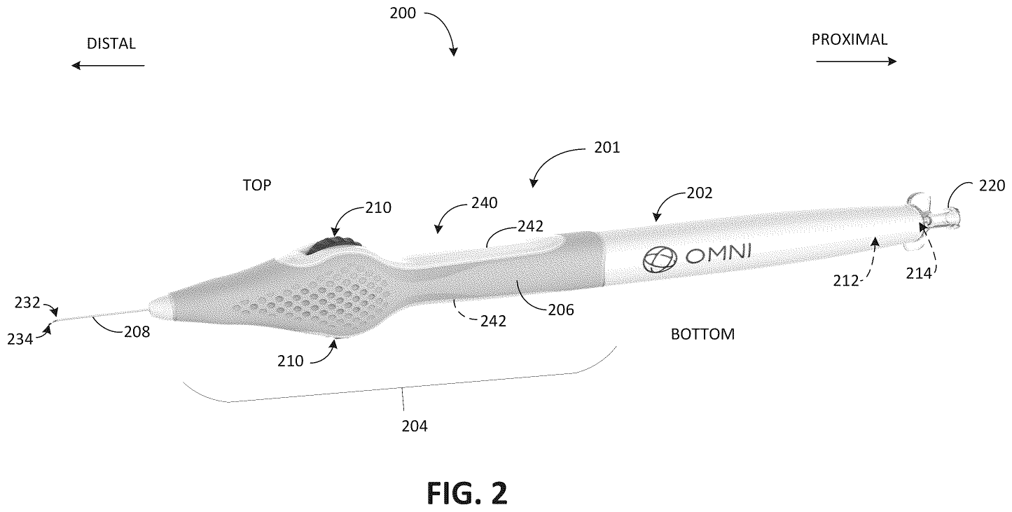

Part of an exemplary delivery system is depicted in FIG. 2. As shown there, the delivery system (200) may include a delivery device (201) having a universal handle (202) comprising a housing (206) including a grip portion (204). The housing (206) has a proximal end and a distal end. A cannula (208) comprising a distal tip (232) may be coupled to and may extend from the distal end of the housing. The delivery device (201) may further include an elongate member (234) slidably positioned within the cannula (208). The distal tip (232) of the cannula (208) may be configured to provide the user enhanced functionality in seating the cannula (208) in Schlemm's canal, as will be described in more detail herein. The delivery device (201) may further comprise a drive assembly substantially contained within the housing (206). The drive assembly may be configured to actuate movement of the elongate member (234) and/or deliver a fluid composition to the eye. For example, as shown in FIG. 2, the drive assembly may comprise one or more actuators (210) (e.g., one, two, three, four, or more) such as a rotatable element (e.g., wheel), slide, button, or the like, actuation of which (e.g., rotation, translation, depression) may advance and/or retract the slidably elongate member and/or may deliver the fluid composition. In some variations, the one or more actuators (210) may extend out of the housing (206) to facilitate user access. For example, the one or more actuators (210) may extend out of the housing of the handle, such as, on opposing sides of the handle as depicted in the variation shown in FIG. 2. As will also be described in more detail herein, the handle (202) is configured, (e.g., the size, shape (including curvatures), placement of portions/components (e.g., actuators, grip portion) of the handle, etc.) to ergonomically fit within the hand of a user and provide easy and comfortable access to the actuators and rotation of the handle itself along a longitudinal axis of the handle thus allowing the user to easily control the movement of the elongate member and/or fluid delivery.

The delivery device (201) of the delivery system (200) may further comprise a fluid assembly comprising a reservoir (212) having a proximal opening (214), and a connector (220) configured to releasably couple to the handle (202) of the delivery device (201). When the connector (220) is coupled to the handle (202), the proximal opening (214) of the reservoir (212) may be in fluid communication with the connector (220), such that the fluid composition may be delivered to the reservoir (212) through the connector (220). The connector (220) may be configured to detachably couple to (e.g., releasably receive at least partially therein) an external fluid device. In this manner, the connector (220) may be configured to easily allow a volume of the fluid composition to be transferred from the external fluid device to the reservoir (212). After transfer of the volume of the fluid composition, the connector (220) may be decoupled from the delivery device (201) to assist in removing, releasing, or otherwise decoupling the external fluid device from the housing (206) of the handle (202) of the delivery device (201). The delivery system (200) including the delivery device (201) will be described in more detail herein.

The delivery systems or components thereof described herein may in some variations be fully disposable. In other variations, a portion of the delivery system may be reusable (e.g., non-patient contact materials, such as the handle (202)), while a portion of the delivery system may be disposable (e.g., patient-contact materials, such as the cannula (208) and the elongate member (234)). In yet other variations, the delivery systems described herein may be fully reusable.

### External Fluid Device and Fluid Compositions

In some variations, an external fluid device may be coupled to the connector to deliver a fluid composition to the delivery device. The external fluid device may include a syringe, a vial, or another container used to store fluid. The external fluid device may be provided as part of the delivery system in a kit, or the external fluid device may be separately provided by the user. In some variations, the external fluid device may be pre-loaded with a fluid composition and may be packaged, e.g., in a kit, with a fluid composition disposed therein. In other variations, the external fluid device may not be pre-loaded, and may instead be packaged empty and loaded with a fluid composition by the user prior to a procedure. In these variations, the kits described herein may also include a separate fluid container (e.g., vial, syringe) containing fluid for use with the external fluid devices and/or fluid delivery devices described herein. The external fluid device may include volume markings. In some variations, the external fluid device may be partially or entirely transparent or otherwise see through, to allow the user to easily distinguish the volume of the fluid composition within the external fluid device.

The fluid composition may comprise fluid compositions including but not limited to saline and viscoelastic fluids. The viscoelastic fluids may comprise hyaluronic acid, chondroitin sulfate, cellulose, derivatives or mixtures thereof, or solutions thereof. In some variations, the viscoelastic fluid may comprise sodium hyaluronate. Additionally, or alternatively, the viscoelastic fluid may further include a drug. For example, the viscoelastic fluid may include a drug suitable for treating glaucoma, reducing or lowering intraocular pressure, reducing inflammation, fibrosis neovascularization or scarring, and/or preventing infection. For example, in some variations, the viscoelastic fluid may include the therapeutic agents described herein, such as but not limited to, Rho kinase (ROCK) inhibitors and agents for gene therapy, DNA, RNA, or stem cell-based approaches. The fluid compositions may include custom drug formulations.

### Elongate Member

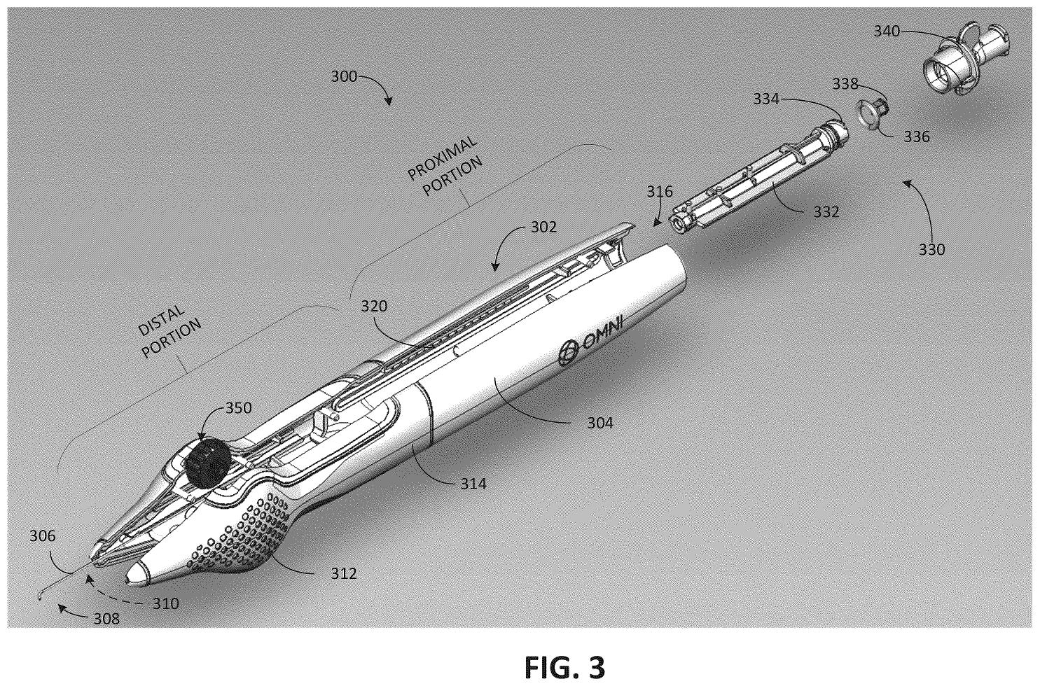

FIG. 3 depicts an exploded view of a variation of the delivery device. Some variations of the delivery devices described herein may comprise an elongate member (308) coaxially disposed within a cannula lumen (310). In some variations, the elongate member (308) may comprise a conduit defining a lumen and may be configured to deliver one or more fluid compositions and/or disrupt the trabecular meshwork or other similar tissues. In other variations, the elongate member (308) may be solid without a lumen but may still be configured to disrupt the trabecular meshwork or other similar tissues.

The elongate member (308) may be coaxially disposed and slidable within the cannula lumen (310) of the delivery systems described here. When the elongate member (308) is in a retracted position relative to the cannula (306), the distal end of the elongate member (308) may be located within (i.e., proximal to) a distal tip of the cannula (306). When the elongate member (308) is in an extended position relative to the cannula (306), the distal end of the elongate member (308) may be located outside of (i.e., distal to) the distal tip of the cannula (306). The length of extension of the elongate member (308) beyond the distal tip of the cannula (306) may correspond to the distance around Schlemm's canal that may be traversed by the elongate member (308) (e.g., in order to disrupt Schlemm's canal and/or surrounding trabeculocanalicular tissues, and/or to deliver a fluid composition). When a variation of the delivery systems described herein is used to deliver a fluid composition, the length traversed by the elongate member (308) may correspond to the length around Schlemm's canal to which the fluid composition is delivered. When a variation of the delivery systems described herein is used to mechanically tear or cut the trabecular meshwork independent of fluid delivery, the length traversed by the elongate member (308) may correspond to the length of trabecular meshwork that is cut or torn. In some variations, this length may be between about 1 mm and about 50 mm. In some of these variations, the length may be between about 10 mm and about 40 mm, between about 15 mm and about 25 mm, between about 16 mm and about 20 mm, between about 18 mm and about 20 mm, between about 19 mm and about 20 mm, between about 18 mm and about 22 mm, about 20 mm, between about 30 mm and about 50 mm, between about 35 mm and about 45 mm, between about 38 mm and about 40 mm, between about 39 mm and about 40 mm, or about 40 mm. The elongate member (308) may be moved between extended and retracted positions using a drive assembly of the delivery device, described in more detail herein.

The elongate member (308) may be sized so that it can be advanced through the cannula (306) and into a portion of Schlemm's canal (e.g., 0 to 360 degrees of the canal). In some instances, the outer diameter of the elongate member may be configured to disrupt trabeculocanalicular tissues, stent, and/or apply tension to the canal, and/or deliver a fluid composition. The elongate member (308) may be made from any suitable material that imparts the desired flexibility and pushability for introduction of the elongate member through the eye wall, accessing Schlemm's canal, and/or navigation through other ocular tissue structures. For example, the elongate member (308) may comprise a polymer; a polymer reinforced with a stiffening member such as, for example, a metal wire, braid or coil; composites of polymers and metal; or metals such as stainless steel, nickel, titanium, aluminum, shape-memory alloys (e.g., Nitinol), or alloys of any of the foregoing. Exemplary polymers include without limitation, polycarbonate, polyetheretherketone (PEEK), polyethylene, polypropylene, polyimide, polyamide, polysulfone, polyether block amide (PEBAX), fluoropolymers, and nylon.

To easily access Schlemm's canal, in some variations it may be advantageous to coat all or a portion of the elongate member (308) with a lubricious coating (e.g., lubricious polymer coating) to reduce friction between the body of the elongate member (308) and the cannula and/or the ocular tissue, as the elongate member (308) moves within the cannula and/or the ocular tissue. In some variations, the lubricious coating may be hydrophilic. In other variations, the lubricious coating may be hydrophobic. In some variations, additionally or alternatively, the elongate member (308) may be composed of one or more materials that have a lower coefficient of friction than the ocular tissues that the elongate member (308) contacts (e.g., the tissue of Schlemm's canal) when used as intended, and/or a lower coefficient of friction than the material of the cannula.

In variations in which the elongate member (308) is reusable, the elongate member (308) may be made from a material that can be sterilized (e.g., via autoclaving), such as a heat resistant metal (e.g., stainless steel, aluminum, titanium). The elongate member (308) may be straight with enough flexibility and pushability to navigate the ring-shaped Schlemm's canal or may be pre-shaped to about a 2-10 mm radius of curvature or about a 6 mm radius of curvature (i.e., the approximate radius of curvature of Schlemm's canal in an adult human) to more easily circumnavigate Schlemm's canal, partially or in its entirety. In some variations, the elongate member (308) may be configured to be advanced over or along a guidewire.

It may, in some variations, be desirable for the elongate member (308) to have one or more features to improve visualization of the elongate member (308) when the elongate member (308) is extended from the cannula (308). For example, the elongate member (308) may be colored (e.g., red, orange, yellow, green, blue, purple, etc.) and/or may have colored segments and/or distinguishable designs thereon spaced along the length of the elongate member (308). Additionally, or alternatively, visualization may be improved using an illuminated beacon, a fiber optic, side illuminating fiber optic, luminescence, fluorescence, or the like. For example, a fiber optic may travel along the body of the elongate member (308) to deliver light to the distal tip of the elongate member (308), which may improve visualization of the distal tip of the elongate member (308) as it is advanced or retracted about Schlemm's canal. Put differently, in some variations, a portion (e.g., distal end, central portion) of the elongate member (308) may be illuminated or may otherwise comprise an illumination device to assist in visualizing movement of the elongate member (308) within Schlemm's canal.

In some variations, the elongate member (308) may be sized to be advanced atraumatically (e.g., without trabecular meshwork disruption) through Schlemm's canal. In other variations, the elongate member (308) may be sized to have an outer diameter sufficient to disrupt Schlemm's canal and surrounding trabeculocanalicular tissues. The outer diameter may range from about 25 microns to about 1000 microns, from about 25 microns to about 500 microns, from about 50 microns to about 500 microns, from about 150 microns to about 500 microns, from about 200 microns to about 500 microns, from about 300 microns to about 500 microns, from about 200 microns to about 250 microns, from about 150 microns to about 200 microns, or from about 180 microns to about 300 microns. In some instances, it may be beneficial for the elongate member to have an outer diameter of about 240 microns, although portions (e.g., a distal tip) of the elongate member may differ in size and shape.

In some variations, the distal end of the elongate member (308) may be configured as a curved tip, a compound curved tip, an atraumatic tip, an enlarged atraumatic tip, or the like, to help the elongate member (308) advance through Schlemm's canal. In some of these variations, the distal end may comprise a blunt parasol-shaped atraumatic tip. In other variations, a distal portion of the elongate member (308) may optionally include a disruptive component, e.g., a notch, hook, barb, a rough surface, or combination thereof, to disrupt the juxtatrabecular portion of Schlemm's canal or juxtatrabecular meshwork. One or more projections emanating from the elongate member (308) may further disrupt the juxtatrabecular portion of Schlemm's canal or juxtatrabecular meshwork and thus increase permeability of aqueous humor through the trabecular meshwork into Schlemm's canal. In some instances, the elongate member (308) may also deliver energy to the trabeculocanalicular tissues (e.g., ultrasonic energy, radiofrequency energy (e.g., for electrocautery, electroablation), electromagnetic radiation, light energy (e.g., via a fiber optic)).

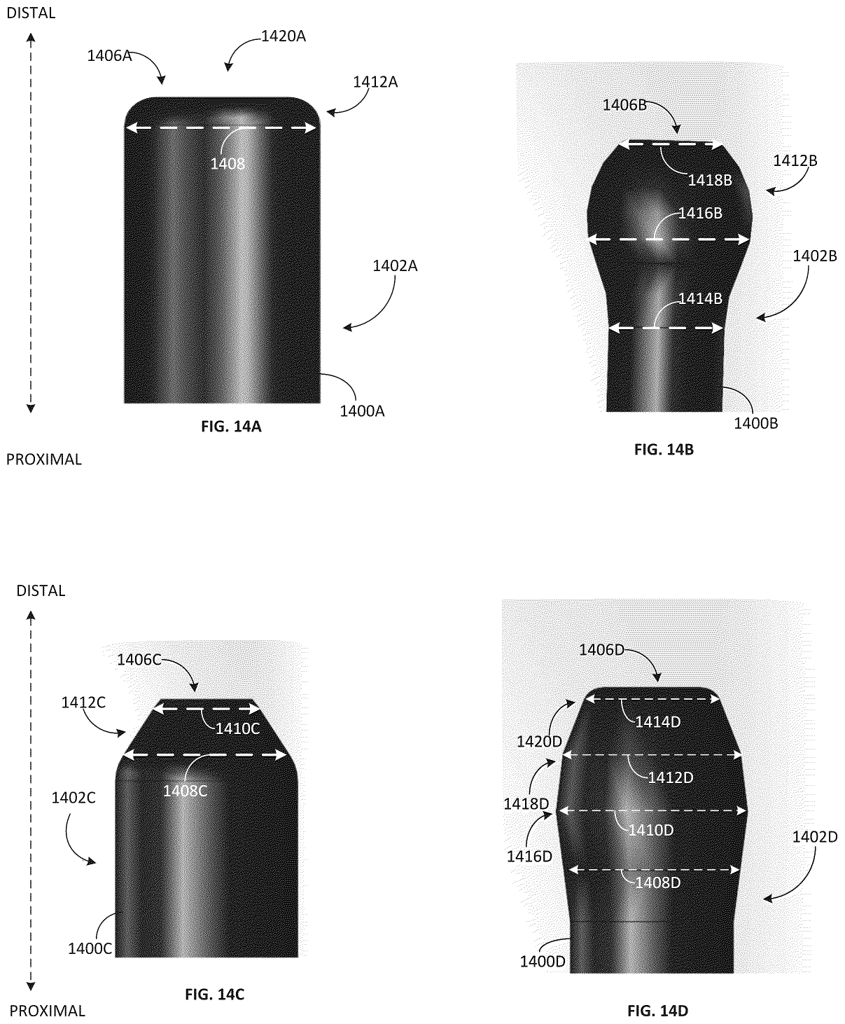

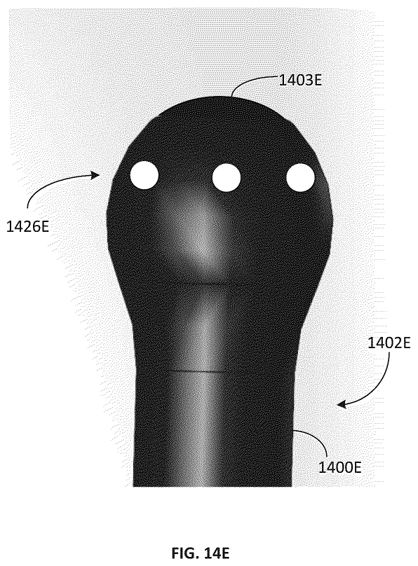

FIGS. 14A-14E depict various variations of the distal end of the elongate member. The variations depicted in FIGS. 14A-14E may facilitate access to, and advancement within, Schlemm's canal or other ocular tissue. For example, the configurations of the distal end of the elongate member depicted in FIGS. 14A-14E may assist in advancing the distal end of the elongate member through an opening formed in the trabecular meshwork (e.g., an otomy) to access Schlemm's canal, and/or may allow for easier advancement through Schlemm's canal once inserted therein.

As seen in FIG. 14A, the elongate member (1400A) may comprise a distal end (1402A) having a first length and a lumen opening (1406A) that is in fluid communication with a lumen (1420A) of the elongate member (1400A). The distal end (1402A) may have a constant outer diameter (1408A) along a first length, as illustrated in FIG. 14A. In some variations, the constant outer diameter (1408A) along the first length of the distal end (1402A) may be the same outer diameter as all or a portion of the remainder of the body of the elongate member (1400A), while in other variations, the outer diameter (1408A) of the distal end (1402A) of the elongate member (1400A) may be different than an outer diameter of a different portion of the body of the elongate member (1400A). In some variations, the distal end (1402A) may comprise a rounded distal edge (1412A).

In some variations, the distal end of the elongate member may not have a constant outer diameter along the length of the distal end. For example, as depicted in FIG. 14B, the distal end (1402B) may comprise a bulbous shape such that the outer diameter of the distal end (1402B) varies along the length of the distal end. In some variations, the bulbous shape may be at least partially spherical. The distal end (1402B) may comprise a proximal outer diameter (1414B), which may, in some variations, be the same as the outer diameter of the remainder of the body of the elongate member (1400B), a medial outer diameter (1416B), and a distal outer diameter (1418B). The medial outer diameter (1416B) may be larger than both the proximal outer diameter (1414B) and the distal outer diameter 1418B. The distal outer diameter (1418B) may correspond to the outer diameter of the distal most portion of the distal end (1402B) of the elongate member (1400B). In some variations, the proximal outer diameter (1414B) and the distal outer diameter (1418B) may be substantially the same diameter, while in other variations these outer diameters may be different. For example, the proximal outer diameter (1414B) may be smaller than the distal outer diameter (1418B), or the distal outer diameter (1418B) may be smaller than the proximal outer diameter (1414B). The distal end (1402B) of the elongate member (1400B) may also comprise a rounded distal edge.

In some variations, the distal end of the elongate member may comprise a taper terminating in the lumen opening. For example, as illustrated in FIG. 14C, the distal end (1402C) of the elongate member (1400C) may have a first outer diameter (1408C) in a first portion of the distal end (1402C) and a second, smaller, outer diameter (1410C) at a second portion of the distal end (1402C), the second portion being distal to the first portion. The outer diameter of the distal end (1402C) of the elongate member (1400C) may linearly decrease between the first and second portions such that the distal end (1402C) comprises a sloped surface. The first outer diameter (1408C) may be the same as, or different from (e.g., larger or smaller) than an outer diameter of the remainder of the body of the elongate body, while the second outer diameter (1410C) may generally be smaller than an outer diameter of the remainder of the body of the elongate body.

In some variations, the distal end may be configured as a combination of the configurations previously described herein. For example, the distal end may comprise one or more bulbous portions, one or more tapered or sloped portions, and/or a rounded distal edge. For example, the distal end may comprise a proximal sloped portion, a central bulbous portion, and a distal sloped portion, where the slope of the proximal portion is opposite the slope of the distal portion. In this manner, the outer diameter of the distal end at the proximal end of the proximal portion may be less than the maximum outer diameter of the bulbous portion, which may be greater than the outer diameter at the distal end of the distal portion. In another variation, the distal end may comprise a plurality of sloped portions (e.g., two, three, four, five or more) having different slopes. For example, FIG. 14D depicts an exemplary variation in which the distal end (1402D) comprises a plurality of sloped portions. As shown there, the distal end (1402D) may comprise a proximal sloped portion (1416D) having a positive slope, a central sloped portion (1418D) having a negative slope, and a distal sloped portion (1420D) have a negative slope different than the negative slope of the central portion (e.g., a larger negative slope). In this manner, the outer diameter of the distal end 1402D may vary (e.g., linearly) from a proximal outer diameter (1408D), to a first central outer diameter (1410D), to a second central outer diameter (1412D), and ultimately to a distal outer diameter (1414D), measured at the locations of slope change. The first central outer diameter (1410D) may be greater than the proximal outer diameter (1408D), the second central outer diameter (1412D) and the distal outer diameter (1414D). The second central outer diameter (1412D) may be greater than the proximal outer diameter (1408D) and the distal outer diameter (1414D). In some variations, the proximal outer diameter (1408D) and the distal outer diameter (1414D) may be about equal, while in other variations the proximal outer diameter (1408D) may be greater than the distal outer diameter (1414D). While described above having a central sloped portion having a negative slope, it should be appreciated that in some variations, this portion may not have a negative slope and may instead have a constant outer diameter.

In some variations, as seen in FIG. 14E, the distal end (1402E) of the elongate member (1400E) may comprise a rounded distal end (1403E). Additionally, the distal end (1402E) comprising the rounded distal end (1403E) may be advantageous for otomy access and tracking of the distal end (1402E) around Schlemm's canal. The rounded distal end (1403E) may be closed and may not include a single, central lumen opening. Instead, the distal end (1402E) may include one or more offset or side openings (1426E) in fluid communication with the lumen of the elongate member (1400E). In some variations, the distal end may include both a central lumen opening on the distal-most surface, and one or more offset or side openings, each of which may be in fluid communication with the lumen of the elongate member (1400E). In some variations, the one or more offset or side openings (1426E) may include a single offset or side opening, or a plurality of offset or side openings (e.g., two, three, four, five, six, seven, eight, nine, or more). The one or more offset or side openings (1426E) may be positioned in any suitable location on or around the distal end (1402E). For example, the openings may be aligned along a circumference of the distal end, may be offset along a circumference of the distal end (e.g., alternatingly positioned above and below the circumference), may be positioned in a plurality of rings around the distal end, may be randomly positioned on the distal end, or may have any other configuration suitable for fluid delivery. Each of the offset or side openings may be substantially the same size (e.g., comprise the same diameter) or may be different sizes (e.g., one or more offset or side openings may have different diameters from another of the offset or side openings). When the distal end (1402E) of the elongate member (1400E) is deployed within Schlemm's canal, the one or more offset or side openings (1426E) may direct the fluid composition exiting the lumen of the elongate member in the direction of the trabecular meshwork and/or in the opposing direction towards the collector channels.

Referring back to FIG. 3, as described above, the elongate member (308) may include a conduit comprising a lumen. The elongate member (308) may be configured to deliver a fluid composition. The fluid composition may travel through a lumen of the elongate member (308) and may be delivered through one or more openings of the lumen. The fluid composition may be used to disrupt the trabecular meshwork and other surrounding tissues.

In some variations, the distal end of the elongate member (308) may be configured or modified to aid delivery of the fluid composition into Schlemm's canal. For example, the distal end of the elongate member (308) may comprise a cut out configured as a half tube. Additionally, or alternatively to an opening at the distal tip, the elongate member (308) may optionally comprise a plurality of openings through its body (e.g., sidewall) that are spaced along the axial length of the elongate member (308). In this variation, the fluid composition may be delivered from the reservoir through the openings in the elongate member and into Schlemm's canal. This lateral ejection of the fluid composition (e.g., a viscoelastic fluid) may in some instances enhance disruption of outflow tissues and enhance permeability to aqueous humor. It is understood that the openings can be of any suitable number, size, and shape, and spaced along the axial length of the elongate member (including the distal tip) in any suitable manner.

### Drive Assembly

The delivery devices (300) may generally include a drive assembly (320) configured to move the elongate member (308) (e.g., conduit) and/or deliver a fluid composition into Schlemm's canal. The drive assembly (320) may be at least partially contained within the housing (304) and may include any suitable component or combination of components capable of providing the handle (302) with universal functionality, as depicted in FIG. 3.

The drive assembly may convert an external input (e.g., motion of a user's thumb or finger) into motion of one or more components of the delivery system. More specifically, the drive assembly may cause the slidable elongate member (308) (e.g., slidable conduit) to be extended distally out of the cannula (306), and/or it may cause the slidable elongate member (308) to be retracted proximally into the cannula (306). The drive assembly (320) may also optionally cause a fluid composition to be delivered from a fluid reservoir (332) through the elongate member (308) and/or cannula (306).

Each of these effects (i.e., extension of the slidable elongate member (308), retraction of the slidable elongate member (308), and/or delivery of a fluid composition), or any combination of these effects, may be actuated using the same actuator or a different actuator. Utilizing the same actuator may allow for easier and more precise single-handed use of the delivery system. For example, if the actuator comprises a rotatable element (such as the one or more wheels (350), as in variations described herein), rotating the rotatable element in a first direction may cause extension (e.g., advancement) of the slidable elongate member (308), and rotating the rotatable element in a second, opposite direction may cause retraction of the slidable elongate member (308). When the delivery system is configured to deliver a fluid composition, rotating the rotatable element (e.g., in the first and/or second direction) may also cause delivery of a fluid composition. The delivery of the fluid composition may be simultaneous with movement (e.g., advancement and/or retraction) of the slidable elongate member (308). In some of these instances, the fluid composition may be delivered to the portion of Schlemm's canal in which the slidable elongate member (308) is advanced. That is, the fluid composition may be delivered to the same arc length of Schlemm's canal as the extension of the elongate member (308). When delivery of the fluid composition is simultaneous with retraction of the elongate member (308), fluid composition may take the place of the slidable elongate member (308) as it is retracted and may dilate Schlemm's canal and/or the collector channels and/or the juxtacanalicular meshwork at that location in Schlemm's canal. Furthermore, the quantity of the fluid composition delivered may be tied to the amount of movement of the elongate member (308). That is, a certain predetermined, fixed volume of fluid composition may be delivered via the elongate member (308) (e.g., delivered out of the distal end of the elongate member (308)) for a fixed amount of movement of the elongate member (308) (e.g., a retraction distance) and for a fixed amount of rotation of the rotatable element.

In other variations, the quantity of the fluid composition delivered may not be tied to the amount of movement of the elongate member. Put differently, the quantity of the fluid composition delivered may be independent of the amount of movement of the elongate member. Accordingly, it can be appreciated that there can be two separate actuators for movement of the elongate member (308) and the delivery of the fluid composition. For example, a first actuator may be configured to advance and/or retract the elongate member (308) and a second actuator may be configured to deliver the fluid composition. If the first actuator comprises the rotatable element (e.g., the one or more wheels (350)), rotating the rotatable element in a first direction may cause advancement of the slidable elongate member (308), and rotating the rotatable element in a second, opposite direction may cause retraction of the slidable elongate member (308). When the delivery system is configured to deliver the fluid composition, the second actuator may be engaged to cause delivery of the fluid composition. The second actuator may include a slidable or translatable element (e.g., a slide), a rotatable element, a depressible element (e.g., a button), a lever, or the like (with or without mechanical advantage or mechanical disadvantage). It should be appreciated that while the foregoing actuators are described as rotatable, slidable or translatable, and depressible elements, any suitable actuators may be utilized, and any of these actuators may be used to cause advancement and/or retraction of the elongate member, and/or delivery of the fluid composition (e.g., the first actuator may be any of a slide, button, rotatable element or the like and the second actuator may be any of a slide, button, rotatable element, or the like).

In some variations, the drive mechanism (320) may be configured to allow the delivery system to be used only once—that is, the drive mechanism (320) may prevent, for example, re-advancement of the slidable elongate member (308) after a predetermined amount of extension and/or retraction. In other variations the drive mechanism (320) may be configured to allow the elongate member (308) to be advanced, retracted, re-advanced, and re-retracted an unlimited amount and number of times. Exemplary mechanisms by which external input may be converted into motion of one or more components of the delivery system are described in more detail herein.

In some variations, the drive assembly (320) may include components that translate rotational motion into linear motion. For example, the drive assembly (320) may include a linear gear and a pair of pinion gears. Each of the pinion gears may also be coupled to a rotatable component (350) (e.g., a wheel). In some variations, such coupling may be accomplished with a pin that can be threaded through a central opening in the rotatable component and pinion gear, and a nut that secures the rotatable component and pinion gear in a manner so that rotation of the rotatable component also rotates the pinion gear and vice versa. In some variations, the wheels may be attached to the pinion gear by one of the following methods: 1) the wheels and pinion gears are molded as one part using plastic injection molding technology; 2) the wheels slide onto the pinion gear and are secured with adhesive; or 3) the wheels slide on the pinion gear and are mechanically fixed with a fastener or a “press fit,” where the wheels are forced onto the pinion gear and friction holds them secure. The wheels and pinion gears may rotate coaxially, in the same direction, and at the same angular rate. In some variations, the wheel may have markings or colorings to indicate degree of advancement and/or direction of advancement.

One variation of the drive assembly (320) may comprise a linear gear, a pair of pinion gears, and at least one rotatable component coupled to each pinion gear. In other variations, the drive assembly may include a linear gear, a single pinion gear, and a single rotatable component coupled to the pinion gear. In variations with a pair of pinion gears, the pinion gears and associated wheel(s) may be disposed on either side of the linear gear. In some variations, the pinion gear(s) and linear gear may contact each other, i.e., the teeth of the pinion gears may directly engage corresponding teeth on the linear gear, and the wheels on one side of the linear gear may contact the wheels on the opposite side of the linear gear. In other variations, the pinion gear(s) and linear gear may be indirectly coupled, for example, via one or more idler gears.

At least a portion of the wheel (350) on a side of the linear gear may extend outside of the housing (304) for a user to manipulate. The drive assembly (320) may be manipulated with one hand when in a first configuration, and then manipulated with the same or the other hand when flipped over to a second configuration. A drive assembly (320) having such flexible capability can be easily used by someone who is right hand dominant or left hand dominant and may also be used in a procedure in which the handle is flipped during a procedure such that the cannula (306) is facing a first direction in a first portion of the procedure and facing a second direction in a second portion of the procedure. In a further variation, the drive assembly (320) may include one rotatable component on one side of the handle (302) and the “universal” feature of the handle (302) provided by the cannula (306) that itself can rotate instead of flipping the handle (302). When the wheel(s) and pinion gear(s) rotate coaxially in the same direction, and when there is no idler gear or an even number of idler gears (e.g., two) between the pinion gear(s) and the linear gear, distal rotation of the portion of the wheel(s) extending out of the housing may result in proximal translation of the linear gear within the housing (304), and conversely, proximal rotation of the portion of the wheel(s) extending out of the housing (304) may result in distal translation of the linear gear within the housing (304). When the wheel(s) and pinion gear(s) rotate coaxially in the same direction, and when there is an odd number of idler gears (e.g., one) between the pinion gear(s) and the linear gear within the housing, distal rotation of the portion of the wheel(s) extending out of the housing (304) may result in distal movement of the linear gear within the housing (304), and conversely, proximal rotation of the portion of the wheel(s) extending out of the housing (304) may result in proximal movement of the linear gear within the housing (304).

The drive assembly may also comprise one or more features to stabilize the pinion gears or otherwise keep them in place. For example, in some variations the drive assembly may comprise wheel spacers configured to sit between axles of the pinion gears.

In other variations, the rotational gears (i.e., pinion gears or idler gears) interfacing with the linear gear may be able to be disengaged from the linear gear by biasing their position off axis from the linear gear. This action de-couples the rotational gear teeth from the linear gear teeth to prevent linear gear movement with wheel rotation. The drive assembly may also be able to be locked to prevent rotation by engaging an intersecting pin or feature that prevents wheel rotation.

Further variations of the drive assembly may not employ translation of rotational motion to linear motion. For example, a slide (e.g., a finger slide) on the handle may be fixed or detachably coupled to a gear within the housing of the handle (e.g., a linear gear as previously described). Here the drive assembly may be configured so that advancement or retraction of the slide causes advancement or retraction of an elongate member and/or delivery of a fluid composition into Schlemm's canal. In some variations, the advancement or retraction of the slide may cause advancement or retraction of the elongate member while delivery of the fluid composition into Schlemm's canal may be actuated by a separate mechanism. In yet further variations, a button that can be pressed or squeezed may be employed instead of a slide, or a foot pedal may be employed to deliver a fluid composition, advance, and/or retract an elongate member. In some variations, the advancement or retraction of the elongate member may be actuated by a separate mechanism while the pressing or squeezing of the button may be employed to deliver the fluid composition to Schlemm's canal.

### Fluid Assembly

Some variations of the delivery devices (300), as depicted in FIG. 3, may include a fluid assembly (330) comprising one or more of: a fluid reservoir (332) in fluid communication with the elongate member (308) and the cannula (306), a sealing member (336), and a connector (340). The fluid assembly (330) may be at least partially contained within the housing (314) of the handle (304). For example, in some variations, the fluid reservoir (332) may be fully contained within the housing (314) of the handle (304), while in other variations, a portion of the fluid reservoir (332) may be contained within the housing (314) of the handle (304) and a portion may extend (e.g., proximally, laterally) beyond or outside of the housing (314) of the handle (304).

### Connector

The connector (340) may be configured to be releasably or detachably coupled to the fluid reservoir (330). A proximal region of the connector (340) may be configured to receive an end of an external fluid device to deliver the fluid composition to the fluid reservoir (332). Advantageously, the connector (340) may be removeable from the handle housing, simplifying, and streamlining fluid delivery to the fluid reservoir (332). In some variations, the connector (340) may be configured to couple with a coupling hub (334) at a proximal region of the fluid reservoir (332). In some variations, the fluid assembly (330) may further comprise a sealing member (336) and a plug (338). In variations in which the connector is removeable, the sealing member (336) may be configured to seal the fluid reservoir (332). The plug (338) may be configured to direct the fluid composition from the connector (340) into the fluid reservoir (332), as will be described in more detail herein.

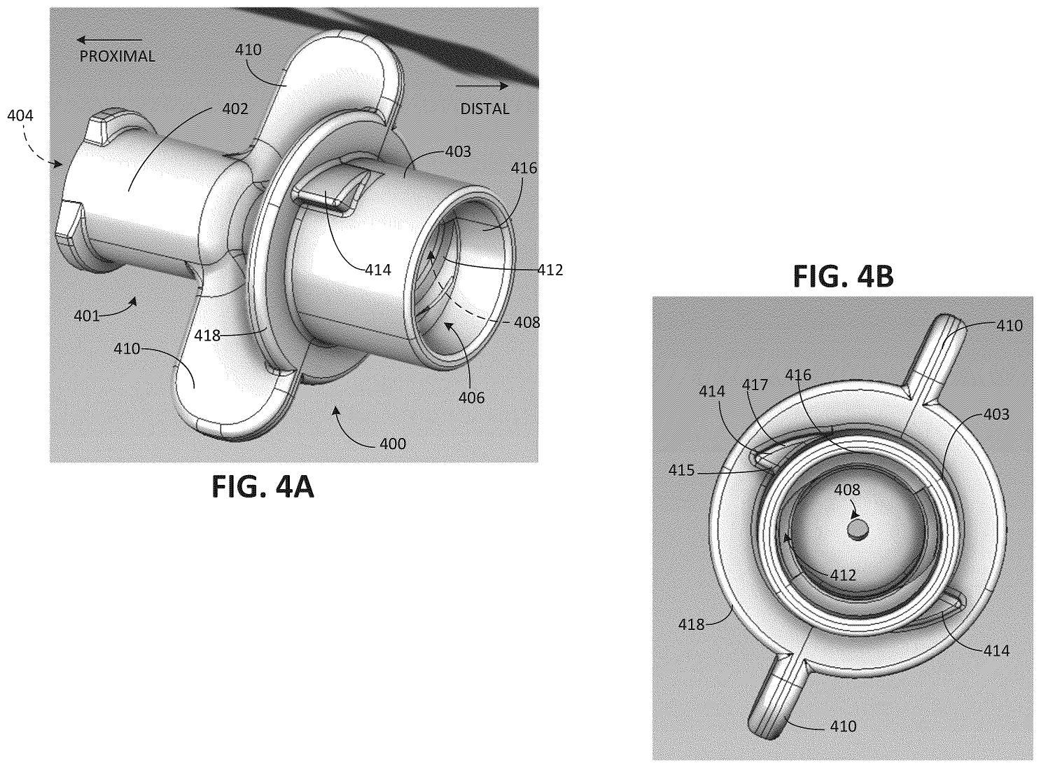

FIG. 4A illustrates a variation of a connector (400). As shown there, the connector (400) may comprise a connector body (401) having a proximal portion (402) with a proximal lumen opening (404), a distal portion (403) having a distal lumen opening (406) and a connector lumen (408) between and fluidly coupling the proximal lumen opening (404) and the distal lumen opening (406). The connector (400) may further comprise one or more tabs (410) (e.g., two, three, four, or more) extending radially outward from the connector body (401) (e.g., proximal and distal portions (402, 403)) and positioned between the proximal and distal portions (402, 403). When the connector (400) includes two or more tabs (410), a first tab may extend in a direction opposite a second tab, as illustrated in FIG. 4A. The one or more tabs (410) may be configured to be engaged by a user to grasp the connector (400) and easily disengage the connector (400) from the housing of the handle for removal. In some variations, the proximal portion (402), and in particular, the proximal lumen opening (404), may be configured to receive therein an end of the external fluid device to deliver the fluid composition through the connector (400) to the fluid reservoir. In other variations, the external fluid device may receive a portion of the connector, such a portion of the proximal portion (402) (e.g., the external fluid delivery device may fit over the proximal portion (402) and/or the proximal lumen opening (404)). The distal lumen opening (406) may define a distal luminal wall (416) that is configured to engage a coupling hub within the proximal cavity of the handle. The distal luminal wall (416) of the connector (400) may be configured to engage the coupling hub in any suitable manner, such as, for example, utilizing threads, a press fit, a snap fit, a bayonet, an interference fit, or the like.

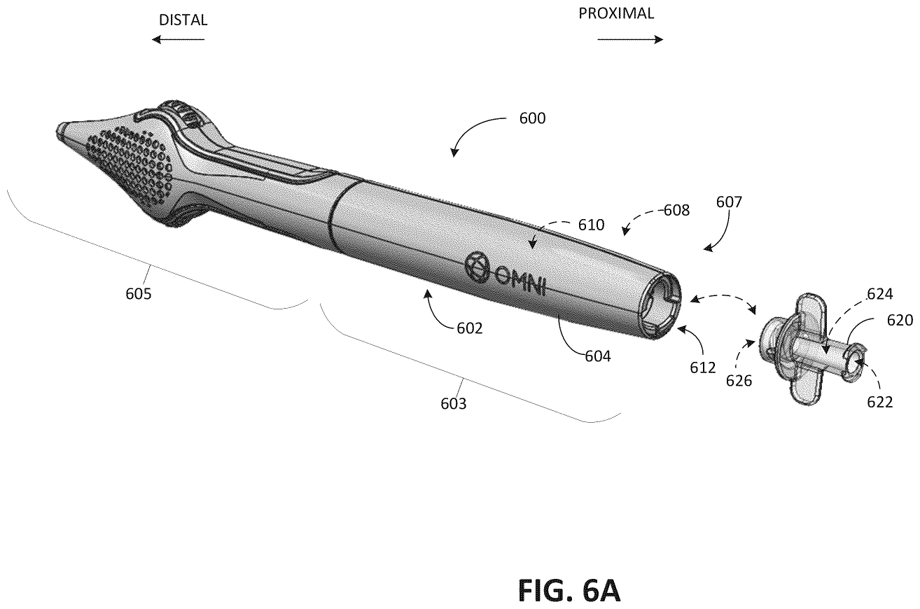

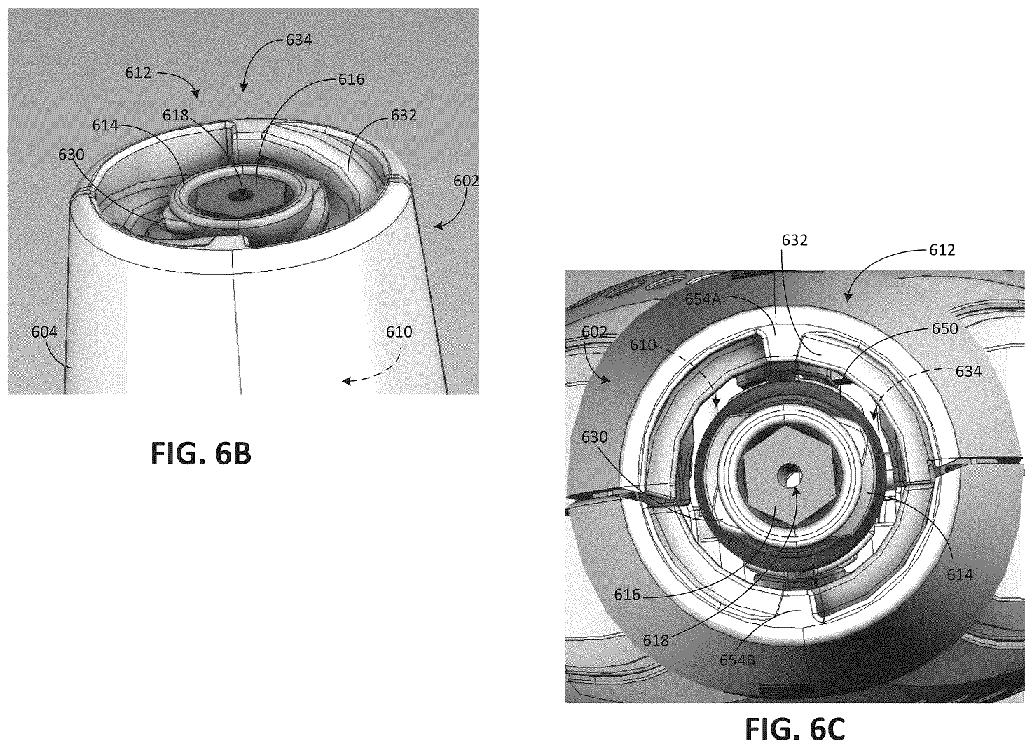

As illustrated in FIG. 4B, the distal luminal wall (416) may include one or more threads (412) configured to threadably engage corresponding threads of the coupling hub. The connector body (402) may include one or more extensions (414) configured to engage one or more abutments (654A/654B) of the proximal end of the handle to prevent rotational movement of the connector (400) (see FIG. 6C) when the connector (400) is coupled to the proximal opening of the handle (see FIG. 6A). The one or more extensions (414) may extend from the distal portion (403) and may comprise a flat surface (415) and a curved surface (417). In some variations, the flat surface (415) may extend a maximum distance from the distal portion (403) while the curved surface (417) may taper to a minimum distance from the distal portion (403). The flat surface (415) may physically contact the one or more abutments (654A/654B) when the connector (400) is rotated in a first direction. In variations containing two or more extensions (414) as depicted in FIG. 4B, the two or more extensions (414) may be oriented about 180 degrees apart. The one or more extensions (414) may properly orient the connector (400) within the proximal cavity of the handle by the one or more extensions (414) engaging with a threaded engagement (632) (see FIG. 6B). In some variations, the one or more extensions (414) engaging the one or more abutments (see FIG. 6C) may prevent the user from irreversibly securing the connector (400) to the connector hub of the handle (e.g., overtightening, cross threading, or the like). In some variations, the connector body (402) may further include a collar (418). The collar (418) may be configured as a stop, preventing the connector (400) from being advanced too deep into the proximal cavity of the handle. Put differently, the collar (418) may be configured to allow the connector (400) to be inserted into the proximal cavity of the handle to the appropriate depth. If the connector (400) is not inserted into the proximal cavity of the handle to the appropriate depth, proper fluid communication may not occur between the connector and the fluid reservoir, potentially leading to fluid pooling within the connector and introducing air into the fluid assembly. When the connector (400) is coupled to the coupling hub of the handle, the connector lumen (408) may be aligned with the plug lumen (see FIG. 6C) to allow fluid flow from the proximal lumen opening (404) of the connector (400) into the fluid reservoir in a consistent and timely manner. In some variations, the connector (400) may be pre-coupled to the handle, while in other variations, the connector (400) may be separate from the handle, allowing the user to couple the connector (400) to the handle at their convenience.

### Handle

The delivery systems described herein may include delivery devices comprising a handle capable of single-handed use by a single operator. The handle may be configured such that the ability to use the delivery system is independent of which hand a user chooses to use or on which eye a procedure is performed. For example, the handle may be configured for use in the left and right hands and for use on the left and right eyes. The handle may be further configured such that the ability to use the delivery system is independent of which direction around Schlemm's canal a tool and/or fluid composition is delivered. For example, the delivery system may be used to deliver a fluid composition in a clockwise direction in an eye, and then with a simple rotation of the handle (or by rotating the cannula itself 180 degrees in another variation) to a second orientation, may be used to deliver a fluid composition in the counterclockwise direction. However, it should be appreciated that in other variations, the delivery systems described herein may be configured to be used in a particular configuration (e.g., with a single side up, only in a clockwise direction, only in a counterclockwise direction, etc.).

Referring to FIG. 3, the handle generally includes a housing having a proximal portion comprising a proximal end, and a distal portion comprising a distal end and a grip portion proximal of the distal end. The proximal portion may generally be configured to contain, or at least partially contain, components of the fluid assembly (e.g., the reservoir). The distal portion, and more specifically, the grip portion, may generally be configured to be held by a user while positioning the cannula (e.g., advancing the cannula across the anterior chamber, puncturing the trabecular meshwork, advancing the cannula into Schlemm's canal), actuating the elongate member, and/or delivering the fluid composition. The proximal and distal portions of the housing may each generally include an interior cavity that may contain (or at least partially contain) internal components of the device, such as components of the drive assembly and fluid assembly. The distal portion (e.g., an interior cavity of the distal portion) may contain, or at least partially contain, components of the drive assembly and may house an internal portion of the cannula. It should be appreciated that one or more components of the drive assembly and/or the fluid reservoir may be configured to translate, and accordingly, may move between the internal cavities of the proximal and distal portions of the housing. In some variations, the distal portion of the housing (e.g., a distal end) may include a fluid port that may be configured to provide fluid for irrigation of the operative field and/or purge air from the system. The distal end of the housing may have the cannula coupled thereto. The grip portion of the distal portion may be raised, depressed, and/or grooved in certain areas, or otherwise textured to improve grasp of the handle by the user, increase the ergonomic fit of the handle into the hand of a user and control orientation of the handle without requiring wrist rotation, and/or to improve user comfort. The grip portion may be configured to allow the user to grip the handle close or adjacent to the cannula (e.g., within about 3 inches or less), while still allowing the user to actuate the elongate member and/or deliver the fluid composition via the one or more actuators. In some variations, the grip portion may be configured to allow the user to grip the handle within about 0.1-3 inches from a proximal end of the cannula. For example, the grip portion may be configured to allow the user to grip the handle within about 3 inches within about 2.5 inches, within about 2 inches, within about 1.5 inches, within about 1 inch, within about 0.75 inches, within about 0.5 inches, or within about 0.25 inches from a proximal end of the cannula. In some variations, the grip portion may be configured to allow the user to grip the handle within about 0.25-2 inches from the proximal end of the cannula. In some variations, the grip portion may be configured to allow the user to grip the handle within about 0.25-1.5 inches from the proximal end of the cannula. The proximal portion (e.g., a proximal end) may have a connector, such as the connector (400) depicted in FIGS. 4A and 4B, detachably coupled thereto. The connector may be configured to releasably couple to an external fluid device to transfer the fluid composition from the external fluid device into the fluid reservoir.

The handle or portions thereof (e.g., the grip portion) may be made from or may comprise any suitable material, including without limitation, fluoropolymers; thermoplastics such as polyetheretherketone, polyethylene, polyethylene terephthalate, polyurethane (or as thermoset), nylon, and the like; or silicone. In some variations, the housing or portions thereof may be made from or may comprise transparent materials. Materials with suitable transparency are typically polymers such as acrylic copolymers, acrylonitrile butadiene styrene (ABS), polycarbonate (PC), polystyrene, polyvinyl chloride (PVC), polyethylene terephthalate glycol (PETG), and styrene acrylonitrile (SAN). Acrylic copolymers that may be particular useful include, but are not limited to, polymethyl methacrylate (PMMA) copolymer and styrene methyl methacrylate (SMMA) copolymer (e.g., Zylar 631® acrylic copolymer). In variations in which the universal handle is reusable, the handle may be made from a material that can be sterilized (e.g., via autoclaving), such as a heat-resistant metal (e.g., stainless steel, aluminum, titanium) or a high-performance engineering polymer such as PEI, PEEK or PEKK.

The length of the universal handle may generally be between about 1 inch (2.5 cm) to about 20 inches (50.8 cm). In some variations, the length of the universal handle may be between about 4 inches (10.2 cm) and 10 inches (25.4 cm) from the distal end of the handle to the proximal end of the handle. In some variations, the length of the universal handle may be about 4 inches, about 4.5 inches, about 5 inches, about 5.5 inches, about 6 inches, about 6.5 inches or about 7 inches (17.8 cm) from the distal end to the proximal end of the handle.

The handle may be configured for ambidextrous use with an ergonomic fit in the hand. To that end, one or more of the proximal and distal portions of the handle may be configured to be symmetric across one or more planes (e.g., two planes), such as a YZ-plane and a XZ-plane. Such a configuration may make it easier for a user to rotate the handle (e.g., about 10 degrees to about 180 degrees or more) around a longitudinal axis of the handle during use. In some variations, such a configuration may make it easier for the user to rotate the handle about 15 degrees to about 30 degrees including about 20 degrees around the longitudinal axis of the handle during use of the device (e.g., during delivery of the fluid composition to each hemisphere of Schlemm's canal). In some variations, all portions of the handle may be symmetric across one or more planes (e.g., YZ-plane, XZ-plane, and a XY plane). In some variations, one or more of the distal portion and the proximal portion may be configured to be symmetric across one or more planes and may have a non-circular cross-sectional shape, while in other variations, one or more of the distal portion and the proximal portion may be configured to be symmetric across one or more planes and may have a circular cross-sectional shape. For example, in some variations, one or more portions may have a circular cross-sectional shape (e.g., the proximal portion, the distal end of the distal portion), and one or more portions may have a non-circular cross-sectional shape (e.g., the grip portion of the distal portion). It should be appreciated that the symmetric nature of the portions of the housing may refer only to the shape of the profile of that portion (e.g., outer surface) and may or may not include internal surfaces of the housing (i.e., the profile of the portion of the housing may be symmetric across a plane while the internal surface may include different pins, extensions, or other structures configured to interact with or engage internal components of the device).

### Distal Portion of the Handle

Referring to FIG. 2, the handle (200) may comprise a housing (206) further comprising a proximal portion (202) and a distal portion (201). In some variations, the distal portion (201) may comprise the grip portion (204). The grip portion (204) may comprise a first curved side and a second curved side opposite the first curved side, as will be described in more detail herein. In some variations, the grip portion (204) may comprise one or more actuators (210), wherein the one or more actuators (210) may be configured to move the elongate member (234) and/or deliver fluid. The grip portion (204) may further comprise a tapered region configured to receive fingers of the user. The tapered region may be distal to the one or more actuators (210) and/or the first and second curved sides. In some variations, the grip portion (204) may be commensurate in length with the distal portion (201), while in other variations, the grip portion (204) may be a segment or portion of the distal portion (201) such that a length of the grip portion (204) is less than a length of the distal portion (201). For example, in some variations, the grip portion and the distal portion may have the same proximal end, while the distal end of the grip portion may be proximal of the distal end of the distal portion. For example, in some variations, the distal end of the handle may not be part of the grip portion.

In some variations, the grip portion (204) may further comprise one or more flat regions (240) (e.g., two, three, four, or more). The flat regions (204) may be configured to receive a portion of a hand of the user (e.g., may be configured as a finger rest) during a procedure. In some variations, the one or more flat regions (240) may be proximal of the one or more actuators (210).

The one or more flat regions (240) may comprise a planar surface configured to provide a surface on which a user may rest a portion of the user's hand. In some variations, the grip portion (204) may include a first flat region comprising a first planar surface and a second flat region comprising a second planar surface, as will be described in more detail herein. The one or more flat regions may have any suitable size and shape. For example, in some variations, each of the one or more flat regions may be positioned across a majority of a width of a top surface or a bottom surface of the housing of the handle such that a majority of the top or bottom surface is planar. In some variations, the one or more flat regions may be positioned across about 50% to about 100% of the width of a top or bottom surface. For example, in some variations, the one or more flat regions may be positioned across at least about 50%, at least about 60%, at least about 70%, at least about 75%, at least about 80%, at least about 85%, at least about 90%, at least about 95%, or about 100% of the width of a top or bottom surface. In some variations, the one or more flat regions may have a maximum width that is about 50% to about 100% of a central width of the handle at a location aligned with the maximum width of the flat region. Each one of the one or more flat regions may have a maximum width that is at least about 50%, at least about 60%, at least about 70%, at least about 75%, at least about 80%, at least about 85%, at least about 90%, at least about 95%, or about 100% of a central width of the handle at a location aligned with the maximum width of the flat region.

Generally, the grip portion may a length equal to the total length of the distal portion or may be less than the total length of the distal portion. For example, in some variations, a ratio of the length of the grip portion to the length of the distal portion may be 1:1, 1:1.25, 1:1.5, 1:1.75, 1:2, 1:2.25, 1:2.5, 1:2.75, 1:3, 1:3.25, 1:3.5, 1:3.75, or 1:4.

In some variations, the grip portion (204) may comprise a non-slip material. The non-slip material may extend beyond the one or more actuators and may terminate adjacent to the distal end of the handle. In some variations, the grip portion (204) may comprise non-slip material on the tapered portion. For example, the non-slip material may surround a portion (e.g., the majority of) or all of the tapered portion. The non-slip material may circumferentially surround all or a portion of the tapered portion.

Generally, each of the proximal portion and the distal portion may have a length along a longitudinal axis, and the total length of the handle may be the sum of the lengths of the proximal and distal portions. In some variations, the proximal portion may have a length equal to the length of the distal portion, while in other variations the length of the proximal portion may be greater than, or less than, the length of the distal portion. For example, in some variations, a ratio of the length of the proximal portion to the length of the distal portion may be 1:1, 3:4, 2.5:4.5, 2:5, 1.5:5.5, 1:6 0.5:6.5, 4:3, 4.5:2.5, 5:2, 5.5:1.5, 6:1, or 6.5:0.5. In some variations, a ratio of the length of the proximal portion to the total length of the handle may be 1:2, 3:7, 2.5:7, 2:7, 1.5:7, 1:7, 4:7, 4.5:7, 5:7. In some variations, a ratio of the length of the distal portion to the total length of the handle may be 1:2, 3:7, 2.5:7, 2:7, 1.5:7, 1:7, 4:7, 4.5:7, 5:7.

In some variations, the proximal portion may have a length within the range of about 2 inches to about 7 inches and/or the distal portion may have a length within the range of about 2 inches to about 7 inches (including all values and sub-ranges therein). For example, the proximal portion may have a length within the range of about 3 inches to about 6 inches including about 4 inches to about 5 inches. The distal portion may have a length within the range of about 3 inches to about 6 inches including about 4 inches to about 5 inches.

Along the length of the handle, the proximal portion may have a first diameter and the distal portion may have a second diameter, different from the first diameter. In some variations, the first diameter and the second diameter may be equal. The first diameter and the second diameter may each be constant, or at least one of the first and second diameters (including both diameters) may vary along the length of the handle. In some instances, a first diameter (e.g., maximum diameter) of the proximal portion may be greater than a second diameter (e.g., maximum diameter) of the distal portion or a second diameter of the distal portion (e.g., diameter at a proximal end of the distal portion) may be greater than a diameter of the proximal portion (e.g., diameter at a proximal end of the proximal portion).

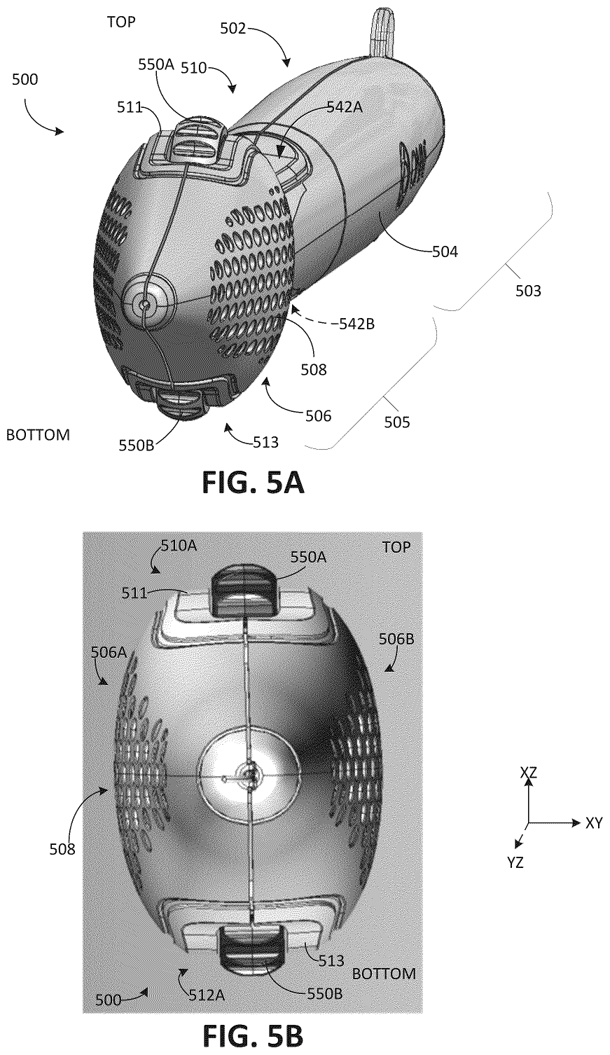

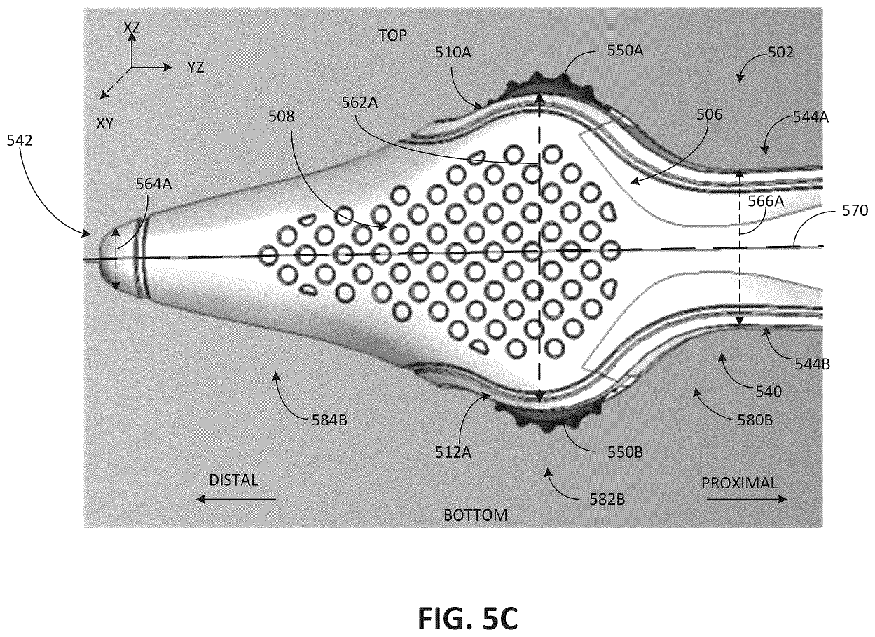

FIG. 5A depicts a delivery device (500) including a handle (502) comprising a housing (504) with a distal portion (505) and a proximal portion (503). The distal portion (505) of the housing (504) of the handle (502) may comprise a grip portion (506) configured to be received in a user's hand, or to otherwise be grasped or held during use of the device, and more specifically, while advancing the device to Schlemm's canal, accessing Schlemm's canal with the cannula, actuating the elongate member, and/or delivering a fluid composition. Although the user may grasp any location on the handle (502), including during transfer of the fluid composition into the fluid reservoir, the grip portion (506) may be particularly configured to receive a user's hand (e.g., fingers) while accessing Schlemm's canal and/or during delivery of the fluid composition to the eye, and may provide convenient access to the one or more actuators during a procedure. The grip portion (506) may be commensurate in length with the distal portion of the handle (502) or may form a portion or segment of the distal portion. In some variations, the grip portion (506) may be configured enable forward hand positioning of the user relative to the patient, while the proximal portion (503) may rest in the groove between a user's thumb and pointer finger. In general, the handle is designed to enable a user to securely grasp the grip portion close to cannula, wherein close to the cannula includes distal the one or more actuators. In some variations, the grip portion (506) may taper towards the cannula. thereby forming an elongated nose, which may further allow for precise control of the distal portion by providing a surface that easily and comfortably allows for placement of a user's finger(s). Moreover, the grip portion (506) is configured to facilitate the user grasping and maneuvering the device close to the cannula, which may optimize a user's control of the cannula during use (e.g., while accessing Schlemm's canal). The position and shape of the grip portion (506) may also bring the hand of the user closer to the patient, which may improve stability and control of the device. Moreover, the handle, and in particular, the grip portion (506) of the handle, may be configured to provide points of contact for steering and control, as described above, while also allowing a user to access, without repositioning of the hand, the one or more actuators of the device.

As mentioned above, generally, the handle may be configured such that a user may grasp the handle at the grip portion and still easily access the one or more actuators that may be used to actuate the elongate member and/or deliver the fluid composition to Schlemm's canal. As depicted in FIG. 5A, the one or more actuators may include two actuators (550A/550B), a first actuator (550A) on a top side and a second actuator (550B) on a bottom side.

In some variations, the device may be configured such that a user may grip the handle at the grip portion (506) to deliver a fluid composition in a clockwise direction in the eye and then with a simple rotation of the handle (502) (or by rotating the cannula itself 180 degrees in another variation), the device may deliver the fluid composition in the counterclockwise direction in the eye. In some variations, as described above, the grip portion (506) may comprise one or more flat regions proximal to the one or more actuators (550A/550B). Each of the one or more flat regions may comprise a planar surface, as seen in FIG. 5A. In some variations, the grip portion (506) may comprise a first flat region (542A) further comprising a first planar surface on the top of a handle (as seen in FIG. 5A). The grip portion (506) may further comprise a second flat region (542B) further comprising a second planar surface on the top of the handle. In some variations in which the grip portion comprises a first flat region and a second flat region, the first flat region and the second flat region may have corresponding sizes and/or shapes, and/or may be symmetric with one another across a central longitudinal axis of the handle.

The grip portion (506) of the handle may be configured to be non-slip and/or at least partially compressible relative to the remainder of the handle. For example, the grip portion (506) may have characteristics that make it easier to hold relative to the remainder of the handle, such as for example, comprise material with a higher coefficient of friction and/or a lower durometer than the remainder of (e.g., the proximal portion, a proximal end of the distal portion) the housing of the handle. The grip portion (506) may comprise a plurality of materials, including for example, a material with the above-mentioned characteristics overlaid or otherwise covering the material from which the remainder of the housing is formed. For example, in some variations, the housing may be formed of a first material, such as ABS or PC, and the grip portion may further comprise a second material, such as an elastomer (e.g., rubber) coupled to the first material (e.g., an external surface thereof). In some variations, the proximal portion (503) may be formed of a first material, the distal portion (505) may be formed a second material different from the first material, and the grip portion (506) may be formed of a third material different from the first material and the second material. In some variations, the increased friction between the grip portion (506) and the hand of the user due to material of the grip portion having a higher coefficient of friction compared to other materials of the handle allows the user to maintain control and/or orientation of the device without the device slipping out of the hand of the user.

In some variations, the grip portion (506) or a portion thereof may include a textured surface (508) configured to help the user retain contact and maneuver the handle (502). The textured surface (508) may be overlaid on the grip portion (506) and be comprised of a different material than the grip portion (506). In some variations, the textured surface (508) may extend from the distal end of the distal portion to the proximal portion. The textured surface (508) may include raised elements (e.g., protrusions) and/or indented elements (e.g., impressions, imprints, engraving, etc.), or a combination thereof. For example, raised elements may include bumps and/or indented elements may include circular indentations. Each of the raised or indented elements may be the same size or different sizes (e.g., cross sectional area, diameter, etc.). Each of the raised or indented elements may be the same shape (e.g., triangular, circular, ovular, square, rectangular, hexagonal, octagonal, or the like) or may be different shapes. The textured surface (508) may have a constant pattern across the grip portion (506) or may have a varied pattern configured to enhance tactile feel of the textured surface (508) in hand. For example, in an embodiment, the textured surface (508) may have protrusions of a first height closer to the one or more actuators (550) to help the user rotate the grip portion (506), while the textured surface (508) may have protrusions of a second, different height further away from the one or more actuators (550). In some variations, the first height may be greater than the second height, while in other variations the second height may be greater than the first height. In another embodiment, the textured surface (508) may have indentations of the same shape with a greater cross-sectional area closer to the one or more actuators (550) to increase tactile feel closer to the one or more actuators (550) while the indentations further away from the one or more actuators (550) have a small cross-sectional area.

Each of the top surface (510) and the bottom surface (513) may comprise features that assist a user in identifying, via touch (e.g., without visually identifying), the location of one or more actuators of the device. For example, in some variations, the top surface (510) and/or the bottom surface (513) may comprise an actuator border (511, 513). The actuator borders (511, 513) may be comprised of a different material than the grip portion (506) or may be comprised of the same material as the grip portion (506). In some variations, the actuator borders (511, 513) may include indented elements or raised elements, allowing a user to clearly identify through touch the boundaries of the actuator borders (511,513) and the actuators (550A, 550B). In some variations, one or more (e.g., both) of the actuator borders may be contiguous with the planar surface of a corresponding flat region.

The grip portion (506) may be shaped in a way to ergonomically fit into a hand of the user. To this end, in some variations, the grip portion (506) may be symmetric across a YZ plane, as depicted in FIG. 5B. The YZ plane may divide the grip portion (506) into a first grip portion or half (506A) and a second grip portion or half (506B) that are symmetric across the YZ plane. The first and second grip portions (506A/506B) may each comprise a rounded profile from a first, top surface (510) to a second, bottom surface (512) opposite the top surface (510). In this manner, the grip portion (506) may comprise a first curved side and a second curved side opposite the first curved side. More specifically, the first grip portion (506A) may comprise the first curved side and the second grip portion (506B) may comprise the second curved side. In some variations, the first and second grip portions (e.g., the first and second curved sides) may be symmetric across the cannula. In some variations, the first grip portion (506A) and the second grip portion (506B) may be symmetric across the XZ plane. In some variations, a portion of each of the first curved side and the second curved side may align with the first and second the actuators (550A/550B), respectively. For example, in some variations, the first and/or second actuator may be longitudinally centered along the first and/or second curved side, respectively.

In some variations, the first grip portion (506A) (e.g., first curved side) and the second grip portion (506B) (e.g., second curved side) may each be convex or comprise a convex curve. In some variations, the convex curve of the first grip portion (506A) and the second grip portion (506B) may have a radius of curvature of about 0.3 inches to about 1.5 inches. In some variations, the radius of curvature of the convex curve of the first grip portion (506A) and the second grip portion (506B) may include ranges of about ⅜ inch to about 1.5 inches, about ½ inch to about 1.5 inches, about ¾ inch to about 1.5 inches, about 1 inch to about 1.5 inches, and about 1.25 inches to about 1.5 inches. In some variations, the radius of curvature of the convex curve of the first grip portion (506A) and the second grip portion (506B) may include ranges of about 0.3 inch to about 1 inch, about ⅜ inch to about ¾ inch include about ½ inch. The radius of curvature of the convex curve of the first grip portion (506A) and the second grip portion (506B) may include ranges of about 0.3 inch to about 1.5 inches, about 0.3 to about 1.25 inches, about 0.3 inch to about 1.0 inch, about 0.3 inch to about ¾ inch, about 0.3 inch to about ½ inch, and about 0.3 inch to about ⅜ inch. In some variations, the first grip portion (506A) may have a first arc (e.g., a convex arc) with a first center, the second grip portion (506B) may have a second arc (e.g., a convex arc) with a second center, and the first and second centers may be on opposing sides of a central longitudinal axis of the cannula and/or a central longitudinal axis of the handle, as seen in FIG. 5B. In some variations, utilizing convex first and second grip portions (506A, 506B) may allow the user to re-orient or slightly rotate the grip portion between a thumb and one or more opposing fingers, rather than using the wrist to re-orient or rotate the entire handle. This provides an ergonomic benefit to the user and may help reduce arm fatigue. Furthermore, slight rotational movement of the first grip portion (506A) and the second grip portion (506B) may translate into movement of the distal tip. For example, in some variations, the first grip portion (506A) and the second grip portion (506B) may allow a limit of orientation by a user's grip alone to be about +/−30 degrees about a central longitudinal axis, without the user having to rotate at the wrist.

The first and second grip portions (506A, 506B) (e.g., first and second curved sides) may each have a one or more radii of curvature between the top and bottom surfaces (510A, 512A). For example, the first and second grip portions (506A, 506B) may each comprise a first, smaller radius of curvature near or adjacent the top surfaces (510A/512A) and a second, larger radius of curvature between (e.g., midway) the top and bottom surfaces (510A/512A), allowing the grip portion (506) to ergonomically fit into the hand of the user, contacting the hand of the user at or along multiple points to increase control of the device. In some variations, one or more of the first and second grip portions (506A/506B) may each comprise faceted faces (e.g., polygonal comprising two or more faces) from the first, top surface (510) to the second, bottom surface (512) opposite the top surface (510). In some variations, the faceted faces may collectively comprise a curve having a radius of curvature within the ranges of radius of curvature described herein. The faceted faces of one or more of the first and second grip portion (506A/506B) may be configured to control an angle of rotation of the grip portion within the hand of the user. It should be appreciated that while described in the preceding two paragraphs with respect to the first and second grip portions (506A, 506B), such features are also applicable to the first and second curved sides that may form, in some variations, the first and second grip portions respectively.

As illustrated in FIG. 5B, each of the actuators (550A/550B) may be positioned on or may otherwise extend from the top and/or bottom surfaces (510A, 512A) of the grip portion. The actuators (550A/550B) may extend a defined distance from the top and bottom surfaces (510A/512A) and/or may have a distinct shape so that the actuators (550A/550B) are easily distinguishable from each of the surfaces (510A/512A) themselves when the user is handling the grip portion (506). The top and bottom surfaces (510A/512A) may be substantially flat or may otherwise have a large radius of curvature relative to other portions of the handle (e.g., grip portion, neck, proximal portion, thus allowing a user to easily rest a finger on the top and bottom surfaces (510A/512A), placing the finger close to but not on the actuators (550A/550B). Referring to FIG. 5C, the top surface may include a first flat region (544A) and the bottom surface may include a second flat region (544B). The user may rest one or more fingers on each of the first flat region (544A) and/or second flat region (544B).

In designing the handle (502) to have enhanced ergonomic features, the grip portion (506) may be symmetric across an XZ plane parallel to a longitudinal midpoint (570), as illustrated in FIG. 5C. The symmetry of the grip portion (506) across the XZ plane allows the user to grip the top surface (510) with substantially the same orientation as the bottom surface (512). Furthermore, the symmetry of the grip portion (506) allows the user a preference in using either the first actuator (550A), the second actuator (550B), or both in actuating the elongate member and/or delivering the fluid composition.

When a user's thumb or finger is contacting either the top surface (510) or the first actuator (550A) or the bottom surface (512) or the second actuator (550B) (e.g., including one or more flat regions (544A/544B)), the textured surface (508) of the grip portion (506) may contact multiple locations on the user's hand for stability of the delivery device (500) during delivery of the fluid composition to the eye.

The grip portion (506) may have an hourglass shape, as illustrated in FIG. 5C. The grip portion (506) may taper from the top surface (510A) to a distal end (542) of the grip portion (506). The grip portion (506) may have maximum height (562A) at the location along the longitudinal axis having the first actuator (550A) and the second actuator (550B). The grip portion (506) may have a minimum height (564A) at the distal end (542). The tapering of the grip portion (506) from the maximum height (562A) to the minimum height (564A) may provide the grip portion (506) with a compact feel within the hand of the user, allowing the user to easily rotate the handle (502) around a longitudinal axis to rotate the cannula as needed. Aligning the actuators (550A/550B) with the portion of the grip portion that has the maximum height (562A) may allow for the greatest separation between the two actuators (550A/550B), thus allowing the user to clearly distinguish between the two actuators (550A/550B) while still being able to control each actuator individually or simultaneously. In some variations, the maximum height (562A) may be aligned with at least a portion of the actuators (550A/550B). For example, in an embodiment, the maximum height (562A) may be aligned with a midpoint of each of the actuators (550A/550B).

The height of the grip portion (506) may vary (e.g., decrease) proximally from the maximum height (562A) at the one or more actuators (550A/550B) to a smaller height (566A) at the neck (540) positioned proximally of the one or more actuators (550A/550B). In some variations, the rate of change from the maximum height (562A) at the one or more actuators (550A/550B) to the height (556A) at the neck (540) may be greater than the rate of change of the taper from the maximum height (562A) at the one or more actuators (550A/550B) to the minimum height (564A) at the distal end (542). In some variations, the maximum height (562A) may be a height of about 0.8 inches to about 2.0 inches. For example, in some variations, the maximum height (562A) may be about 0.8 inches, about 0.9 inches, about 1.0 inches, about 1.1 inches, about 1.2 inches, about 1.3 inches, about 1.4 inches, about 1.5 inches, about 1.6 inches, about 1.7 inches, about 1.8 inches, about 1.9 inches, or about 2.0 inches. In some variations, the height (566A) of the neck (540) may be a height of about 0.2 inches to about 0.8 inches. For example, in some variations, the height (566A) of the neck (540) may be about 0.2 inches, about 0.3 inches, about 0.4 inches, about 0.5 inches, about 0.6 inches, about 0.7 inches, or about 0.8 inches. In some variations, the minimum height (564A) may be about 0.05 inches to about 0.4 inches. For example, in some variations, the minimum height (564A) may be about 0.05 inches, about 0.1 inches, about 0.15 inches, about 0.2 inches, about 0.25 inches, about 0.3 inches, about 0.35 inches, or about 0.4 inches. In some variations, the ratio of the maximum height (562A) to the height (566A) of the neck may be about 5:1, about 4.5: 1, about 4:1, about 3.5:1, about 3:1, about 2.5:1, about 2:1, or about 1.5:1. In some variations, the ratio of the maximum height (562A) to the minimum height (564A) may be about 16:1, about 17:1, about 18:1, about 19:1, about 20:1, about 25:1, about 30:1, about 35:1, or about 40:1. In some variations, the ratio of the height (566A) of the neck to the minimum height (564A) may be about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1.

In some variations, a bottom of the grip portion (506) may comprise a first curved surface (580B), a straight surface (584B) with a second curved surface (582B) therebetween. The first curved surface (580B) may be proximal of the second curved surface (582B), and the first curved surface (580B) may be concave while the second curved surface (582B) may be convex. The second curved surface (582B) may include the actuator (550B). With the grip portion (506) being symmetric across the XZ plane, it can be appreciated that the top surface (510) may include corresponding curved and straight surfaces. The straight surface (584B) may be distal to the second curved surface (582B), and the straight surface (584B) may be tapered along the longitudinal axis until the distal end (542).

In some variations, the grip portion (506) may comprise different cross-sectional shapes along the longitudinal axis of the handle. For example, proximally, the grip portion (506) may comprise a circular cross-sectional shape with a first diameter while a central portion of the grip portion (506) comprising the top and bottom surfaces (510A/512A) and the actuators (550A/550B) may comprise an oblong or oval cross-sectional shape with a major axis and a minor axis, and a distal portion (including the distal end) of the grip portion (506) may comprise a circular cross-sectional shape with a second diameter. The major and minor axes may increase distally from the proximal portion of the grip portion (506) comprising the circular cross-sectional shape to a maximum major axis and a maximum minor axis, which may be aligned with the location of the actuators (550A/550B), and then may decrease distally until the distal portion of the grip portion (506) comprising a circular cross-sectional shape with the second diameter. In some variations, the first diameter may be larger than the second diameter and the maximum major axis may be larger than both the first diameter and the second diameter. In some variations, the major axis may be at least about 1.5 times to at least about 3 times the minor axis. In some variations, the major axis may be at least about 1.5 times to at least about 2.5 times the diameter of the proximal portion of the handle. The first diameter of the grip portion may be substantially equal to the diameter of the proximal portion of the handle. In some variations, the diameter of the proximal portion of the handle may be greater than the first diameter of the grip portion. In some variations, the difference in the diameters of the different cross-sectional shapes along the longitudinal axis of the handle may provide added benefits. For example, in some variations, the smaller the diameter of the handle, the more degrees of rotation may occur per movement, especially at the distal end.

As noted above, in some variations, the grip portion may comprise different cross-sectional shapes along the longitudinal axis of the handle. In some variations, one or more of the cross-sectional shapes may include a polygonal shape. For example, proximally, the grip portion (506) may comprise a circular cross-sectional shape with a first diameter while the central portion of the grip portion (506) may comprise a polygonal shape having faceted faces (e.g., two or more faces), and the distal end of the grip portion (506) may comprise a circular cross-sectional shape. The polygonal shape may include a triangular prism, a rectangular prism, a pentagonal prism, a hexagonal prism, a heptagonal prism, an octagonal prism, or the like. In some variations, the faceted faces may collectively comprise a radius of curvature. In some variations, the cross-sectional shape of the housing of the handle at the one or more flat regions may be different from the cross-sectional shape at any of: the distal end of the distal portion, the neck, or the proximal portion.

The shape of the handle, the differences in the diameters of the portions of the handle along the longitudinal axis, and the taper of the grip portion may assist in functionally balancing the intentional movement needed to rotate the distal end of the device with the precise control over subtle movement needed during use.

Moreover, the handles described herein may be configured to promote or otherwise facilitate a forward grip (i.e., more distal on the handle), which may provide the user with added control over the cannula. For example, the grip portion of the handle may include a taper (e.g., an elongated nose), as depicted in FIG. 5C. As shown there, the grip portion (506) may comprise a taper from the maximum height (562A) to the minimum height (564A) of the grip portion (506). The taper in the distal portion may promote a forward grip of the handle by the user (i.e., a grip of the distal portion on the handle). Advantageously, a grip of the user located more distally on the handle places the hand of the user in closer proximity to the eye of the patient, allowing the user more control over the cannula. Furthermore, the taper from each of the top and bottom surfaces (510A/512A) to the distal end (542) provides a location (e.g., the grip portion (506)) for a user to place an index finger distal the actuators (550A/550B). The user may seamlessly move their index finger or thumb between the grip portion (506) and the actuators (550A/550B) to access Schlemm's canal (e.g., advance the device to Schlemm's canal, puncture the trabecular meshwork, advance the distal tip of the cannula into Schlemm's canal), actuate the elongate member and/or deliver the fluid composition. Additionally, the taper from the maximum height (562A) to the minimum height (564A) of the grip portion (506) provides multiple resting points for fingers of the user, which may additionally assist in reducing user fatigue.

### Proximal End of the Handle

FIG. 6A depicts a perspective view of a delivery device (600) including the handle (602) having a distal portion (605) and a proximal portion (603) having a proximal end (607). The proximal portion (603) of the handle may comprise a circular cross-sectional shape having a diameter. In some variations, the proximal portion (603) of the handle (602) may have a constant diameter along the longitudinal axis, or the diameter may vary along the longitudinal axis (e.g., the diameter may taper to a smaller diameter at the proximal end (607)). In some variations, the proximal portion (603) may have an oblong or oval cross-sectional shape with a major axis and a minor axis. The proximal end (607) may include a connector (620) configured to couple to (e.g., receive) an external delivery device to transfer the fluid composition to a reservoir of the delivery device (600). The connector (620) may be configured to seamlessly facilitate transfer of the fluid composition from outside the delivery device (600) to the fluid assembly (608) of the housing (604) of the handle (602), while purging any air contained within the fluid assembly. As shown in FIG. 6A, the housing (604) of the handle (602) of the delivery device (600) may include an opening (612) at a proximal end configured to at least partially receive the connector (620) therein. Advantageously, having the connector (620) that detachably couples to the fluid assembly (608) at the proximal opening (612) of the housing (604) allows a user to transfer the necessary volume of the fluid composition to the fluid reservoir of the delivery device easily and quickly. Additionally, or alternatively, in some variations, the delivery device may be configured to automatically seal fluid within the fluid reservoir upon removal of the connector (620). Removal of the connector may allow the handle (602) to remain compact, without the connector (620) extending from the housing (604), during use. In some variations, the fluid reservoir may include one or more engagements to facilitate the coupling of the connector to the handle. For example, the fluid reservoir may include a threaded engagement to allow the connector to couple thereto. In some variations, the fluid reservoir may include other engagements to allow the connector to couple thereto without the need for rotational movement. For example, the fluid reservoir may include a snap fitting, a bayonet fitting, an interference fitting, or the like.

The connector (620) may be configured to connect to the housing (602) via the proximal opening (612). The connector (620) may have a connector lumen (624) therethrough. The connector lumen (624) may be in fluid communication with, and may terminate at one end in, a proximal lumen opening (622) and at the other end, in a distal lumen opening (626). When the connector (620) is coupled to the delivery device (600) at the opening (612), the connector lumen (624) may provide fluid communication between the proximal lumen opening (622) and the fluid reservoir (610). The external fluid device may be coupled to the connector (620), for example, a portion (e.g., distal end) of the external fluid device may be placed into the proximal lumen opening (622) to deliver the fluid composition to the fluid reservoir (610). Once a desired volume of fluid has been delivered to the fluid reservoir (610) from the external fluid device, the connector (620), and in some variations, the connector (620) coupled to the external fluid device, may then be removed from the delivery device (600), allowing the user to deliver the fluid composition to the eye, as will be described in more detail herein.

It should be appreciated that the connector (620) may be connected to and unconnected to the housing via several mechanisms. For example, the connector may rotatably couple to the housing or may axially couple to the housing (without rotation). For example, in variations in which the connector axially couples to the housing, the connector may comprise a snap fit connector, a bayonet fitting, and/or any other connector suitable for use in an axial connection. FIG. 6B illustrates a perspective view of the proximal opening (612) of the housing (604). As described above, the proximal opening (612) may be configured to releasably couple the connector to the housing (604) of the handle (602). The handle (602) may include a proximal cavity (634), which may be at least partially defined by the proximal opening (612). The proximal cavity (634) may receive the connector therein. In some variations, the housing (604) may include a coupling hub (614) having a plug (616) positioned therein. The coupling hub (614) may proximally extend into the proximal cavity (634). The plug (616) may have any suitable cross-sectional shape, and may be, for example, hexagonal, circular, square, ovular, etc. The plug (616) may have a plug lumen (618), which may be in fluid communication with the fluid reservoir (610). The plug (616) and the plug lumen (618) may be configured to direct the fluid composition being transferred through the connector to the fluid reservoir. In some variations, the plug (616) retains a valve by trapping a sealing member (650) in a proximal bore of the fluid reservoir (610).

As illustrated in FIG. 6B, the coupling hub (614) may be configured to threadably engage the connector (see FIG. 6A). In some variations, the coupling hub (614) may include a coupling portion (630) configured to mate with a corresponding portion of the connector. The coupling portion (630) may include a mechanical coupling, such as for example, threads. In other variations, the coupling portion (630) may include other mechanical coupling features (e.g., a press fit, an interference fit, a snap fit, a magnetic fit, or the like). In some variations, the mechanical coupling (e.g., the threads) may extend radially outward from the coupling hub (614) into the proximal cavity (634), as depicted in FIG. 6B. In some variations, the housing (604) may include a threaded engagement (632) extending inward into the proximal cavity (634). In some variations, the threaded engagement (632) may be sloped distally to allow the connector to be rotated in the distal direction, into the proximal cavity (634). This threaded engagement (632) may be configured to facilitate the rotational movement of connector tabs, as will be described herein.

FIG. 6C illustrates a rear view of the handle (602) including the proximal opening (612) and the proximal cavity (634). The proximal cavity (634) may comprise additional components to allow the connector to transfer the fluid composition to the fluid reservoir (610). For example, a sealing member (650) may be positioned within the proximal cavity (634) and may be configured to seal the fluid reservoir (610) distal the coupling hub (614). The sealing member (650) may include an O-ring comprised of any elastomeric material. In some variations, the sealing member (650) may be configured to help direct the fluid composition into the fluid reservoir (610).

In some variations, the fluid reservoir (610) may include a valve distal to the sealing member (650). The valve may be configured to ensure that the fluid composition is sealed within the fluid reservoir (610) when the connector is removed, and that the fluid composition does not proximally exit the fluid reservoir (610) through the plug lumen (618). The valve may generally ensure that fluid flow occurs from the proximal end of the handle (602) (e.g., when the fluid composition is loaded into the fluid reservoir (610)) to the distal end of the handle (602) (e.g., when the fluid composition is delivered through the elongate member to Schlemm's canal), and not vice versa. In some variations, the valve may include a one-way valve. In some instances, the valve may be a ball valve, a check valve including a ball check valve, single-piece duckbill or similar simple valve, or the like. In some variations, the valve may be constructed of a single silicone component.

In some variations, a wall of the proximal cavity (634) may include one or more abutments (654A/654B) extending into the proximal cavity (634) configured to allow the connector to be secured to the handle. The one or more abutments (654A/654B) may be located at a proximal end of the threaded engagement (632). When the connector is engaged with the coupling hub (614), the one or more abutments (654A/654B) may prevent further rotational movement of the connector in a first direction. Furthermore, as the connector (620) is disengaged from the coupling hub (614) in a second direction opposite the first direction, the connector (620) may contact the one or more abutments (654A/654B), preventing further rotational movement in the second direction and indicating to the user the connector (620) has fully disengaged the coupling hub (614) and may be removed from the proximal opening (612) in the proximal direction.

### Cannula

Referring back to FIG. 2, the cannula (208) of the delivery system (200) may generally be coupled to and extend from the distal end of the housing (206) of the handle (202). The cannula (208) may be configured to provide easy and minimally traumatic access to Schlemm's canal, such as during a minimally invasive ab-interno procedure. In some variations, the cannula (208) may be fixedly attached to the distal end of the housing (206). In other variations, the cannula (208) may be rotatably attached to the distal end of the housing (206) (e.g., via a rotatable hub or the like) to change the orientation of the tip of the cannula (208). In variations of the delivery systems in which the handle (202) is reusable and the cannula (208) is disposable, the cannula (208) may be removably attached to the distal end of the housing (206).

Some variations of the cannula (208) may include multiple portions having different geometric configurations. For example, in some variations, the cannula (208) may comprise a proximal end, a straight portion and a curved portion distal to the straight portion, where the curved portion has a proximal end and a distal end, and a radius of curvature. In other variations, generally, the cannula may comprise a curved portion, where the curve portion may comprise a first curved portion and a second curved portion. The second curved portion may comprise a radius of curvature greater than the first curved portion. However, it should be appreciated that in other variations, the cannula may be entirely straight (e.g., may comprise only a straight portion and may not comprise a curved portion). The cannula may also comprise a distal tip and a lumen extending from the proximal end through the distal tip. The lumen of the cannula may be in fluid communication with components of a fluid assembly as described above. In some variations, the distal tip may comprise one or more angled surfaces and may further include a sharpened piercing tip as is described in more detail below.

The cannula may be made from any suitable material with sufficient stiffness and biocompatibility to allow it to be advanced through the anterior chamber and into Schlemm's canal. For example, the cannula may be formed of a metal such as stainless steel, titanium, aluminum, or alloys thereof (e.g., Nitinol metal alloy), a polymer, ceramic, or a composite. Exemplary polymers include without limitation, polycarbonate, polyetheretherketone (PEEK), PEKK, PEI, polyimide, polyamide, polysulfone, or fluoropolymers. In some instances, it may be advantageous to coat the cannula with a lubricious polymer to reduce friction between the ocular tissue and the cannula during the procedure. Lubricious polymers include, without limitation, hydrophilic coatings (such as polysaccharides), hydrophobic coatings, polyvinyl alcohol, polyethylene glycol, polyvinyl pyrrolidone, fluorinated polymers (including polytetrafluoroethylene (PTFE or Teflon®)), and polyethylene oxide. In variations in which the cannula is reusable, the cannula may be made from a material that can be sterilized (e.g., via autoclaving), such as a heat-resistant metal (e.g., stainless steel, aluminum, titanium).

The cannula may generally have an outer diameter sized to gain access to the lumen of Schlemm's canal while minimally obstructing the surgeon's view. Accordingly, the outer diameter may range from about 50 microns to about 1000 microns. In some variations, the outer diameter may range from about 150 microns to about 800 microns, from about 200 microns to about 700 microns, from about 300 microns to about 600 microns, or from about 400 microns to 500 microns. The cannula also has an inner diameter, which may range from about 50 microns to about 400 microns, from about 100 microns to about 350 microns, or from about 150 microns to about 300 microns. The cannula may also be formed to have any suitable cross-sectional shape, e.g., circular, elliptical, triangular, square, rectangular, or the like. In some variations, the cannula may comprise a tapered profile along its length.

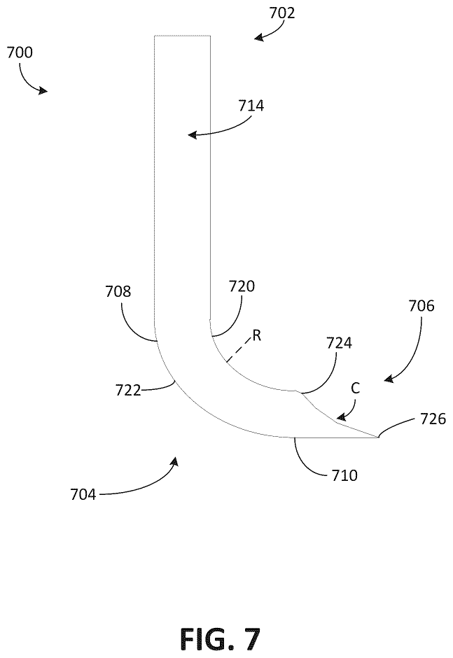

The cannula of an exemplary delivery system is shown in more detail in FIG. 7. As shown there, the cannula (700) may comprise a proximal end (702), a curved portion (704), a straight portion (714), and a distal tip (706). The straight portion may be proximal of the curved portion or the curved portion may be proximal of the straight portion. In some variations, the curved portion (704) may have two or more different radii of curvature (R). The curved portion (704) may have a proximal section (708) having a first radius of curvature and a distal section (710) having a second radius of curvature. In some variations, the radius of curvature (R) of the cannula may comprise two or more different radii of curvature including a compound curve or a reverse curve. The compound curve may have a first curve portion having a first radius of curvature bending in a first direction, and a second curve portion bending in the same direction as the first direction, having a second radius of curvature different from the first radius of curvature. The reserve curve may have a first curve portion having the first radius of curvature bending in the first direction and a second curve portion bending in a different direction from the first direction, having a second radius of curvature different from the first radius of curvature, as will be described in more detail herein.

The curved portion (704) may also have an inner radius (720) defined by the surface of the cannula (700) closest to the center of the radius of curvature (R), and an outer radius (722) defined by the surface of cannula further away from the center. The configuration of the curved portion (704) and the distal tip (706) may be beneficial or advantageous for allowing easy, atraumatic, and controlled access into Schlemm's canal. For example, the radius of curvature (R) may center the distal tip (706) along a longitudinal axis defined by a portion of the body (714) of the cannula (700), allowing movement of the proximal end (702) to easily translate to movement of the distal tip (706) to give a user more control of the distal tip (706). The distal tip (706) may have a proximal edge (724), a distal edge (726), and one or more radii of curvature (C) therebetween.

Utilizing a cannula (700) with a straight portion (714), a curved portion (704) having the proximal end (708) and the distal end (710), with a radius of curvature therebetween, and a distal tip (706) may be particularly useful for accessing the lumen of Schlemm's canal. A cannula (700) including a straight portion (714) and a curved portion (704) may have a length ranging from about 5 mm to about 50 mm, about 10 mm to about 30 mm, or from about 14 mm to about 20 mm. In some variations, the cannula (700) may have a length of about 18 mm. The curved portion of the cannula may be uniform in cross-sectional shape, or the shape may vary. In some variations, the cross-sectional size of the cannula may vary (e.g., the cannula may taper) along the curved portion, and in some instances, the cross-sectional size may be smaller closer to the distal end to facilitate entry into Schlemm's canal. The radius of curvature of the curved portion may be adapted to facilitate tangential entry, as well as precise and minimally traumatic entry into Schlemm's canal, and may range from about 1 mm to about 10 mm or from about 2 mm to about 5 mm. In one variation, the radius of curvature may be about 2.5. The cannula may also have a first angular span defined by the arc created by the inner radius (720) and the proximal edge (724). In some variations, the cannula may have a second angular span defined by the arc created by the outer radius (722) and the distal edge (726). The first or second angular spans may be suitable for facilitating entry into Schlemm's canal and may range from about 70 degrees to about 170 degrees, or about 100 degrees to about 150 degrees. In one embodiment, the first or second angular span may be about 100 degrees to about 120 degrees, including about 110 degrees.

The distal tip of the cannula may be sized, shaped, and have appropriate geometry to allow for easy and minimally traumatic access to Schlemm's canal. For example, the distal tip (706) may comprise a compound curve between the proximal edge (724) and the distal edge (726) that may facilitate sliding of the trabecular meshwork over the distal edge (726) and up the compound curve to provide an access point in the trabecular meshwork through which the elongate member may access Schlemm's canal, as will be described in more detail herein. It can be appreciated that the access point may include an opening to or within Schlemm's canal, including an opening in the trabecular meshwork.

In other variations, the cannula may include a straight portion positioned between the distal tip and the curved portion of the cannula. The length of the straight portion may range from about 0.5 mm to about 5 mm. In some variations, the length of the straight portion ranges from about 0.5 mm to about 3 mm, or from about 0.5 mm to about 1 mm. The length of the straight portion may also be a non-zero value less than about 0.5 mm, e.g., it may be about 0.1 mm, about 0.2 mm, about 0.3 mm, or about 0.4 mm. In some variations in which the distal tip is positioned directly adjacent (after) the curved portion of the cannula (i.e., the distal tip directly engages the radius of curvature), the cannula may lack a straight portion (length of the straight portion is zero).

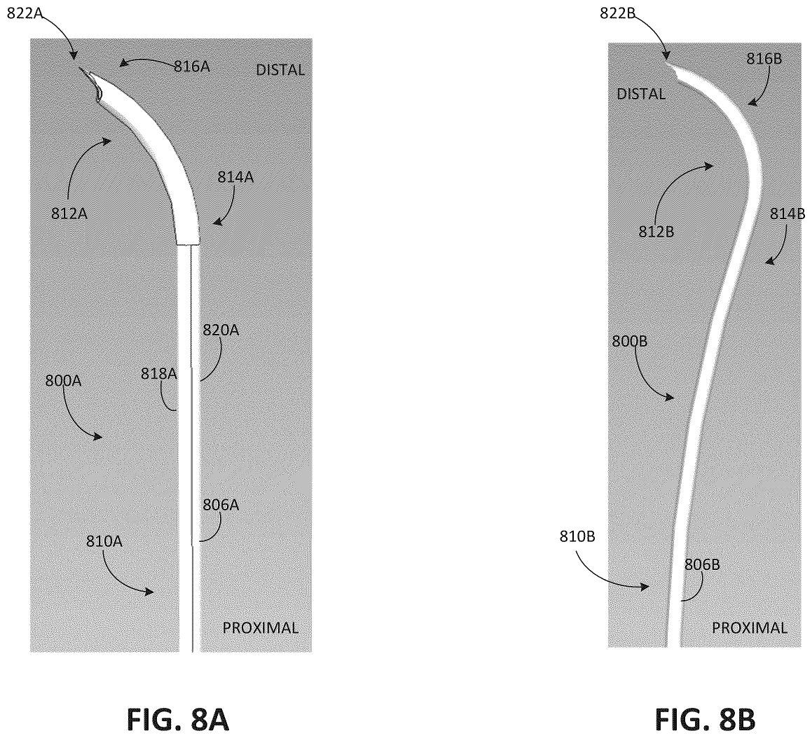

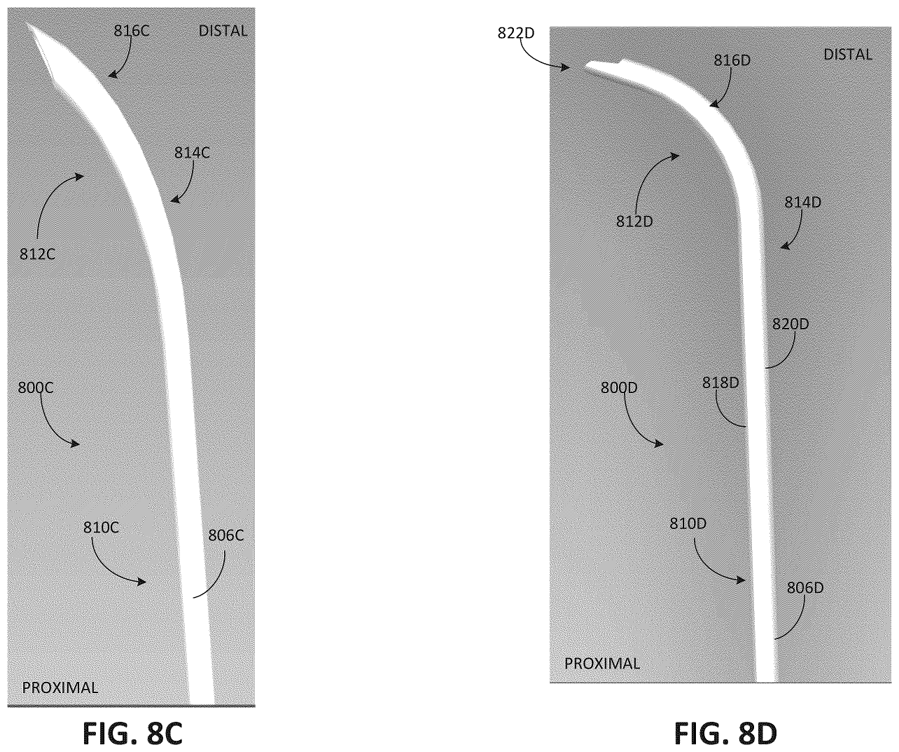

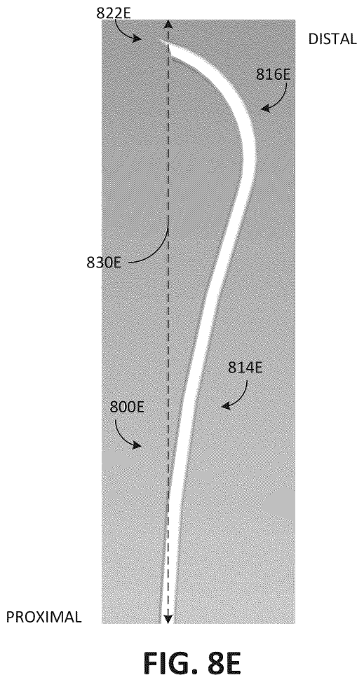

As generally described above, the cannula may comprise a proximal end, a curved portion, a straight portion, and a distal tip. The cannula may have an inner radius defined by the surface of the cannula closest to the radius of curvature and an outer radius defined by the surface of the cannula further away from the radius of curvature. The curved portion may terminate in the distal tip at either the outer radius or the inner radius. Different variations of the cannula may comprise different curved portion configurations. In some variations, the curved portion of the cannula may be configured to orient the trajectory of the elongate member as the elongate member extends from or advances out of the cannula. FIGS. 8A-8E depict profile views of variations of cannulas having different proximal ends and curved portions. Advantageously, the different variations of the cannula may allow a user more control of the distal tip of the cannula, resulting in ease of use in placing the cannula and seating the distal tip of the cannula within Schlemm's canal when delivering the fluid composition to the eye. For example, the different variations of the cannula described herein may allow the user to use a same access point location when treating both hemispheres of Schlemm's canal.

As depicted in FIG. 8A, the cannula (800A) may include a proximal end (810A), a straight portion (806A), and a curved portion (812A) having one or more radii of curvature. The proximal end (810A) may be substantially straight or may comprise a curve. The curved portion (812A) may have a proximal section (814A) and a distal section (816A) with a compound curve therebetween. In some variations, the proximal section (814A) and the distal section (816A) may be included in the compound curve or each of the proximal section (814A) and the distal section (816A) may be substantially straight with the compound curve therebetween. The compound curve may include two or more curves bending in the same direction with each of the two or more curves comprising a different radius of curvature. The proximal section (814A) of the curved portion (812A) may have a first radius of curvature and the distal section (816A) of the curved portion (812A) may have a second radius of curvature. In some variations, the second radius of curvature may be smaller than the first radius of curvature. Put differently, the proximal portion (814A) of the curved portion (812A) may have a flatter curve and the distal section (816A) of the curved portion (812A) may have a sharper curve. In some variations, the distal section (816A) of the curved portion (812A) may orient the trajectory of the elongate member as the elongate member extends from the distal section (816A) of the curved portion (812A) of the cannula (800A).

As illustrated in FIG. 8B, in some variations, the cannula (800B) may comprise a curved portion between the proximal end (810B) and the distal tip (822B), where the curved portion includes a reverse curve (e.g., a modified S-curve). The reverse curve may include two or more curves where at least one curve bends in a first direction having a first radius of curvature and at least one curve bends in a second direction opposite the first direction and comprises a second radius of curvature different from the first radius of curvature.

As described above, the compound curve between the proximal section (814) and the distal section (816) may have two or more different radii of curvature. In a variation illustrated in FIG. 8C, the distal section (816C) of the curved portion (812C) may comprise a second radius of curvature larger than the first radius of curvature of the proximal section (812C), leading to the proximal portion (814C) having a sharper curve and the distal portion (816C) having a flatter curve as compared to the embodiment depicted in FIG. 8A. In some variations, the distal section (816C) of the curved portion (812C) having a second radius of curvature larger than the first radius of curvature of the proximal section (814C) may orient the trajectory of the elongate member along the trajectory of the second radius of curvature of the distal section (816C) as the elongate member extends from the distal section (816C) of the curved portion (812C) of the cannula.

For example, in some variations, a compound curve may include a proximal section having a radius of curvature larger than a distal section, or the distal section may have a larger radius of curvature than the proximal section. In some variations, a ratio of the radius of curvature of the proximal section of the compound curve to the radius of curvature of the distal section of the compound curve may be about 10:1, about 9:1, about 8:1, about 7:1, about 6:1, about 5:1, about 4:1, about 3:1, or about 2:1. In some variations, a ratio of the radius of curvature of the distal section of the compound curve to the radius of curvature of the proximal section of the compound curve may be about 1:2, about 1:3, about 1:4, about 1:5, about 1:6, about 1:7, about 1:8, about 1:9, or about 1:10. For example, in a variation, the distal section may comprise a radius of curvature of about 0.10 inches and the proximal section may comprise a radius of curvature of about 1.00 inches. In some variations, the radius of curvature of the distal section may be about 0.05 inches to about 0.5 inches, and the radius of curvature of the proximal section may be around 0.5 inches to about 1.2 inches.

In each of these variations, the distal tip may be positioned on either the outer radius or the inner radius. FIG. 8D depicts a variation of a cannula (800D) having a curved portion (812D) with a proximal portion (814D) comprising a flatter curve and a distal section (816D) comprising a sharper curve. In this variation, the distal tip (822D) may be located on the inner radius (818D). Having the distal tip (822D) on the inner radius (818D) changes the orientation of the distal tip (822D) in relation to the user, and may provide additional flexibility for the user when placing the distal tip (822D) within Schlemm's canal.

As shown in FIG. 8E, the cannula (800E) may be curved and may comprise a proximal section (814E), a distal section (816E), which may comprise a compound curve. The proximal section (814E) may be curved and may comprise a first radius of curvature and the distal section (816E) may also be curved and may comprise a second, different radius of curvature. In this variation, the second radius of curvature may be greater than the first radius of curvature, and the first radius of curvature may be in an opposing direction as compared to the second radius of curvature. The combination of the first radius of curvature and the second radius of curvature being in opposing directions and having differing values may bias the distal tip (822E) of the cannula (800E) such that it is positioned along a central axis (830E) of the proximal section of the cannula (e.g., in variations in which the proximal section or a portion thereof is straight), but the majority of the cannula (800E) may be offset from the central axis. In some variations, the central axis of the cannula (830E) may bisect the distal tip (822E) of the cannula. Advantageously, configuring the cannula (800) such that the distal tip (822E) of the cannula (800E) is positioned along the central axis (830E) of the proximal section of the cannula (e.g., in variations in which the proximal section or a portion thereof is straight) may improve positioning of the distal tip (822E) relative to the handle such that it may be easier for a user to predict and understand the position of the distal tip (822E) relative to the anatomy of eye during a procedure, even when the distal tip may not be readily visually accessibly. In other variations including in which the proximal section of the cannula or a portion thereof is not straight, the distal tip (822E) of the cannula may be positioned along and/or bisect a central longitudinal axis of the handle. In some variations, the central longitudinal axis of the handle may be parallel to and/or substantially aligned with the central axis (830E) of the proximal section of the cannula. In some variations, the distal section (816E) comprising a second radius of curvature greater than the first radius of curvature may orient the trajectory of the elongate member as the elongate member extends from the cannula.

### Cannula Distal Tip

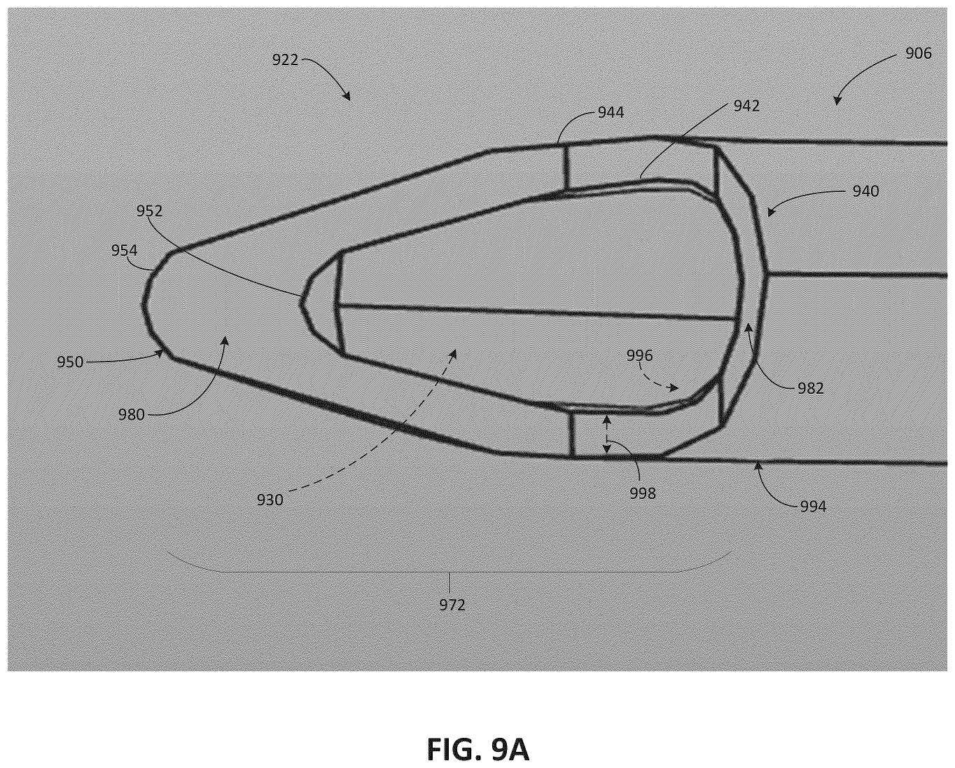

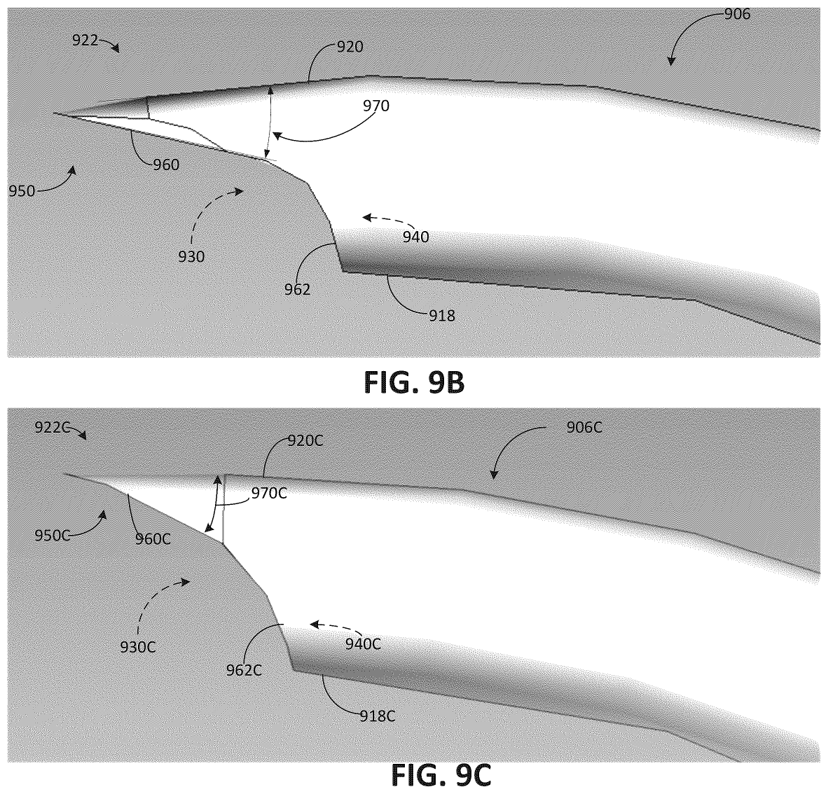

The distal tip of the cannula may be placed within Schlemm's canal and may be used to help form an access point within the trabecular meshwork for entry of the elongate member into Schlemm's canal. When deployed to Schlemm's canal, the distal tip may help form the access point within the trabecular meshwork. The configuration of the distal tip may allow a user to form and use the same access point (e.g., otomy) when delivering the fluid composition to both hemispheres of Schlemm's canal. The distal tip may be shaped into the various variations described herein, and the configuration of the distal tip may facilitate entry into and seating within Schlemm's canal. The configurations of the distal tip disclosed herein may be configured to easily bias or support the distal tip against the scleral wall without puncturing or piercing the scleral wall. Shaping the distal tip may include using standard manufacturing procedures including cutting, grinding, EDM, 3D printing, molding, or the like. The distal tip may be shaped by forming a pattern into the distal end of the cannula. Each embodiment described below may include beneficial features that provide an advantage for easy and minimally traumatic access to Schlemm's canal, for disrupting the trabecular meshwork and/or seating the distal tip within Schlemm's canal. FIG. 9A depicts a perspective view of the distal tip (922) of the cannula (906). Generally, the distal tip (922) may include a distal edge (950), a proximal edge (940), and a lumen opening (930) of the cannula (906). The elongate member may exit the cannula through the lumen opening (930). The lumen opening (930) may comprise any suitable shape, including but not limited to, a tear drop, an oval, a circle, a pentagon, a hexagon, an irregular shape, or the like.

The proximal edge (940) may be straight or rounded. In some variations, the proximal edge (940) may comprise an inner proximal edge (942) and an outer proximal edge (944). The inner proximal edge (942) and the outer proximal edge (944) may define a base (982). The distal edge (950) may comprise an inner distal edge (952) and an outer distal edge (954). The inner distal edge (952) and the outer distal edge (950) may define a tongue (980). The tongue (980) may be used to disrupt the trabecular meshwork and may be seated within Schlemm's canal during delivery of the fluid composition to the eye. Generally, the distal tip may have a length. For example, the distal tip (922) may have a length (972) from a proximal edge (940) to a distal edge (950). Generally, the tongue may have a length, where generally, the length of the tongue may include the distance along a longitudinal axis from the inner distal edge to the outer distal edge. The length of the tongue may comprise a percentage of the length of the distal tip. For example, the length of the tongue may comprise about 1% to about 50% the length of the distal tip, including about 1% to about 5%, or about 5% to about 10%, or about 10% to about 15%, or about 15% to about 25% (including all values and sub-ranges therein). In some variations, the distal tip may have a diameter that tapers from the proximal edge to the distal edge, or the diameter of the distal tip may be constant from the proximal edge to the distal edge.

In some variations, the distal edge (950) may be rounded or straight. The distal edge (950) may be sharp and used to move and/or penetrate the trabecular meshwork. In some variations, one or more, including all, surfaces of the distal tip (922) including the distal edge (950), the straight portion (960), the proximal edge (940), the curved portion (962), the base (982), and the tongue (980) may be substantially smooth (e.g., electropolished) to prevent damage to the elongate member. Generally, the cannula having the lumen therein, may have a cannula wall having a wall thickness. For example, as illustrated in FIG. 9A, the cannula (906) may comprise a wall thickness (998) defined by an outer wall (994) of the cannula (906) and an inner wall (994) of the cannula (906). The wall thickness may be constant along a longitudinal axis of the cannula (e.g., from the proximal end to the distal tip) or the wall thickness may be varied. For example, in some variations, the wall thickness (998) may be constant from the proximal end to the distal tip (922), or the wall thickness (998) may be constant along the length (972) of the distal tip (922). In other variations, the wall thickness (998) may be varied, including at specific locations along the cannula. For example, the wall thickness (998) along the distal tip (922) may be tapered along the length (972), wherein the wall thickness (998) at the proximal edge (940) may be substantially greater (e.g., 2x, 3x, 4x or the like) than the wall thickness (998) at the distal edge (950). In some variations, the tapering of the wall thickness along the distal tip may allow the distal tip to more easily penetrate the trabecular meshwork, creating an access point therein through which to deploy the elongate member. In some variations, the distal tip may include side cuts, configured to narrow a diameter of the tongue, allowing the tongue to seat into Schlemm's canal.

FIG. 9B depicts a side perspective view of an embodiment of the distal tip (922) of FIG. 9A. As described above, the cannula (906) may include an inner radius (918) defined by the surface of the cannula (906) closest to the radius of curvature, and an outer radius (920) defined by the surface of the cannula (906) further away from the radius of curvature. Furthermore, the distal tip (922) includes the distal edge (950) comprising a straight portion (960) of the distal tip followed by the proximal edge (940) comprising a curved portion (962) of the distal tip. In some variations, the straight portion (960) may be chamfered or may not be chamfered. The straight portion (960) of the distal tip may be distal the curved portion (962) of the distal tip. The straight portion (960) and the outer radius (920) may form an angle (970), as illustrated in FIG. 9B. The angle (970) may generally be acute, and may, in some variations, be within the range of about 14 degrees to about 40 degrees (including all values and sub-ranges therein). For example, in some variations, the angle (970) may be about 14 degrees to about 20 degrees, about 22.5 degrees to about 28.5 degrees, about 18 degrees to about 26 degrees, or about 30 degrees to about 40 degrees. Utilizing an angle (970) from about 14 degrees to about 40 degrees may advantageously allow the distal tip (922) to be placed tangent to Schlemm's canal when inserted therein, thus allowing the elongate member to easily follow the trajectory of the cannula when extended therefrom, for proper placement of the elongate member in Schlemm's canal.

In some variation, the straight portion (960) of the distal tip may have one or more straight segments (e.g., two, three, four or more). In variations in which the straight portion (960) of the distal tip has a plurality of segments, each segment may be substantially straight, but the segments may have different slopes. Similarly, in variations in which the curved portion (962) of the distal tip has a plurality of segments, each segment may have a different radius of curvature. Put differently, in variation in which the curved portion (962) of the distal tip may have a plurality of segments, the curved portion may comprise a compound curve. The straight portion (960) of the distal tip and the curved portion (962) of the distal tip may each have any appropriate number of segments, and it should be appreciated that any combination of straight portions (960) of the distal tip and curved portions (962) of the distal tip may be utilized. For example, in some variations, a straight portion of the distal tip with a single straight segment may be utilized with a curved portion of the distal tip comprising a plurality of curves with different radii of curvature, while in other variations, a straight portion of the distal tip with a plurality of straight segments with differing slopes may be used with a curved portion with a distal tip with a single curve (the entire curved portion may have a single radius of curvature). Similarly, a straight portion of the distal tip with a plurality of straight segments with differing slopes may be utilized with a curved portion of the distal tip with a plurality of curved segments.

FIG. 9C depicts a variation of a distal tip (922C) of the cannula (906C) similar to that depicted in FIGS. 9A-9B. In this variation, the distal tip (922C) comprises a straight portion (960C) of the distal tip and a curved portion (962C) of the distal tip. The straight portion (960C) of the distal tip and the outer radius (920C) may form an angle (970C) within the range of about 18 degrees to about 26 degrees. With the angle (970C) being within the range of about 18 degrees to about 26 degrees, the curved portion (962C) of the distal tip may have a shallower curved compared to the curved portion (962) of the distal tip in FIG. 9B. Furthermore, this variation allows trabecular meshwork to slide up the straight portion (960C) of the distal tip and at least partially up the curved portion (962C) of the distal tip, forming an access point for the elongate member to exit the lumen opening (930C) of the cannula (906) and enter Schlemm's canal.

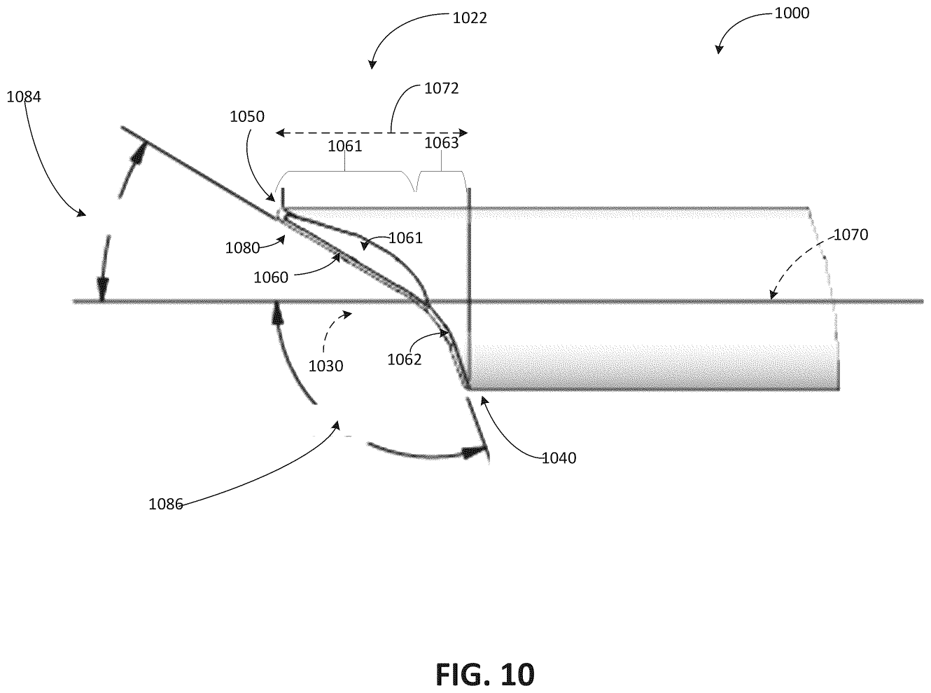

In some variations, the distal tip may include a longitudinal midpoint configured to partition the cannula (1000) into two sections. FIG. 10 shows another variation of a distal tip (1022). In this variation, the distal tip (1022) may comprise a longitudinal midpoint (1070) and a straight portion (1060) of the distal tip that may form a first angle (1084). The distal tip (1022) may further comprise a second angle (1086) formed between the longitudinal midpoint (1070) and a proximal edge (1040). The first angle (1084) may generally be within the range of about 14 degrees to about 35 degrees (including all values and sub-ranges therein). For example, the first angle (1084) may be within the range of about 27 degrees to about 35 degrees or about 14 degrees to about 22 degrees. The second angle (1086) may generally be obtuse and within the range of about 90 degrees to about 160 degrees (including all values and sub-ranges therein). For example, in some variations, the second angle (1086) may be within the range of about 100 degrees to about 120 degrees.

The distal tip described herein may generally have a length measured between the distal edge and the proximal edge along the longitudinal axis of the device. For example, FIG. 10 depicts a variation of a distal tip (1022) comprising a length (1072) measured from the proximal edge (1040) to the distal edge (1050) along the longitudinal axis (1070). The length of the distal tips described herein may, in some variations be about 0.013 mm to about 0.035 mm (including all values and sub-ranges therein). For example, the length may be from about 0.0145 mm to about 0.0185 mm, or about 0.015 mm to about 0.019 mm, or about 0.02 mm to about 0.03 mm. In some variations, one or more of, including all, of the distal edge, the proximal edge, the straight portion, and the curved portion of the distal tips described herein may have chamfered edges, smooth edges, or a combination thereof. In variations comprising both a straight portion and a curved portion, the length may include corresponding lengths of the straight portion and/or the curved portion, each similarly measured along the longitudinal axis. This can be seen in FIG. 10, where length (1072) is the length of the distal tip, length (1061) is the length of the straight portion (1060) and length (1063) is the length of the curved portion (1062). In some variations, the straight portion and the curved portion may comprise an equal percentage of the length of the distal tip or the length of the straight portion may comprise a greater percentage of the length of the distal tip than the length of the curved portion. For example, in some variations in which the length of the straight portion comprises a greater percentage than the length of the curved portion, the length of the straight portion may be 51%, 55%, 60%, 65%, 70%, 75%, 80%, 85% or 90% of the length of the distal tip. As another example, in some variations in which the length of the curved portion may comprise a greater percentage than the length of the straight portion, the length of the curved portion may be 51%, 55%, 60%, 65%, 70%, 75%, 80%, 85% or 90% of the length of the distal tip. In some variations, the ratio of the length of straight portion to the length of the curved portion may be about 1:1, 1.5:1, 1.75:1, 2:1, 2.5:1, 3:1, 1:1.5, 1:1.75, 1:2, 1:2.5, or 1:3. In variations comprising both a straight portion and a curved portion, the straight portion may have a length within the range of about 0.066 mm to about 0.032 mm and the curved portion may have a length within the range of about 0.003 mm to about 0.063 mm. In other variations comprising both the straight portion and the curved portion, the curved portion may have a length within the range of about 0.066 mm to about 0.032 mm and the straight portion may have a length within the range of about 0.003 mm to about 0.063 mm. As described about, generally, the length of the straight portion may include the length of the tongue. In other words, the length of the tongue is a subset of the length of the straight portion. In some variations, the length (1072) may comprise the length (1061) of the straight portion (1060) including the length of the tongue (1080) and the length (1063) of the curved portion (1062). In some variations, the length of the tongue may be within the range of about 0.001 mm to about 0.02 mm. In some variations, the straight portion (1060) may comprise side cuts (1061), configured to allow the straight portion (1060) to be inserted into Schlemm's canal.

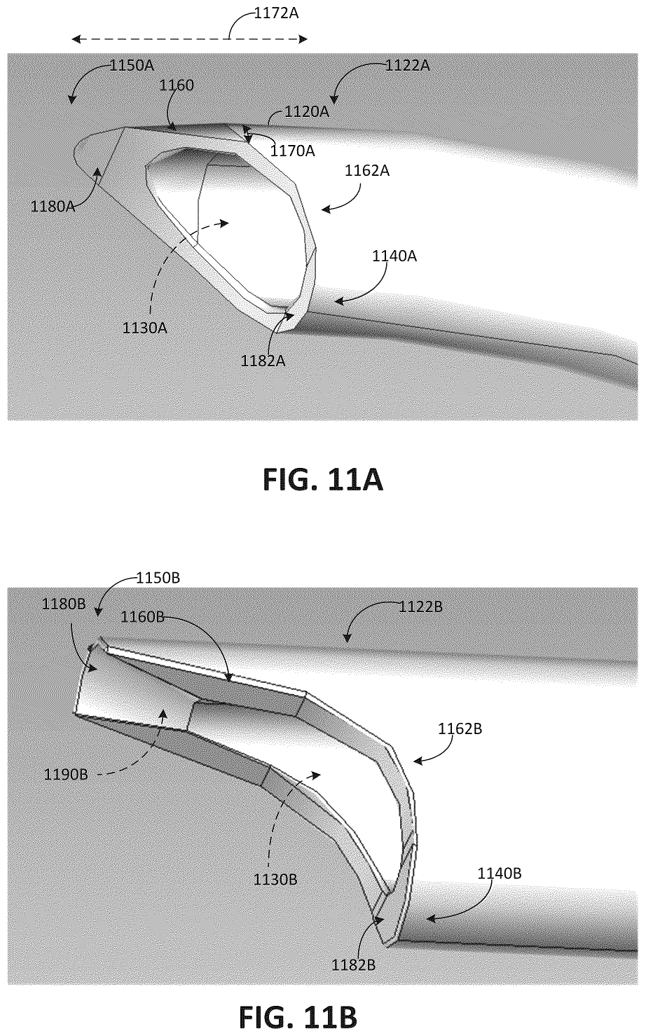

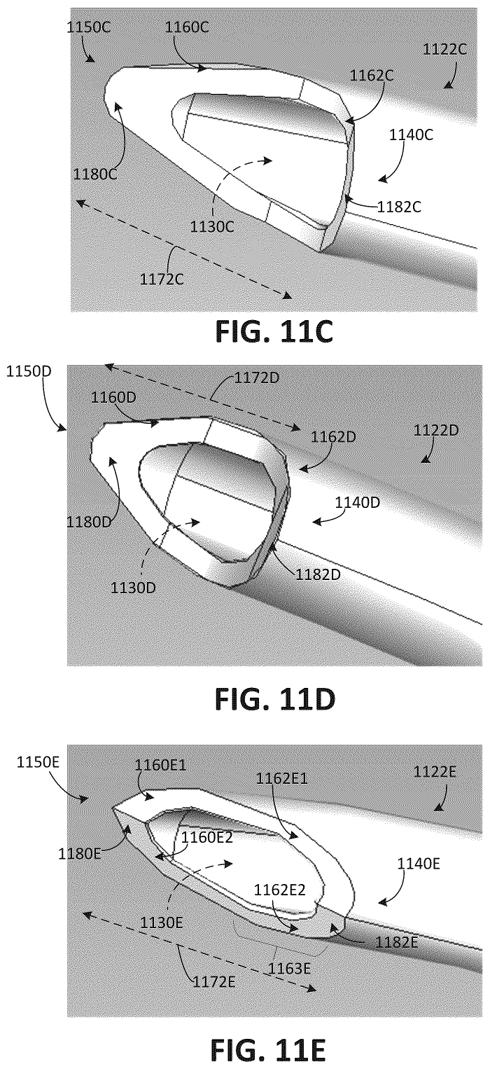

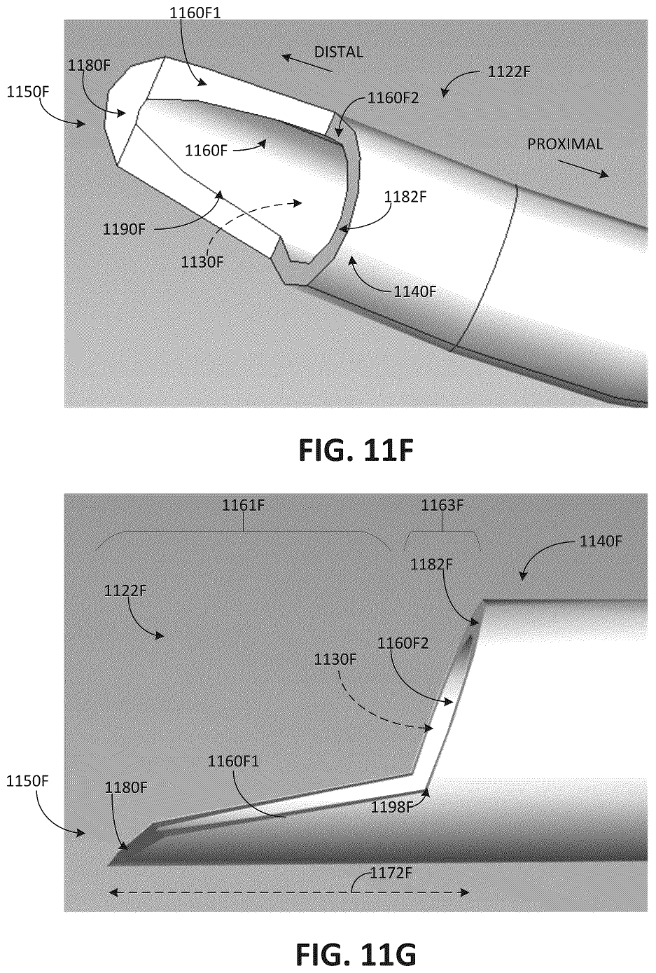

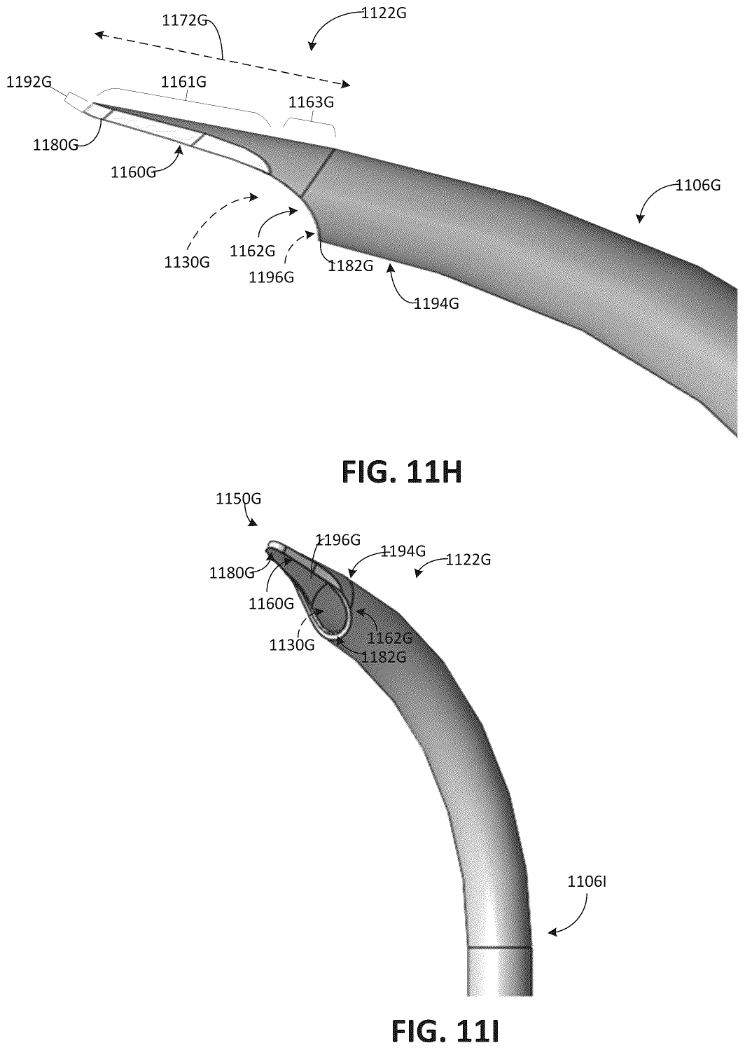

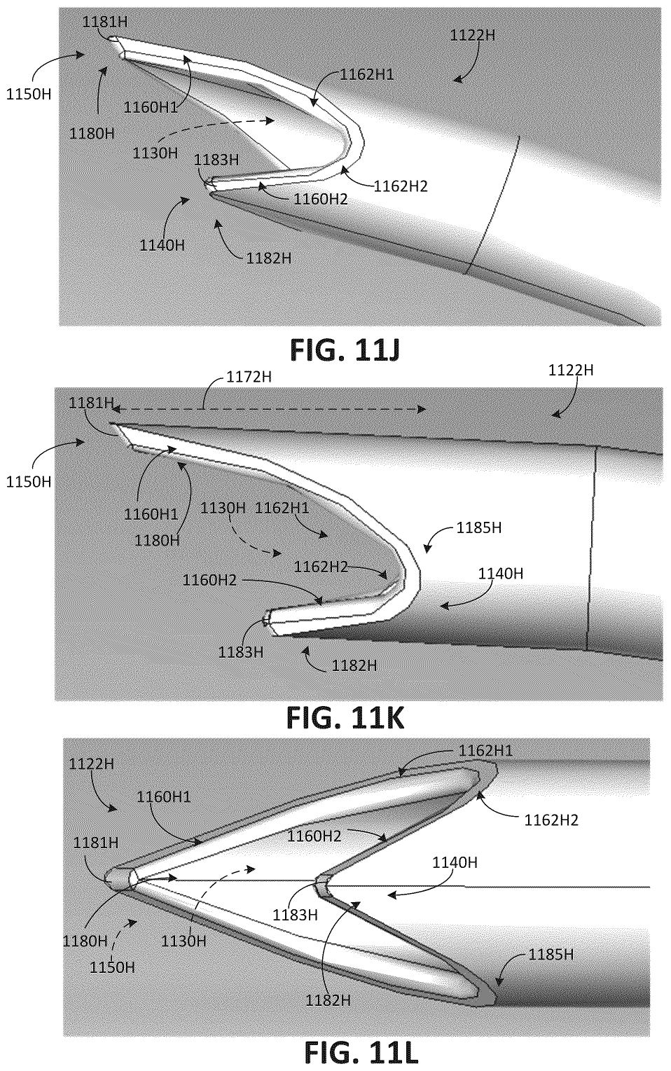

FIGS. 11A-11L illustrate views of different variations of the distal tip (1122). Each of the variations of the distal tip (1122A-1122L) may be combined with any disclosed embodiment of the cannula (as described above and illustrated in FIGS. 8A-8E) to form a cannula that may be used in the delivery system. Referring now to FIG. 11A, shown there is the distal tip (1122A) having a length (1172A) that comprises the straight portion (1160A) of the distal tip and the curved portion (1162A). With a defined length (1172A), a longer straight portion (1160A) ensures the curved portion (1162A) comprises a sharper curve while a shorter straight portion (1160A) ensures the curved portion (1162A) may comprise a flatter curve. In some variations, when straight portion (1160A) comprises a greater percentage of the length (1172A) compared to the curved portion (1162A), the distal tip (1122A) may be inserted deeper into Schlemm's canal. Each of the different variations of the distal tip (1122A-1122L) illustrated in FIG. 11A-11L may be configured to allow the trabecular meshwork to slide up straight portion of the distal tip and a portion of the curved portion of the distal tip to provide an access point through the trabecular meshwork through which the elongate member may access Schlemm's canal.

As illustrated in FIG. 11A, the curved portion (1162A) may comprise a compound curve with two curves bending in the same direction, each curve having a different radius of curvature. The angle (1170A) is the angle between the straight portion (1160) and the outer radius (1120A), and in some variations, may be within the range of about 14 degrees to about 20 degrees. In some variations, the distal tip (1122A) may taper from the proximal edge (1140A) to the distal edge (1150A). The distal edge (1150A) may be rounded or curved to allow the distal tip (1122A) to be placed against the scleral wall or the anterior chamber without damaging or puncturing the scleral wall or anterior chamber during deployment.

In some variations, instead of tapering from the proximal edge to the distal edge, the distal tip may have a constant diameter. Additionally, or alternatively, in some variations, the distal edge (1150B) may be is straight instead of rounded, as illustrated in FIG. 11B. The straight distal edge (1150B) may easily pierce through the trabecular meshwork. In some variations, the tongue (1180B) may further include a tongue channel (1190B) extending from the lumen opening (1130B) to the distal edge (1150B). The tongue channel (1190B) may facilitate the sliding of the trabecular meshwork through the tongue (1180B) and up the curved portion (1162B). The tongue channel (1190B) may facilitate the sliding of the trabecular meshwork inward towards the lumen opening (1130B). The tongue channel (1190B) may also be configured to constrain and guide the elongate member out of the lumen opening (1130B).

In some variations, the straight portion may have an elongated taper along the length which may allow the tongue and the straight portion to sit deeper within Schlemm's canal during deployment of the distal tip. For example, as illustrated in FIG. 11C the straight portion (1160C) includes an elongated taper along the length (1172C), allowing the tongue (1180C) and the straight portion (1160C) to sit deeper within Schlemm's canal during deployment of the distal tip (1122C). The elongated taper of the straight portion (1160C) may provide a gradual lift of the trabecular meshwork during deployment of the distal tip (1122C). In some variations, the straight portion (1160C) may have chamfered, beveled or rounded edges.

As depicted in FIG. 11D, the straight portion (1160D) may have a sharper taper along the length (1172D) compared to the variation of the distal tip (1122C) depicted in FIG. 11C. The sharper taper of the straight portion (1160D) may provide an overall shorter length (1172D) of the distal tip (1122D), allowing the user to perform meticulous movements of the distal tip (1122D). Generally speaking, in some variations, the distal tip length may comprise equal portions of the length of the straight portion and the length of the curved portion. However, as described above, in some variations, one of the lengths of the straight portion (1160D) and the length of the curved portion (1162D) may comprise a greater percentage of the distal tip length (1172D) (e.g., >50%). For example, in FIG. 11C the length of the straight portion (1160C) comprises a greater percentage of the distal tip length (1172C), while in FIG. 11D, the length of the curved portion (1162D) comprises a greater percentage of the distal tip length (1172D). Furthermore, in FIG. 11A, the length of the straight portion (1160A) and the length of the curved portion (1162A) comprise equal percentages of the distal tip length (1172A).

The lumen opening formed by and within the distal tip and may have a variety of suitable shapes and sizes. For example, the lumen opening may be circular, semi-circular, ovular, semi-ovular, or the like and may have a diameter or major axis about 0.005 mm to about 0.02 mm. For example, FIG. 11E depicts a variation of the distal tip (1122E) having an oval-shaped lumen opening (1130E). In some variations, the distal tip (1122E) may comprise a first straight portion (1160E1), a second straight portion (1160E2), a first curved portion (1162E1) and a second curved portion (1162E2). In shaping the distal tip (1122E), each of the first straight portion (1160E1) and first curved portion (1162E1) and the second straight portion (1160E2) and the second curved portion (1162E2) comprise shallow angles that meet along a midline of the distal tip (1122E). The first straight portion (1160E1) and the second straight portion (1160E2) may be joined at the distal edge (1150E), wherein the distal edge (1150E) may be a point or may be rounded. The first curved portion (1162E1) and the second curved portion (1162E2) may be joined at the proximal edge (1140E), wherein the proximal edge (1140E) may be a point or may be rounded. The distal tip (1122F) may be configured to easily penetrate the trabecular meshwork and lift the trabecular meshwork over the distal tip of the cannula.

As noted above, the straight portion of the distal tip may comprise a first straight portion and a second straight portion with the first straight portion being distal to the second straight portion. Referring now to FIGS. 11F and 11G, shown there is a distal tip (1122F) comprising a first straight portion (1160F1) and a second straight portion (1160F2). In this variation seen in FIG. 11G, the distal tip length (1172F) includes the length of the first straight portion (1161F) and the length of the second straight portion (1163F) only. Put differently, there is no curved portion in this variation. In this embodiment as seen in FIG. 11F, the distal tip (1122F) may include a channel (1190F). In some variations, as can be seen in FIG. 11F, the channel (1190F) may taper distally while in other variations, the channel (1190F) may taper proximally or may have a constant width such that is does not taper. The channel (1190F) may be configured to constrain and guide the elongate member along the direction of the channel (1190F), as the elongate member is actuated out of the lumen opening (1130F). The first straight portion (1160F1) and the second straight portion (1160F2) may form a first angle (1198F). In some variations, the first angle (1198F) may be about 90 degrees to about 150 degrees (including any values and sub-ranges therein). The first straight portion (1160F1) may be configured to lift the trabecular meshwork as the meshwork slides up the first straight portion (1160F1), allowing the elongate member to easily enter Schlemm's canal. The first straight portion (1160F1) may also be inserted into Schlemm's canal. The second straight portion (1160F2) may be configured to constrain the elongate member as the elongate member exits the cannula at the cannula lumen opening. The first straight portion (1160F1) may have a smaller (i.e., flatter) slope than the second straight portion (1160F2). The configuration of the first and second straight portion (1160F1, 1160F2) may assist in forming a sufficiently large access point within the trabecular meshwork, while constraining the elongate member as the elongate member moves through the access point into Schlemm's canal. In some variations, constraining the elongate member includes the elongate member maintaining the proper trajectory as the elongate member exits the cannula.

FIG. 11H illustrates another variation of the distal tip (1122G). In this variation, the distal tip (1122G) comprises a straight portion (1160G) having a chamfered edge and a curved portion (1162G), wherein the length (1162G) of the straight portion (1160G) may comprise a greater percentage of the distal tip length (1172G) than the length (1163G) of the curved portion (1162G), allowing the user to place the distal tip (1122G) further into Schlemm's canal. Furthermore, the cannula may comprise a cannula wall thickness defined by an outer wall of the cannula and an inner wall of the cannula. For example, as illustrated in FIG. 11I the cannula (1106G) may comprise a wall thickness (1192G) defined by the outer wall (1194G) and the inner wall (1196G). The wall thickness may vary along the distal tip length including tapering distally towards the distal edge. For example, the wall thickness may be within the range of about 0.01 mm to about 0.05 mm or within the range of about 0.0010 inches to about 0.0050 inches thick. The wall thickness may be about 0.01 mm to about 0.04 mm, about 0.01 to about 0.03 mm, or about 0.01 mm to about 0.02 mm. The wall thickness may be about 0.01 mm to about 0.04 mm, about 0.02 mm to about 0.03 mm or 0.02 mm to about 0.05 mm, about 0.03 mm to about 0.05 mm or about 0.04 mm to about 0.05 mm. The wall thickness may be about 0.0010 inches to about 0.0040 inches, about 0.0010 inches to about 0.0030 inches, or about 0.0010 inches to about 0.0020 inches. The wall thickness may be about 0.0020 inches to about 0.0050 inches, about 0.003 inches to about 0.0050 inches, or about 0.0040 inches to about 0.0050 inches. In some variations, the wall thickness may be uniform along the distal length, may taper uniformly along the distal length or may taper rapidly towards the distal edge. In some variations, the wall thickness (1192G) may stay constant along the distal tip length (1172G) as illustrated in FIG. 11H. The uniform wall thickness (1192G) along the distal tip length (1172G) may be configured so that the cross-sectional area of the tongue (1180G) fits within Schlemm's canal.

As described above the distal edge may be straight or rounded. For example, as illustrated in FIG. 11I, the distal edge (1150G) may have a rounded edge configured to be placed against the scleral wall. The constant wall thickness (1192G) and the rounded configuration of the distal edge (1150G) may allow the user to confidently place the distal edge (1150G) against the scleral wall without puncturing the scleral wall and minimizing the risk of injuring scleral wall tissue, which would require significant perceivable force.

In some variations, the base of the distal tip may extend distally to a pointed edge. In some of these variations, both the base and the tongue may extend to an apex. In some variations, one or more of each of the apex may be rounded, while in other variations, one or more of each of the apex may not be rounded (e.g., it may be angular). Referring now to FIGS. 11J-11L, the distal tip (1122H) may comprise a base (1182H) that extends to a base apex (1183H) and a tongue (1180H) that extends to a tongue apex (1181H). The base apex (1183H) may be offset proximally from the tongue apex (1181H), as is depicted in FIGS. 11J-11L. Put differently, in some variations, the tongue may extend distally behind the base, such that the base apex (1183H) is proximal of the tongue apex (1181H). In other variations, the base apex and the tongue apex maybe axially aligned, such that the base apex and the tongue apex are parallel to one another. In some variations, the base apex may have a larger radius of curvature compared to the tongue apex, leading to a rounder shape at the base apex, while in other variations, the tongue apex may have a larger radius of curvature compared to the base apex, leading to the rounder shape at the tongue apex. In other variations, the tongue apex and the base apex may have the same radius of curvature, wherein the tongue apex and the base apex have the same shape (see FIG. 11L).

Advantageously, in this variation, each of the tongue apex (1181H) and the base apex (1183H) may simultaneously encounter trabecular meshwork during deployment to the eye, leading to an easily formed access point to Schlemm's canal. In some variations, the distal edge (1150H) may have the first straight portion (1160H1) followed by the first curved portion (1162H1, where the first curved (1162H1) portion is positioned proximally of the first straight portion (1160H1) and includes a compound curve. In some variations, the proximal edge (1140H) may include straight and curved portions. For example, the proximal edge (1140H) may comprise a second straight portion (1160H2) and a second curved portion (1162H2), where the second straight portion (1160H2) is distal to the second curved portion (1162H2), and the second curved portion (1162H) has a compound curve. In some variations, the curvature of the second curved portion (1162H2) may be opposite the curvature of the first curved portion (1162H1). In this embodiment, the second curved portion (1162H2) together with the first curved portion (1162H1) may form one continuous curve between and connecting the first straight portion (1160H1) and the second straight portion (1160H2). The distal tip length (1172H) may be measured from the proximal end (1195H) of the base (1182H) to the tongue apex (1181H). In this variation, the straight portion (1160H1/1160H2) comprise a greater percentage of the distal tip length (1172H) than the curved portion (1162H1/1162H2).

In some variations, each of the first and second straight portions and the first and second curved portion may comprise chamfered or beveled edges, as illustrated in FIG. 11K. The user may easily place any one of the surfaces or edges against the scleral wall during deployment including base apex (1183H), the tongue apex (1181H), the distal edge (1150H) or the proximal edge (1140H) without damage to the tissue. Furthermore, the user may use the distal tip (1122H) in either the tongue apex (1182H) up orientation as illustrated in FIG. 11J, or the base apex (1182H) up orientation, providing enhanced versatility of the distal tip (1122H) during deployment.

### Methods of Use

In some variations, the methods of treating conditions of the eye may comprise dilating Schlemm's canal and/or tearing the trabecular meshwork using a single delivery system. In some instances, treating conditions of the eye may result in increased aqueous humor drainage, reduced resistance to aqueous outflow, and/or reduced intraocular pressure. Some methods described herein may dilate Schlemm's canal, dilate the collector channels, and/or break any septae that may obstruct circumferential flow through Schlemm's canal. Dilation of Schlemm's canal may disrupt obstructed inner walls of the canal, stretch the trabecular meshwork, and/or increase the trabecular meshwork's porosity. This may improve the natural aqueous outflow pathway. The dilation may be performed by delivery of a fluid composition (e.g., a viscoelastic fluid as described herein). Some methods described here may comprise performing a trabeculotomy to cut trabecular meshwork. Some methods described here may comprise implanting an ocular device within Schlemm's canal. In some instances, the systems described herein may be used in performing ab-interno trabeculotomy, ab-interno transluminal trabeculotomy, clear corneal trabeculotomy, clear corneal transluminal trabeculotomy, ab-interno canaloplasty, and/or clear corneal canaloplasty.

### Loading A Fluid Composition Into The Delivery Device

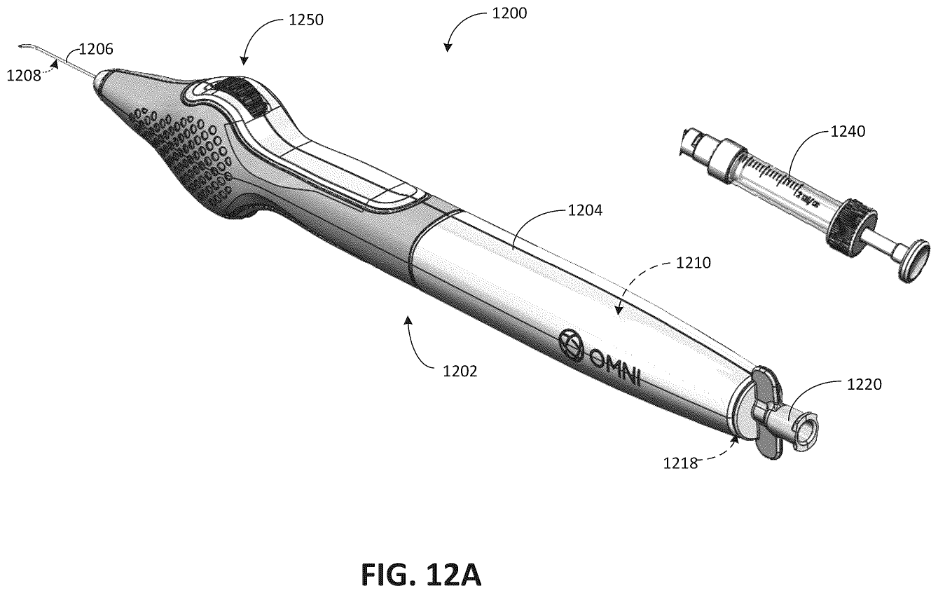

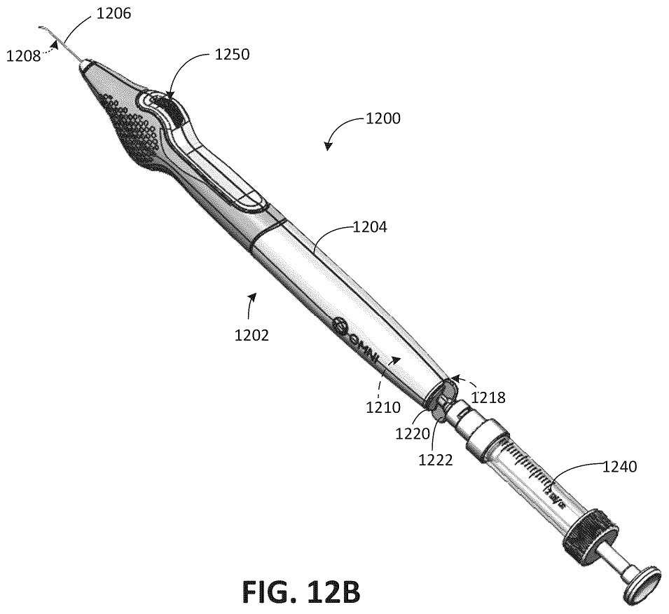

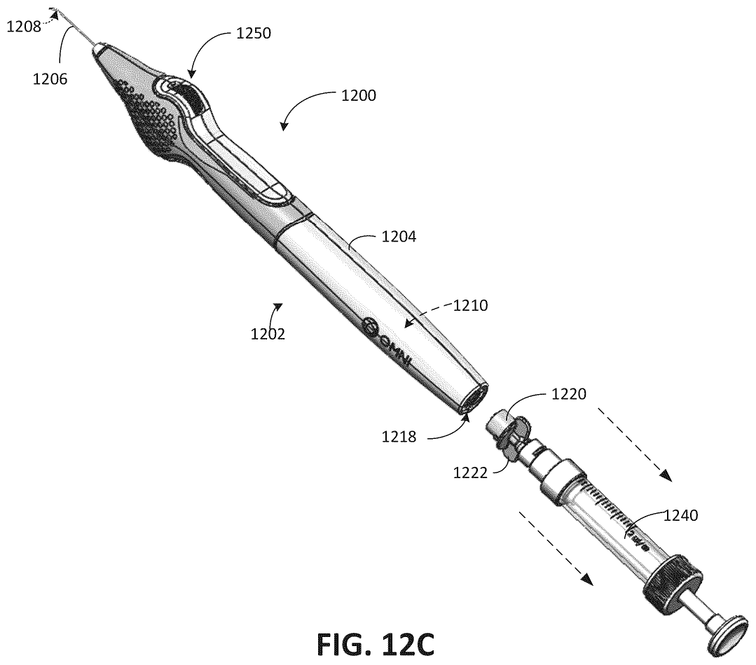

Before the fluid composition is delivered to the eye, the fluid composition may be loaded into a delivery device from an external fluid device. It may be important to maintain sterility of the fluid composition while quickly transferring the fluid composition from the external fluid device to a fluid reservoir within a housing of the delivery device without introducing any additional air into a fluid assembly and while removing any existing air within the fluid assembly. In some variations, delivery systems may include an external fluid device configured to be coupled to a connector, the connector removably coupled to the delivery device. The external fluid device may deliver fluid through the connector to a fluid reservoir within the delivery device. Having a connector removably coupled to the delivery device allows fluid to be seamlessly and in a sterile manner transferred from the external fluid device to the fluid reservoir. The fluid composition can quickly purge any existing air within the fluid assembly as fluid flows through the fluid assembly to a distal tip of an elongate member in fluid communication with the fluid reservoir. Once the fluid composition has been transferred, the connector with the external fluid device coupled thereto can be easily and quickly removed from the delivery device, allowing the delivery device to be deployed to the eye. FIGS. 12A-12C depict an exemplary method of delivering fluid from an external fluid device (1240) to the fluid reservoir (1210) of the delivery device (1200) including coupling an external fluid device to the handle of a delivery device using a connector of the fluid assembly of the delivery device, transferring the fluid composition into the fluid reservoir of the delivery device, and decoupling both the connector and the external fluid device from the handle of the delivery device.

As illustrated in FIG. 12A, the delivery device (1200) includes a handle (1202) having a housing (1204) with a cannula (1206) coupled to the distal end of the housing (1204). The delivery device (1200) may further include a connector (1220) coupled to a proximal opening (1218) of the housing (1204). In some variations, methods may include filling the external fluid device (1240) with a volume of fluid composition. The contents of the fluid composition may be as described above. The external fluid device (1240) may include a syringe, a vial, or another container used to store fluid.

In some variations, methods may further include detachably coupling the external fluid device (1240) to the connector (1220). Once the external fluid device (1240) is coupled to the connector (1220), methods may further include transferring the volume of the fluid composition from the external fluid device (1240) to the fluid reservoir (1210). Transferring the volume of the fluid composition from the external fluid device (1240) to the fluid reservoir (1210) may include transferring the volume of fluid through the connector (1220) to the fluid reservoir (1210). The transfer of the fluid composition from the external fluid device (1240) to the fluid reservoir (1220) may be active or passive. The fluid reservoir (1220) may be sized to receive a defined volume of the fluid composition from the external fluid device (1240). In some variations, a volume of the fluid composition greater than the defined volume of the fluid reservoir (1210) may be transferred to the delivery device (1200) with the additional volume of the fluid composition used to prime the delivery device (1200) by removing any air within the fluid assembly and/or the elongate member. The excess fluid may flow out of the distal tip of the cannula (1206) or the elongate member, indicating to the user that the delivery device (1200) is primed with fluid and is ready to deliver fluid to the eye.

Methods of delivering fluid from the external fluid device to the fluid reservoir may further include removing the external fluid device (1240) and the connector (1220) from the handle (1202), as illustrated in FIG. 12C. Removing the external fluid device (1240) and the connector (1220) may include removing the connector (1220) while it remains coupled to the external fluid device (1240). Put another way, removing the external fluid device (1240) and the connector (1220) may include removing the external fluid device (1240) and the connector (1220) together (e.g., simultaneously and while they remain coupled to one another). Removing the external fluid device (1240) and the connector (1220) from the handle (1202) may include engaging tabs (1222) of the connector (1220) to disengage the connector (1220) from the handle (1202). In some variations, engaging the tabs (1222) may include rotating the tabs (1222) in a first direction. Additionally, or alternatively, engaging the tabs (1222) may include pulling the tabs (122) axially away from the handle housing. Removing the external fluid device (1240) and the connector (1220) from the handle (1202) may include retracting or otherwise pulling the external fluid device (1240) and the connector (1220) proximally from the handle (1202). In some variations, removing the external fluid device and the connector from the handle may include pressing a release button on the handle and removing the connector from the handle.

The coupled connector (1220) and external fluid device (1240) may be removed from the delivery device (1200) as illustrated in FIG. 12C. Removal of the connector (1220) and the external fluid device (1240) together in a single step may advantageously simplify procedure setup. The user may engage the connector (1220), for example, using the tabs (1222), and may move the connector (1220) axially to further separate the connector (1220) (and the external fluid device (1240) coupled thereto) from the delivery device (1200). In some variations, the connector (1220) and the external fluid device (1240) may be disposed of and the delivery device (1200) may be used to deliver the fluid composition to eye as will be described in more detail below. It should be appreciated that while described above as a single removal step (e.g., removing the connector and the external fluid device together), the two components may be released from the handle of the delivery device separately instead (e.g., the external fluid device may be disengaged from the connector, and the connector may then be disengaged from the housing of the handle).

### Performing Ocular Procedure

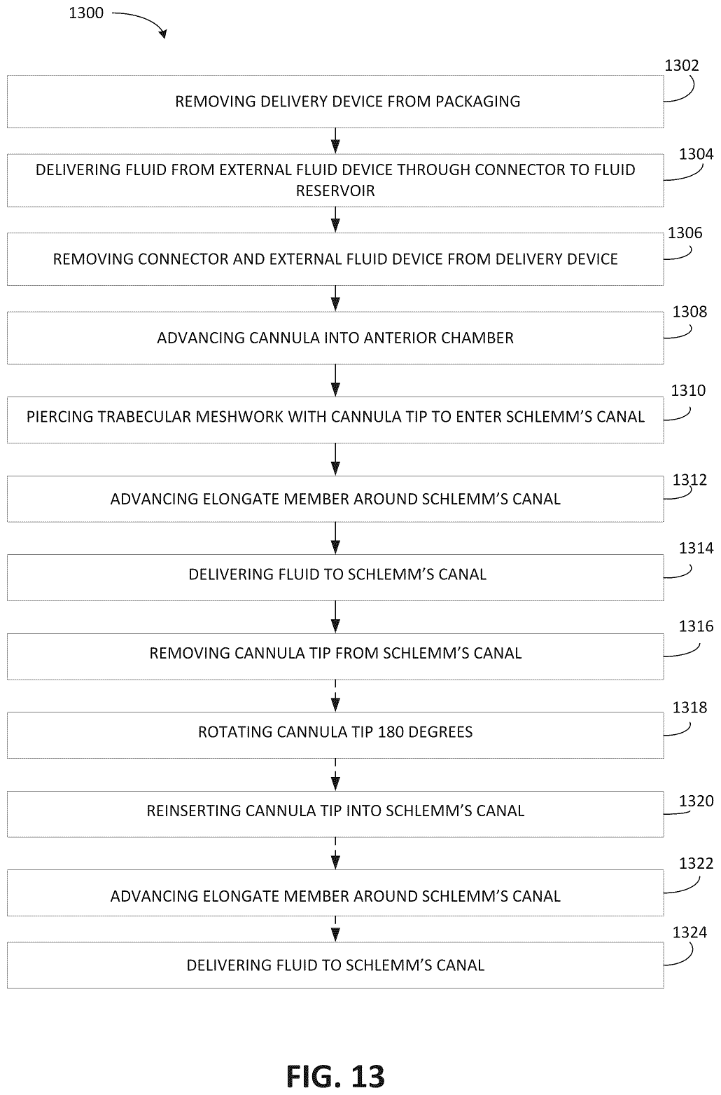

FIG. 13 illustrates a flow chart of an exemplary method (1300) of treating conditions of the eye including delivering fluid to an eye. In some variations, the delivery device of a fluid delivery system may be contained within packaging, such as, a single use packaging. In these variations, methods may include removing the delivery device from the packaging (1302). As described above, the delivery device may or may not be packaged with the connector coupled to the housing of the handle. In variations in which the delivery device is packaged with the connector decoupled, or the connector is otherwise decoupled upon preparation of the delivery device for use, methods may include coupling the connector to the handle at the proximal opening. In some variations, coupling the connector to the housing of the handle includes releasably coupling the connector to the coupling hub within the proximal cavity of the handle. In some variations, coupling the connector to the housing of the handle at the proximal opening includes inserting the connector into the proximal cavity of the handle and rotating or twisting the connector in a first direction.

The method (1300) may further comprise delivering fluid from an external fluid device through the connector to the fluid reservoir (1304). Delivering fluid from the external fluid device through the connector to the fluid reservoir may include delivering a fluid composition from the external fluid device through the connector releasably coupled to the fluid reservoir contained within the housing of the handle of a delivery device, the fluid reservoir in fluid communication with an elongate member slidably positioned within a cannula coupled to the handle. Delivering fluid from the external fluid device through the connector to the fluid reservoir may include coupling the external fluid device to the connector to deliver to the fluid composition from the external device to the fluid reservoir. In some variations, the external fluid device may be detachably or releasably coupled to the connector as described above. For example, in some variations, the external fluid device may be detachably coupled to the connector in a threaded engagement. In some variations, delivering fluid from the external fluid device to the fluid reservoir may include delivering the fluid composition through the connector to the fluid reservoir until the fluid composition is received at the distal end of the cannula or the fluid reservoir is filled, and any remaining air has been purged from the cannula and the elongate member.

In some variations, the method (1300) may further comprise removing the connector and the external fluid device from the delivery device (1306). In some variations, removing the connector and the external fluid device from the delivery device may include removing the connector and the external fluid device while the external fluid device is coupled to the connector. In some variations, removing the connector and the external fluid device from the delivery device may include rotating the connector in the second direction opposite the first direction and/or axially retracting the connector, and removing the connector from a proximal cavity of the handle. Removing the connector and the external fluid device from the delivery device may include rotating the connector (e.g., using the one or more tabs of the connector) in the second direction opposite the first direction. In some variations, removing the connector and the external fluid device from the delivery device may include rotating the connector to release the connector and the external fluid device from the handle.

In some variations, the methods may generally include steps of creating an incision in the ocular wall (e.g., the sclera or cornea) that provides access to the anterior chamber of the eye, advancing a cannula of the delivery system through the incision and at least partially across the anterior chamber to the trabecular meshwork, accessing Schlemm's canal with the cannula. The method as described above may also include the step of priming or flushing the system with the fluid composition (e.g., to remove air from the system) and/or the step of irrigating the operative field to clear away blood or otherwise improve visualization of the field. The surgeon may first view the anterior chamber and trabecular meshwork (with underlying Schlemm's canal) using an operating microscope and a gonioscope or gonioprism. Using a 0.5 mm or greater corneal, limbal, or sclera incision, the surgeon may then gain access to the anterior chamber. A saline solution or viscoelastic composition may then be introduced into the anterior chamber to prevent its collapse. Here the saline solution or viscoelastic composition may be delivered through the cannula of the delivery system or by another mode, e.g., by infusion through an irrigating sleeve on the cannula. The surgeon, under direct microscopic visualization, may then advance the cannula of the delivery system through the incision towards the anterior chamber angle. When nearing the angle (and thus the trabecular meshwork), the surgeon may apply a gonioscope or gonioprism to the cornea to visualize the angle. The application of a viscous fluid (e.g., a viscoelastic composition as previously described) to the cornea and/or gonioscope or gonioprism may help to achieve good optical contact and negate total internal reflection thereby allowing visualization of the anterior chamber angle. As the surgeon visualizes the trabecular meshwork, the cannula may then be advanced so that the distal tip of the cannula pierces the meshwork and is in communication with the lumen of Schlemm's canal.

The method (1300) may further include advancing the cannula into the anterior chamber of the eye (1308) and piercing the trabecular meshwork with the distal tip of the cannula to enter Schlemm's canal (1310). In some variations, piercing the trabecular meshwork with the cannula tip may include piercing the trabecular meshwork with the distal tip including any variation described above, including in FIGS. 11A-11L. In some variations, piercing the trabecular meshwork with the distal tip may include lifting the trabecular meshwork with the distal edge to slide the trabecular meshwork up a portion of the distal tip. Sliding the trabecular meshwork up a portion of the distal tip may create an access point in the trabecular meshwork though which an elongate member may be advanced. Piercing the trabecular meshwork with the distal tip may include piercing the trabecular meshwork with a distal tip having a distal edge including a straight portion, a curved portion having a compound curve with the two or more curves bending in the same direction and a proximal edge. In some variations, piercing the trabecular meshwork with the distal tip may include piercing the trabecular meshwork with the distal tip, where the distal tip comprises a straight portion, a curved portion comprising a reverse curve with the two or more curves where at least one curve bends in a first direction and at least one curve bends in a second direction different from the first direction. In some variations, the distal tip may include a straight portion comprising a first straight portion distal to a second straight portion. In some variations, piercing the trabecular meshwork with the distal tip may include sliding the trabecular meshwork up the straight portion and a portion of the curved portion of the distal tip to provide an access point in the trabecular meshwork through which the elongate member may be advanced.

In some variations, the methods may generally include the slidable elongate member coaxially disposed within the cannula lumen being advanced into the canal under gonioscopic visualization. The method (1300) may further include advancing the elongate member around Schlemm's canal (1312). The elongate member may be advanced any suitable amount and direction about the canal. For example, the elongate member may be advanced between about 1 degree and about 360 degrees about the canal, between about 10 degrees and about 360 degrees about the canal, between about 150 and about 210 degrees about the canal, or any suitable distance, about 360 degrees about the canal, about 270 degrees about the canal, about 180 degrees about the canal, about 120 degrees about the canal, about 90 degrees about the canal, about 60 degrees about the canal, about 30 degrees about the canal, or about 5 degrees about the canal. In some variations, the elongate member may be advanced in two steps, e.g., first in a clockwise direction (e.g., about 180 degrees, about 90 degrees, etc.) and second in a counterclockwise direction (e.g., about 180 degrees, about 90 degrees, etc.) about the canal (e.g., to thereby achieve a 360- or 180-degree ab-interno viscocanalostomy or canaloplasty). In some variations, the elongate member may be advanced in one step (e.g., about 90 degrees in a clockwise direction, about 180 degrees in a clockwise direction, about 270 degrees in a clockwise direction, about 360 degrees in a clockwise direction, about 90 degrees in a counterclockwise direction, about 180 degrees in a counterclockwise direction, about 270 degrees in a counterclockwise direction, about 360 degrees in a counterclockwise direction) about the canal to thereby achieve a corresponding degree ab-interno viscocanalostomy or canaloplasty. Advancing the elongate member around Schlemm's canal includes using the drive assembly to advance the elongate member around Schlemm's canal. In some variations, using the drive assembly to advance the elongate member around Schlemm's canal includes using the one or more actuators including the one or more wheels, the slide, or the button as described herein to advance or retract the elongate member around Schlemm's canal.

The method (1300) further includes delivering fluid (e.g., the fluid composition) to Schlemm's canal (1314). The fluid composition may be injected upon advancement or retraction of the elongate member. Once the slidable elongate member has been positioned within the canal, a fluid composition, e.g., a viscoelastic solution, may be continuously or intermittently delivered through the lumen of the elongate member. The fluid composition may exit the lumen of the elongate member through its distal end (e.g., the through the distal tip), or through openings or fenestrations provided along its shaft, or a combination of both. The openings or fenestrations may be spaced along the axial length of the elongate member in any suitable manner, e.g., symmetrically or asymmetrically along its length. Other substances such as drugs, air, or gas may delivered be in the same manner if desired. In some variations, retracting the elongate member may include using the first actuator to retract the elongate member and deliver fluid to Schlemm's canal simultaneously, while in other variations, the retraction of the elongate member and delivery of fluid may be entirely decoupled. Accordingly, in some variations, the method may include delivering fluid to Schlemm's canal, then retracting the elongate member, or vice versa. This combination of delivering fluid and retracting the elongate member (and/or advancing the elongate member) may be completed any suitable number of times during a procedure. It should be appreciated that the elongate member may be advanced and/or retracted any desirable amount around Schlemm's canal to disrupt the tissues and appropriately place the elongate member for fluid delivery. Any suitable number of actuators may be used to advance the elongate member, retract the elongate member, and deliver fluid, including using a single actuator for all, individual actuators for all, or a combination thereof. For example, in some variations, retracting the elongate member may include using a second separate actuators to retract the elongate member and to deliver fluid to Schlemm's canal.

In some methods, an elongate member comprising a lumen may be advanced into Schlemm's canal and the fluid composition may be delivered via the elongate member. The elongate member and/or fluid delivery may dilate Schlemm's canal, and fluid delivery may additionally dilate the collector channels. The entire length of Schlemm's canal or a portion thereof may be dilated by the fluid. For example, at least 75%, at least 50%, at least 25%, at least 10% of the canal, or at least 1% of the canal may be dilated. The fluid compositions may also be delivered to treat various medical conditions of the eye, including but not limited to, glaucoma, pre-glaucoma, anterior or posterior segment neovascularization diseases, anterior or posterior segment inflammatory diseases, ocular hypertension, uveitis, age-related macular degeneration, diabetic retinopathy, genetic eye disorders, complications of cataract surgery, vascular occlusions, vascular disease, or inflammatory disease.

In some variations, the slidable elongate member may be repositioned by retraction or repeated advancement and retraction. In some variations of the method, the same or different incision may be used, but the delivery system cannula is employed to access and dilate Schlemm's canal from a different direction (e.g., counterclockwise instead of clockwise). Once a sufficient amount of fluid has been delivered, the surgeon may retract the slidable elongate member into the cannula and remove the delivery system from the eye. It should be appreciated that the cannulas described here may be specifically manufactured to comprise a dual-surface configuration at the distal tip (i.e., sharp and smooth surfaces), which may allow the elongate member to be advanced, repositioned, and/or retracted without severing it on the distal tip of the cannula. It should also be understood that these steps may be used alone or in combination with cataract surgery (in one sitting).

More generally, in methods described herein, exemplary volumes of viscoelastic fluid that may be delivered may in some instances be between about 1 μl and about 200 μl, or in some instances be between about 1 μl and about 100 μl. In some instances, sufficient volumes to provide a disruptive force may range from about 1 μl to about 50 μl, from about 1 μl to about 30 μl, from about 2 μl to about 16 μl, from about 5 μl to about 25 μl, or from about 8 μl to about 21 μl. In one variation, a volume of about 4 μl is sufficient to disrupt Schlemm's canal and/or the surrounding tissues. In other variations, the volume of viscoelastic fluid sufficient to disrupt trabeculocanalicular tissues may be about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 35 μl, about 40 μl, about 45 μl, or about 50 μl. In other variations, the volume of viscoelastic fluid sufficient to disrupt trabeculocanalicular tissues may be between about 50 μl and about 100 μl, including, for example, about 55 μl, about 60 μl, about 65 μl, about 70 μl, about 75 μl, about 80 μl, about 85 μl, about 90 μl, about 95 μl or about 100 μl.

The method (1300) may include removing the distal tip of the cannula from Schlemm's canal (1316). Removing the distal tip of the cannula from Schlemm's canal may include removing the distal tip of the cannula from the anterior chamber of the eye and rotating the distal tip. In some variations, wherein fluid has not been delivered to the entirety of Schlemm's canal, the method (1300) may include optional steps of rotating the cannula 180 degrees (1318) and reinserting the distal tip into Schlemm's canal (1320). In some variations, rotating the cannula 180 degrees includes rotating the handle 180 degrees to rotate the cannula 180 degrees. In some variations, rotating the cannula 180 degrees may comprise rotating the cannula itself 180 degrees independently of (e.g., without) rotating the handle.

The method (1300) may further include advancing the elongate member around Schlemm's canal (1322) and delivering fluid to Schlemm's canal (1324). Advancing the elongate member around Schlemm's canal may include advancing the elongate member the rest of the way around Schlemm's canal including for example between about 0 degrees to about 360 degrees around Schlemm's canal. Advancing the elongate member around Schlemm's canal includes using the drive assembly to advance the elongate member around Schlemm's canal. As described above, using the drive assembly to advance the elongate member around Schlemm's canal includes using the one or more mechanical actuators to advance the elongate member around Schlemm's canal including the one or more wheels, the slide, or the button. Retracting the elongate member as described above, delivering fluid to Schlemm's canal may include delivering fluid to Schlemm's canal during advancement or retraction of the elongate member. Advancement or retraction of the elongate member includes using the first mechanical actuator or the second separate mechanical actuator to advance or retract the elongate member and deliver fluid to Schlemm's canal.

Some of the delivery systems described herein may be configured such that the cumulative amount of advancement and/or retraction of the slidable elongate member is limited. For example, as described above, after the elongate member may be advanced and retracted a particular cumulative distance (e.g., about 39 mm to about 40 mm each of advancement and retraction, corresponding to the approximate circumference of Schlemm's canal; or about 78 mm to about 80 mm each of advancement and retraction, corresponding to approximately twice the circumference of Schlemm's canal; or any other suitable distance), the elongate member may no longer be able to be advanced. This advancement and retraction may occur over multiple advancement-retraction cycles. For example, the elongate member may be advanced about 20 mm, then retracted by about 20 mm, then advanced by about 20 mm, then retracted by about 20 mm. When the cumulative distance is limited to about 40 mm, after these two cycles of advancement and retraction, the elongate member may no longer be able to be advanced. In other variations, the delivery systems may not limit the cumulative amount of advancement and/or retraction of the elongate member.

In some variations of the ab-interno method, the fluid composition may be delivered simultaneously with retraction of the elongate member (i.e., the fluid compositions may be delivered in a manner where retraction of a system component allows advancement of the fluid out of the system cannula). It should be understood that the delivery systems may be configured so that the fluid compositions are delivered continuously, passively, automatically, or actively by the surgeon. The fluid compositions may also be delivered to the canal independent of the gear shaft movement with a pump or auxiliary plunger. In some variations, retraction of the elongate member may correspond to a fixed volume of fluid composition being delivered via the lumen of the elongate member. The fluid composition may be delivered via the distal opening of the lumen of the elongate member as it is retracted, and thus, the fluid may be evenly delivered throughout the portion of the canal through which the elongate member was advanced.

The fluid compositions that may be delivered by the systems described herein include but are not limited to saline and viscoelastic fluids. The viscoelastic fluids may comprise hyaluronic acid, chondroitin sulfate, cellulose, derivatives or mixtures thereof, or solutions thereof. In one variation, the viscoelastic fluid comprises sodium hyaluronate. In another variation, the viscoelastic composition may further include a drug. For example, the viscoelastic composition may include a drug suitable for treating glaucoma, reducing or lowering intraocular pressure, reducing inflammation, fibrosis neovascularization or scarring, and/or preventing infection. For example, in some variations, the viscoelastic composition may include the therapeutic agents described herein, such as but not limited to Rho kinase (ROCK) inhibitors and agents for gene therapy, DNA, RNA, or stem cell-based approaches.

The viscoelastic fluids may also include agents that aid with visualization of the viscoelastic fluids. For example, dyes such as but not limited to fluorescein, trypan blue, or indocyanine green may be included. In some variations, a fluorescent compound or bioluminescent compound is included in the viscoelastic composition to help with its visualization. In other variations, the system may deliver the drug alone, without the viscoelastic composition. In this case, the drug may be loaded onto or into a sustained release biodegradable polymer that elutes drug over a period of weeks, months, or years. It is also contemplated that air or a gas could be delivered with the systems, as described herein.

The viscoelastic fluid may be delivered while advancing the elongate member of a single-handed, single-operator controlled device from Schlemm's canal in the clockwise direction, counterclockwise direction, or both, or during withdrawal of the elongate member from Schlemm's canal. As previously stated, the viscoelastic fluid may be delivered to disrupt Schlemm's canal and surrounding trabeculocanalicular tissues. For example, the delivered viscoelastic fluid may cause disruption by dilating Schlemm's canal, increasing the porosity of the trabecular meshwork, stretching the trabecular meshwork, forming microtears or perforations in juxtacanalicular tissue, removing septae from Schlemm's canal, dilating collector channels, or a combination thereof. The elongate member may be loaded with the viscoelastic fluid at the start of an ocular procedure so that the fluid can be delivered by a single device. This is in contrast to other systems that use forceps or other advancement tool to advance a fluid delivery catheter into Schlemm's canal and/or devices containing viscoelastic fluid that are separate or independent from a delivery catheter or catheter advancement tool, and which require connection to the delivery catheter or catheter advancement tool during a procedure by an assistant while the delivery catheter or catheter advancement tool is held by the surgeon.

Some of the methods, described in more herein, may comprise dilating Schlemm's canal and/or aqueous collector channels (e.g., with viscoelastic fluid) using the delivery systems described herein. The methods may also comprise tearing or cutting the trabecular meshwork of Schlemm's canal. These methods may be carried out separately, or they may be combined into a single procedure. For example, in some instances a portion (e.g., half) of Schlemm's canal may be dilated (using a fluid composition, for example), and the trabecular meshwork of the same or a different portion of Schlemm's canal may be torn or cut, within the same eye. As another example, all of Schlemm's canal may be dilated, and then all or a portion of the trabecular meshwork may subsequently be torn or cut. This may be desirable, for example, in order to both dilate the collector channels and tear or cut the trabecular meshwork.

In some of these variations, dilation and tearing or cutting may be performed using a single delivery system, such as one described herein configured to deliver a fluid composition. For example, the elongate member of a delivery system configured to deliver a fluid composition may first be used to deliver a fluid composition to a portion of Schlemm's canal (e.g., about a 180-degree arc of the canal, about a 90-degree arc of the canal) as described herein, and subsequently to tear or cut the trabecular meshwork in the same portion of the canal as described herein. As another example, the conduit of a delivery system configured to deliver a fluid composition may first be used to deliver a fluid composition to a portion of Schlemm's canal (e.g., about a 180-degree arc of the canal, about a 90-degree arc of the canal, etc.) and subsequently to tear or cut the trabecular meshwork in another portion of the canal (e.g., the other about-180 degree arc, another 90 degree arc, etc.). As yet another example, the elongate member of a delivery system configured to deliver a fluid composition may first be used to deliver fluid composition to all of Schlemm's canal (e.g., by delivering about 180 degrees of fluid composition in a first direction and then delivering about 180 degrees of fluid composition in a second direction), and then subsequently to tear or cut the full 360 degrees of trabecular meshwork (e.g., by tearing or cutting about 180 degrees of trabecular meshwork in a first direction and then tearing or cutting about 180 degrees of trabecular meshwork in a second direction). In another example, the elongate member of a delivery system configured to deliver a fluid composition may first be used to deliver fluid composition to all of Schlemm's canal in one step (i.e., by delivering about 360 degrees of fluid composition to Schlemm's canal in a single direction), and then subsequently to tear or cut the full 360 degrees of trabecular meshwork in a single step (i.e., by tearing or cutting about 360 degrees of trabecular meshwork in a single direction).

In other variations, dilation and tearing or cutting may be performed using different delivery systems (e.g., the dilation may be performed using a delivery system configured to deliver a fluid composition, and the tearing or cutting may be performed using a delivery system not configured to deliver a fluid). In another example, in some instances, dilation may be performed in one eye of a patient, while the trabecular meshwork may be torn or cut in the other eye of the patient.

Procedures comprising dilating Schlemm's canal and/or tearing or cutting the trabecular meshwork may also be combined with procedures delivering an ocular device, either in the same eye or in a different eye of the same patient. For example, all or a portion of Schlemm's canal may be dilated, followed by insertion of an ocular device. As another example, a portion of the trabecular meshwork may be torn or cut, while an ocular implant may be delivered to another portion of Schlemm's canal. In another example, a portion of Schlemm's canal may be dilated, while an ocular implant may be delivered to another portion of Schlemm's canal. In another example, an ocular implant may be delivered to a portion of Schlemm's canal, and then Schlemm's canal may be subsequently dilated to improve the function of the ocular implant.

Any suitable ocular device that maintains the patency of Schlemm's canal or improves outflow of aqueous humor may be implanted. For example, ocular devices that maintain the patency of Schlemm's canal without substantially interfering with fluid flow across, along, or out of the canal may be implanted. Such devices may comprise a support having at least one fenestration, as disclosed in U.S. Pat. Nos. 7,909,789, and 8,529,622, which were previously incorporated by reference in their entirety. Ocular devices that disrupt the juxtacanalicular trabecular meshwork or adjacent inner wall of Schlemm's canal may also be implanted. In addition to ocular devices made from metal or metal alloys, the use of sutures, modified sutures, modified polymers, polymeric filaments, or solid viscoelastic structures may be delivered.

Other variations of the ab-interno method include the use of an endoscope. Similar to the method described directly above, access to the anterior chamber is first made by incising the cornea, limbus, or sclera. Again, this may be done in combination with cataract surgery in one sitting, either before or after cataract surgery, or independently. The anterior chamber may be infused with saline solution or a viscoelastic composition may be placed in the anterior chamber to prevent its collapse. The saline or viscoelastic may be delivered as a separate step or it may be infused with the elongate member of the delivery system, an irrigating sleeve on the elongate member or cannula, or with a separate infusion cannula. The surgeon, under direct microscopic visualization, then advances the endoscope through the incision and towards the angle and trabecular meshwork. As the surgeon visualizes the trabecular meshwork via the endoscope or any associated display, the bevel of the cannula is advanced to pierce the meshwork. The elongate member is then advanced under endoscopic visualization. The elongate member may be advanced any suitable amount and direction about the canal. For example, the elongate member may be advanced between about 10 degrees to about 360 degrees about the canal, or it may be advanced in two steps, e.g., 180 degrees in a clockwise direction and 180 degrees in a counterclockwise direction about the canal (to thereby achieve a full 360 degree ab-interno viscocanalostomy). Once the elongate member has been positioned within the canal, a fluid composition, e.g., a viscoelastic fluid, may be continuously or intermittently delivered through the lumen of the elongate member. The fluid composition may exit the lumen of the elongate member through its distal end (e.g., the through the distal tip), or through openings or fenestrations provided along its shaft, or a combination of both. The openings or fenestrations may be spaced along the axial length of the elongate member in any suitable manner, e.g., symmetrically or asymmetrically along its length. Other substances such as drugs, air, or gas may be delivered in the same manner if desired. The elongate member may be repositioned by retraction or repeated advancement and retraction. In some variations of the method, the same or different incision may be used, but the delivery system cannula is employed to access and dilate Schlemm's canal from a different direction (e.g., counterclockwise instead of clockwise). Once a sufficient amount of fluid has been delivered, the surgeon may retract the slidable elongate member into the cannula. In some variations the surgeon may then remove the delivery system from the eye; in other variations the surgeon may keep the delivery system within the eye and perform a trabeculotomy, as described in more detail herein.

Tissue disruption may occur by viscodilating excessively and intentionally with at least about 1 μl, at least about 2 μl, at least about 3 μl, at least about 4 μl, at least about 5 μl, at least about 6 μl, at least about 7 μl, at least about 8 μl, at least about 9 μl, at least about 10 μl, at least about 11 μl, at least about 12 μl, at least about 13 μl, at least about 14 μl, at least about 15 μl, at least about 16 μl, at least about 17 μl, at least about 18 μl, at least about 19 μl, at least about 20 μl, at least about 21 μl, at least about 22 μl, at least about 23 μl, at least about 24 μl, at least about 25 μl, at least about 26 μl, at least about 27 μl, at least about 28 μl, at least about 29 μl, at least about 30 μl, at least about 35 μl, at least about 40 μl, at least about 45 μl, or at least about 50 μl viscoelastic fluid per 360 degree arc of the canal. In some variations, at least about 20 μl, at least about 25 μl, at least about 30 μl, at least about 35 μl, at least about 40 μl, at least about 45 μl, or at least about 50 μl of viscoelastic fluid may be delivered. In other variations, at least about 55 μl, about 60 μl, about 65 μl, about 70 μl, about 75 μl, about 80 μl, about 85 μl, about 90 μl, or about 100 μl viscoelastic fluid may be delivered.

Depending on factors such as the type or severity of the condition being treated, the disruptive force may be generated to partially or completely destroy and/or remove the trabecular meshwork and may be adjusted by varying the volume of viscoelastic fluid delivered.

Additionally, the fluid compositions may be delivered to restore the tubular anatomy of Schlemm's canal, to clear obstructions within the canal, to disrupt juxtacanalicular trabecular meshwork or the inner wall of Schlemm's canal within the canal, or to expand the canal. Here the delivery systems may include wires, tubes, balloons, instruments that deliver energy to the tissues, and/or other features to help with these methods. It is contemplated that glaucoma may be treated using such systems with additional features. The surface of these systems may also be roughened or have projections to further disrupt the inner wall of Schlemm's canal and juxtacanalicular trabecular meshwork to enhance aqueous humor outflow or permeability. Additionally, it should be appreciated that the delivery systems described herein may be used to deliver the fluid compositions to the anterior chamber or anterior segment.

### Trabeculotomy

The method described herein may comprise performing a trabeculotomy. The methods (as well as systems and devices) described herein, including the method for providing a disruptive force to trabeculocanalicular tissues, may be highly suitable for ab-interno trabeculotomy and goniotomy given that they avoid the use of electrocautery, and are capable of advancing elongate members over larger degrees of arc of Schlemm's canal. In some instances, disruptive tools may comprise disruptive components on their distal portions. Exemplary disruptive components include, without limitation, notches, hooks, barbs, balloons, or combinations thereof. In other instances, the disruptive tools may not comprise disruptive components on their distal portions, and indeed may have atraumatic blunt distal portions. Exemplary atraumatic distal portions include, without limitation, parasol or dome shaped distal portions.

The outer diameter of the elongate member or tool may be variously sized for disruption of tissues, analogous to how fluid volumes may be varied to vary the level of disruption. For example, an elongate member or tool having an outer diameter ranging from about 50 to about 100 microns may be advanced through the canal to slightly dilate the canal and break or remove septae obstructing circumferential canalicular flow. An elongate member or tool having an outer diameter ranging from about 100 to 200 microns may be employed to perform the foregoing and may also to begin to stretch the trabecular meshwork and juxtacanalicular tissues. An elongate member or tool having an outer diameter ranging from about 200 to about 300 microns may be able to perform the above but may also create microtears in the trabecular meshwork and juxtacanalicular tissues and may maximally dilate the collector channels. An elongate member or tool having an outer diameter ranging from about 300 to about 500 microns may maximally disrupt the tissues and may create tears or perforations all along the trabecular meshwork and juxtacanalicular tissues. The elongate member or tool may be advanced out from the tip of the cannula and into the canal about a 30 degree arc of the canal (e.g., advanced about 3 to 4 mm out of the cannula), advanced about a 60 degree arc of the canal (e.g., advanced about 6 to 8 mm out of the cannula), advanced about a 90 degree arc of the canal (e.g., advanced about 10 mm out of the cannula), advanced about a 120 arc of the canal (e.g., advanced about 15 mm out of the cannula), advanced about a 180 degree arc of the canal (e.g., advanced about 20 mm out of the cannula), or advanced about a full 360 degrees of the canal (e.g., advanced about 36 to 40 mm out of the cannula), for maximal intraocular pressure reduction. In some variations, the elongate member may have a non-uniform outer diameter. For example, the elongate member may have a tapered outer diameter, such that the outer diameter increases from the distal to proximal end.

In some variations, the methods disclosed herein may include advancement of the elongate member between about a 5-degree arc of Schlemm's canal and about a 360-degree arc. In some variations, the methods may include advancement of the elongate member (or tool) about a 360-degree arc of Schlemm's canal, about a 270-degree arc of Schlemm's canal, about a 120-degree arc of Schlemm's canal, about a 180-degree arc of Schlemm's canal, or about a 90-degree arc of Schlemm's canal. In yet further variations, advancement of the elongate member (or a tool) may be about a 0-to-5-degree arc of Schlemm's canal, about a 30-degree arc of Schlemm's canal, or about a 60-degree arc of Schlemm's canal. Advancement may occur from a single access point in Schlemm's canal or from multiple access points in the canal. It may be beneficial to advance the elongate member in both clockwise and counterclockwise directions about a 180-degree arc of Schlemm's canal from a single access point in the canal. In other variations, the elongate member may be advanced in a single (clockwise or counterclockwise) direction about 360 degrees of Schlemm's canal from a single access point in the canal.

Depending on factors such as the type or severity of the condition being treated, the disruptive force may be generated to partially or completely destroy and/or remove the trabecular meshwork and may be adjusted by varying the tool configuration. In some methods, the trabecular meshwork may be disrupted during advancement of the slidable elongate member. Customizing a body segment of the elongate member proximal to the tip with one or more notches, barbs, or balloons that catch the meshwork as the distal tip is being guided and advanced along Schlemm's canal may also be used, thereby disrupting, partially tearing, fully tearing, and/or removing trabecular meshwork upon advancement. Additionally, an implant with edges specifically designed to cut the meshwork may be used.

In yet other methods, the trabecular meshwork may be disrupted during retraction of the slidable elongate member. The methods for disrupting tissues may involve customizing the system (e.g., the elongate member, any catheters or wires, probe tips, etc.) to catch or grasp the meshwork upon retraction after advancement through the canal. This may be done using a wire with a bent tip, hook, notch, or barb on its end that is advanced through the lumen of the catheter that then snags the meshwork upon retraction, tearing it along its length or removing it altogether, or solely with a metal or polymer wire or suture (no catheter) whose tip (and/or body) is hooked, notched, or barbed in such a way that it can be advanced into Schlemm's canal without tearing the meshwork but snags the meshwork upon retraction, tearing the meshwork and/or removing it completely. The elongate member may be provided with a disruptive tool, e.g., a sharp-edged element, that can cut or tear the trabecular meshwork while being retracted into the cannula, which is held stationary. Exemplary sharp-edged elements may be a hook, wire, or any other suitable shape memory component that can extend from the cannula to tear, cut, or remove trabecular meshwork.

Another method for disrupting tissues may include using oversized elongate members (e.g., having an outside diameter of 300-500 microns) to tear the meshwork upon delivery, or inflating or expanding the elongate member once it has been fully advanced into Schlemm's canal to stretch, disrupt, rupture, or fully tear the meshwork. For example, a catheter/elongate member, probe, or wire (with or without a lumen) whose tip is 200-250 microns in outer diameter, but having a shaft that begins to flare outwards after 3 clock hours of Schlemm's canal (i.e., at about the 5 or 10 mm mark on the catheter/elongate member) up to about 300, up to about 400, or up to about 500 microns, may be used, so that as the tip advances comfortably within Schlemm's canal, the enlarged shaft trails behind and ruptures the trabecular meshwork as it is advanced.

In another method, cutting, destruction, removal, or the like of the trabecular meshwork may be accomplished by removing the cannula from the eye while leaving the elongate member in the canal, thereby tearing through the meshwork. For example, a cannula may be inserted into the anterior chamber and Schlemm's canal, and a tool (e.g., a slidable elongate member) may be advanced within the canal. The cannula may be removed from the anterior chamber without retracting the elongate member. This action by itself may tear the trabecular meshwork. As the cannula is removed from the anterior chamber, the elongate member may begin tearing the trabecular meshwork from the point at which the cannula was inserted into Schlemm's canal and may continue tearing around the trabecular meshwork toward the distal end of the elongate member.

The methods described here may be used to access the trabecular outflow system using a single clear corneal incision and may allow for transluminal trabeculotomy of up to 360 degrees. The method may use a flexible elongate member that may be advanced and retracted using a single-handed disposable manual instrument. In one variation of the method, the cannula may be held securely against the angle while the flexible elongate member is advanced into Schlemm's canal. An exposed portion of one or more of the wheels may be rotated proximally to advance the flexible elongate member up to about 180 degrees around Schlemm's canal (about 20 mm of circumferential canal travel). For example, the elongate member may be advance about 90, 135, or 180 degrees. At this point, the flexible elongate member may in some instances be fully extended, and the wheel may no longer be able to be rotated. During this procedure, direct microscopic or gonioscopic visualization of the cannula tip may be maintained, and the anterior chamber may be maintained with viscoelastic or continuous balanced salt solution infusion. Once the flexible elongate member is advanced, the cannula may be removed from the eye through the incision without retracting the flexible elongate member. This may cause the body of the flexible elongate member to tear or cut through the trabecular meshwork. In some instances, it may be desirable to bias the distal tip of the cannula toward the trabecular meshwork being cut; this may in some instances help to prevent the flexible elongate member from slipping out of the canal during cannula removal.

The methods are generally single-handed, single-operator controlled methods that are minimally invasive, e.g., they are tailored for an ab-interno procedure, which as previously mentioned, can be advantageous over the more invasive ab-externo approach. However, use of the systems in an ab-externo method may be contemplated in some instances and thus, are not excluded here. The methods may be used to treat or prevent glaucoma, pre-glaucoma, or ocular hypertension. When treating glaucoma, the methods may also be used in conjunction with a cataract surgery (before or after) using the same incision during the same session or at another time.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While variations of the present invention have been shown and described herein, those skilled in the art will understand that such variations are provided by way of example only. Numerous variations, changes, and substitutions will now occur to those skilled in the art without departing from the invention. It should be understood that various alternatives to the variations of the invention described herein may be employed in practicing the invention. It is intended that the following claims define the scope of the invention and that methods and structures within the scope of these claims and their equivalents be covered thereby.

1.-68. (canceled)

69. A device for treating conditions of the eye, comprising:
a handle, the handle comprising a housing having a proximal portion, and a distal portion comprising a grip portion, the grip portion comprising a first curved side, a second curved side opposite the first curved side, and a tapered region distal to the first and second curved sides, wherein the tapered region is configured to receive fingers of the user;
a cannula coupled to a distal end of the handle; and
an elongate member slidably positioned in the cannula and configured to deliver one or more of a fluid and an implant to Schlemm's canal.

70. The device of claim 69, wherein the first curved side and the second curved side are convex.

71. The device of claim 70, wherein the first curved side and second curved side are symmetric across an XZ plane.

72. The device of claim 69, wherein the grip portion comprises an actuator configured to move the elongate member.

73. The device of claim 72, wherein the grip portion further comprises a planar surface proximal of the actuator, the planar surface continuous with an actuator border around the actuator.

74. The device of claim 73, wherein the planar surface is a first planar surface, and the grip portion further comprises a second planar surface, wherein the first planar surface is on a top of the handle and the second planar surface on a bottom of the handle.

75. The device of claim 74, further comprising a second actuator, wherein the first actuator is on the top of the handle, the second actuator is on the bottom of the handle and the second planar surface is proximal of the second actuator.

76. The device of claim 72, wherein the grip portion further comprises a neck proximal of the actuator.

77. The device of claim 76, wherein the proximal portion has a first height at a distal end thereof, the neck has a second height, and the distal portion has a maximum height aligned with at least a portion of the actuator, and wherein the first height and the maximum height are greater than the second height.

78. The device of claim 77, wherein the handle comprises a continuous curve between the maximum height and the second height.

79. The device of claim 69, wherein the handle comprises a fluid reservoir at least partially contained therein.

80. The device of claim 79, wherein the handle comprises a connector releasably coupled to the fluid reservoir in the handle, wherein the connector is configured to receive an external fluid device to transfer fluid into the fluid reservoir, and to be released from the handle with the external fluid device coupled thereto.

81. The device of claim 80, wherein the handle includes a proximal portion comprising a proximal cavity having a proximal opening, the proximal cavity having a coupling hub therein, the coupling hub including a coupling portion.

82. The device of claim 81, wherein the connector engages the coupling portion of the coupling hub.

83. The device of claim 81, wherein the coupling portion includes threads configured to engage a distal luminal wall of the connector.

84. The device of claim 80, wherein the connector comprises a connector body including one or more extensions configured to engage one or more abutments within the proximal cavity of the handle when the connector is coupled to the coupling hub.

85. The device of claim 69, wherein the grip portion includes a textured surface having raised elements, indented elements, or a combination thereof.

86. The device of claim 69, wherein the grip portion comprises two or more cross-sectional shapes along a longitudinal axis of the handle.

87. The device of claim 86, wherein a center section of the grip portion comprises an oval cross-sectional shape with a major axis and a minor axis, the center section of the grip portion including the top surface, the bottom surface, and the one or more actuators.

88. The device of claim 87, wherein the grip portion comprises a circular cross-sectional shape with a first diameter proximal of the center section and a circular cross-sectional shape with a second diameter distal of the center section, wherein the first diameter is greater than the second diameter.

89. The device of claim 69, wherein the cannula comprises a curved proximal portion a curved distal portion, and a distal tip, wherein the curved proximal portion has a first radius of curvature, and the curved distal portion has a second radius of curvature, wherein the first radius of curvature is greater than the second radius of curvature, and wherein the first and second radii of curvature are in opposing directions.

90. The device of claim 88, wherein the distal tip further comprises a distal edge having a straight portion, a proximal edge having a curved portion, and a lumen opening.

91. The device of claim 89, wherein the straight portion of the distal edge and the outer radius form an angle between about 14 degrees to about 20 degrees.

92. The device of claim 89, wherein:
the distal edge comprises an outer distal edge and an inner distal edge defining a tongue, wherein the proximal edge further comprises an inner proximal edge and an outer proximal edge defining a base and wherein the straight portion further comprises a plurality of straight segments, wherein each straight segment comprises a different slope, and
the curved portion of the proximal edge further comprises a plurality of curved segments, wherein each curved segment comprises a different radius of curvature.

93. The device of claim 89, wherein the distal tip of the cannula is aligned with a central longitudinal axis of the handle.

94. The device of claim 93, wherein the distal tip of the cannula is bisected by the central longitudinal axis of the handle.

95. The device of claim 89, wherein the curved proximal portion and the curved distal portion are offset from the central longitudinal axis of the handle.

96. The device of claim 71, wherein the first curved side and second curved side are configured to facilitate user rotation of the handle around a longitudinal axis of the handle.

97. The device of claim 96, wherein the first curved side and the second curved side are configured to facilitate rotation of the handle about 10 degrees to about 180 degrees around the longitudinal axis of the handle.
